(19) United States
(12) Patent Application Publication
(10) Pub. No.: US 2025/0345453 A1
(43) Pub. Date: Nov. 13, 2025

(54) CHEMOTHERAPEUTIC MICELLULAR NANOPARTICLES

(71) Applicant: Peptinovo Biopharma Inc., Ann Arbor, MI (US)

(72) Inventor: Reynold Homan, Ann Arbor, MI (US)

(21) Appl. No.: 19/204,092

(22) Filed: May 9, 2025

Related U.S. Application Data
(60) Provisional application No. 63/645,127, filed on May 9, 2024.

(51) Int. Cl. A61K 47/69; A61K 9/08; A61K 9/19; A61K 47/26; A61K 47/54; A61K 47/64; A61P 35/00; B82Y 5/00

(52) U.S. Cl. CPC A61K 47/6909; A61K 9/08; A61K 9/19; A61K 47/26; A61K 47/544; A61K 47/64; A61P 35/00; B82Y 5/00

(57) ABSTRACT

Disclosed are compounds and compositions that preferentially target cancer cells with a warhead that comprises a chemotherapeutic agent releasably bound to a targeting agent where the chemotherapeutic agent is released upon cellular absorption. Also disclosed are methods of use.

### CROSS-REFERENCE TO RELATED APPLICATIONS

This application claims priority to U.S. Provisional Patent Application Ser. No. 63/645,127 filed May 9, 2024 which application is incorporated herein in its entirety.

### SEQUENCE LISTING

This application incorporates by reference in its entirety the Sequence Listing XML entitled “sequencelisting.xml” (51 kb), which was created on May 9, 2024 and is filed electronically herewith.

### FIELD

This disclosure provides for chemotherapeutic micellular HDL mimetic nanoparticles (“C-m-HDLs”) and compositions comprising such nanoparticles. These C-m-HDLs preferentially target cancer cells as opposed to normal cells. Also disclosed are methods of use, methods for synthesis, and kits of parts comprising such compounds and compositions.

### STATE OF THE ART

Chemotherapeutic drugs by their nature are toxic to cells and have limited target specificity. This means that, upon administration, a portion of such a drug is delivered off-target to healthy tissues or organs causing toxicity thereto. Such off-target delivery also limits the amount of drug administered to a level that such off-target delivery and corresponding damage to healthy tissues and organs is minimized. In addition, some chemotherapeutic agents are fairly water insoluble which limits the amount of drug that can be delivered per unit of time; these include, by way of example only, chemotherapeutic agents such as docetaxel, cisplatin, methotrexate, etoposide, bleomycin, among others. While formulation and other processes can offset some of the poor water solubility of these drugs, providing target specificity to chemotherapeutic agents in such formulations has proven to be elusive. Moreover, issues such as to drug stability, acceptable pK properties, sufficient Cmax and the like must be overcome in order to achieve therapeutic results.

Target specificity is a challenge as the underlying principle of many, if not all, chemotherapeutic drugs is to target cancer cells that rapidly divide-thereby minimizing damage and death to more slowly dividing normal cells. However, cancer cells are not the only rapidly dividing cells: epithelial cells in the stomach and the intestine as well as immune cells also rapidly divide. As such, these non-cancerous cells are suspectable to targeting by chemotherapeutic drugs. Indeed, gastrointestinal complications associated with the off-target (non-specific) effect of chemotherapeutic drugs have limited their use in formulations intended for oral delivery.

Addressing specificity, conjugates of chemotherapeutic agents bound to ligand mimics can be used to enhance tumor target specificity when the receptor being targeted by the ligand mimic is overexpressed by the tumor. Such is the case with SR-BI (Scavenger Receptor class B type I). Mimetics of HDL containing a chemotherapeutic agent can interact with SR-Bi. See, e.g., U.S. Pat. No. 10,532,105, the disclosure of which is incorporated herein by reference in its entirety. While the '105 Patent discloses that compositions described therein are effective in treating cancer, the nanoparticles used therein have an average diameter of about 17 nanometers measured using Dynamic Light Scattering (DLS) techniques.

As to SR-BI, it is a multiligand cellular membrane receptor protein which, in vivo, is the receptor for high density lipoprotein (HDL) and allows for uptake of absorbable lipids (including cholesterol) into cells. Kontush states that HDL is classified into three groups comprising large-average diameter of about 12 nanometers; medium-average diameter of about 9 nanometers; and small-average diameter of about 7.5 nanometers. Kontush, Front. Pharmacol., 6:218 (2015). In turn, Trigatti, et al. states that the binding specificity of HDL to SR-BI is tiered such that larger, less dense HDL particles bind with greater affinity to this receptor than smaller, more dense HDL particles. Trigatti, et al., Atherosclerosis, Thrombosis, and Vascular Biology, 23:1732-1738 (2003). The fact that larger HDL particles are more efficiently bound to SR-BI correlates well with the statements found in Liadaki, et al., J. Biol. Chem., 278 (28): 21262-21271 (2000) regarding particle size and binding affinity to SR-BI. Each of Kontush, Triagatti, et al., and Liadaki, et al. is incorporated herein by reference in its entirety.

Taken together, these references suggest that the ligand mimetics of SR-BI in the '105 Patent are too large and should be reduced, preferably to approximately 12 nanometers in diameter in order to achieve very high affinity to that receptor. In turn, the higher the affinity of a ligand mimic to a receptor, the better the directional guidance to that receptor and, hence, less off-target binding.

Accordingly, there is a continuing need to provide for methods and compositions that allow for the targeted delivery of chemotherapeutic agents for the treatment of cancer. In particular, compositions having a population of C-m-HDLs that have an average diameter that is approximately the size of SR-BI would provide for a higher affinity and, hence, more effective delivery of the chemotherapeutic agent to the cancer cell.

### SUMMARY

In some embodiments, this disclosure describes intermediates, compounds, compositions, manufacturing methods and kits in the treatment of cancer. Such compounds and compositions comprise C-m-HDLs having an average particle size that corresponds substantially to the size of HDL particles that exhibit most efficient uptake by the active site in SR-BI.

In some embodiments, this disclosure describes C-m-HDLs for use in the treatment of solid mass tumors, in a subject in need thereof.

In some embodiments, this disclosure describes C-m-HDLs for use in the treatment of blood borne cancers, in a subject in need thereof.

In some embodiments, this disclosure describes synthetic methods for obtaining a population of C-m-HDLs comprising an average diameter, which substantially corresponds to the size of HDL particles that exhibit most efficient uptake by SR-BI found in vivo.

In some embodiments, there is provided an aqueous composition comprising a population of C-m-HDLs which composition comprises:
a) water;
b) a disaccharide;
c) a population of C-m-HDLs wherein said C-m-HDLs in said population comprise:
   i) an amphiphilic, alpha-helical peptide or protein wherein said peptide or protein forms an HDL mimetic structure on said C-m-HDL;
   ii) one or both of sphingomyelin and phosphatidyl choline, and optionally additional lipid(s) or phospholipid(s); and
   iii) a conjugate comprising an anchor moiety molecule and a chemotherapeutic agent that is releasably attached to each other through a cleavable bond;
wherein the C-m-HDLs in the population comprise a hydrophilic exterior surface and a hydrophobic core; and
further wherein the C-m-HDLs in the population have an average particle diameter of from about 11.5 nanometers to about 14 nanometers as measured by dynamic light scattering.

In some embodiments, there is provided an aqueous composition comprising a population of C-m-HDLs which composition comprises:
a) water;
b) a disaccharide;
c) a population of C-m-HDLs wherein said C-m-HDLs in said population comprise:
   i) an amphiphilic, alpha-helical peptide that comprises an amino acid sequence of any one of SEQ ID NO: 1 through SEQ ID NO:36;
   ii) one or both of sphingomyelin and phosphatidyl choline, and optionally additional lipid(s) or phospholipid(s); and
   iii) a conjugate comprising an anchor moiety molecule and a chemotherapeutic agent that is releasably attached to each other through a cleavable bond;
wherein the C-m-HDLs in the population comprise a hydrophilic exterior surface and a hydrophobic core; and
further wherein the C-m-HDLs in the population have an average particle diameter of from about 11.5 nanometers to about 14 nanometers as measured by dynamic light scattering.

In some embodiments, the population of C-m-HDLs in the above compositions have a mean number average particle diameter of from about 12 nanometers to about 13.5 nanometers.

In some embodiments, at least about 70% of said C-m-HDLs are within plus/minus about 3 nanometers of the mean number average particle diameter of about 12 to about 13.5 nanometers (i.e., from about 9 to about 16.5 nanometers).

In some embodiments, this disclosure describes a population of C-m-HDLs comprising:
(a) an amphiphilic, alpha-helical peptide that comprises an amino acid sequence of any one of SEQ ID NO:25, SEQ ID NO:28, SEQ ID NO:34, SEQ ID NO:35, SEQ ID NO. 36, or combinations thereof;
(b) one or both of sphingomyelin and phosphatidyl choline, and optionally additional lipid(s) or phospholipid(s);
(c) a conjugate comprising an anchor moiety molecule and a chemotherapeutic agent that are releasably attached to each other through a cleavable bond; and
wherein the C-m-HDLs in the population have a hydrophilic exterior surface and a hydrophobic core; and
further wherein the C-m-HDLs in the population have an average particle diameter of from about 11.5 to about 14.0 nanometers, as measured by dynamic light scattering.

In some embodiments, at least about 70% of said C-m-HDLs in said population are within plus/minus about 3 nanometers of the average particle diameter.

In some embodiments, the disaccharide is selected from sucrose, lactose, maltose, trehalose, cellobiose and lactulose.

In some embodiments, the disaccharide comprises any two naturally occurring sugars in an alpha acetal oxygen linkage formation.

In some embodiments, the releasable bond is selected from an ester, a thioester, a carbonate, a thiocarbonate, a carbamate, or a thiocarbamate bond.

In some embodiments, the releasable bond is a carbonate bond.

In some embodiments, cleavage of the carbonate bond removes all vestiges of the linker leaving only chemotherapeutic agent and the anchor moiety. It is contemplated that the carbon oxygen residue is eliminated as carbon dioxide.

In some embodiments, there is provided a population of C-m-HDLs which population comprises:
a) an amphiphilic, alpha-helical peptide that comprises an amino acid sequence of any one of SEQ ID NO: 25, SEQ ID NO:28, SEQ ID NO:34, SEQ ID NO: 35, SEQ ID NO:36, or combinations thereof;
b) one or both of sphingomyelin and phosphatidyl choline, and optionally one or more additional lipids or phospholipids; and
c) a conjugate comprising paclitaxel that is releasably attached to an anchor moiety through a cleavable covalent bond; and
wherein the C-m-HDLs in the population have a hydrophilic exterior surface and a hydrophobic core; and
further wherein the C-m-HDLs have an average particle diameter of from about 12 to about 13.5 nanometers, as measured by dynamic light scattering.

In some embodiments, at least about 70% of said C-m-HDLs in said population are within plus/minus about 3 nanometers or about 2 nanometers of the average particle diameter.

In some embodiments, the disaccharide is selected from sucrose, lactose, maltose, trehalose, cellobiose and lactulose.

In some embodiments, there is provided a population of lyophilized C-m-HDLs as described above, wherein said disaccharide is sucrose.

In some embodiments, the anchor moiety is cholesterol, tocopherol or a tocotrienol the latter two of which include only the β-, γ-, and δ-variants.

In some embodiments, the conjugate includes one of those set forth in Tables 1-6 as follows:

TABLE 1

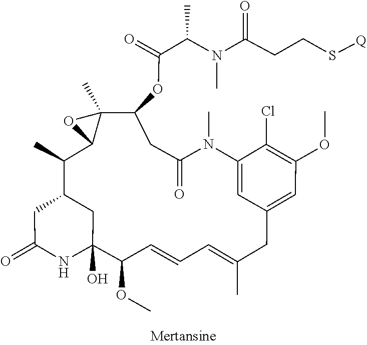

| No. | Q | Trivial Name |
|---|---|---|
| 1 | 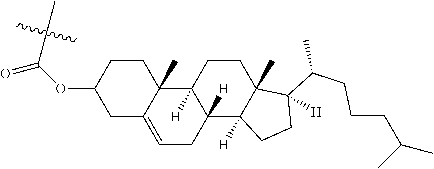 | Mertansine-cholesterol conjugate with a cleavable carbonate bond |
| 2 | 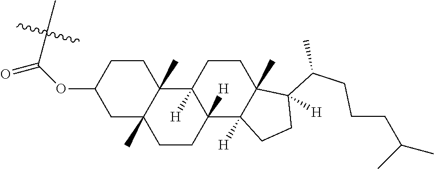 | Mertansine-coprostanol conjugate with a cleavable carbonate bond |
| 3 | 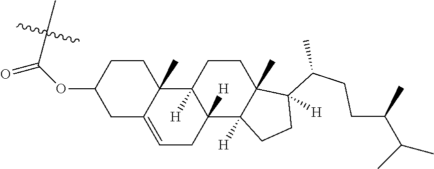 | Mertansine-campesterol conjugate with a cleavable carbonate bond |
| 4 | 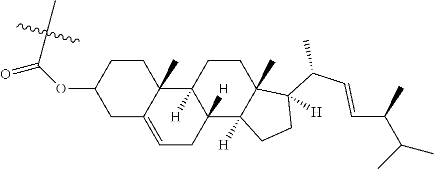 | Mertansine-brassicasterol conjugate with a cleavable carbonate bond |
| 5 | 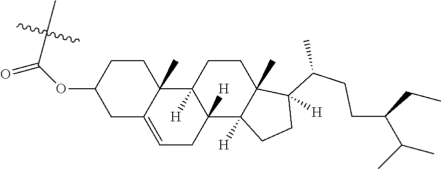 | Mertansine-sitosterol conjugate with a cleavable carbonate bond |
| 6 | 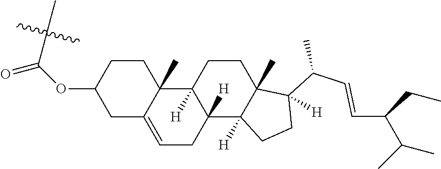 | Mertansine-stigmasterol conjugate with a cleavable carbonate bond |
| 7 | 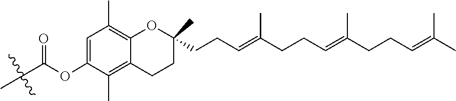 [SMILES: *C(=O)Oc1cc(C)c2c(c1C)CC[C@@](C)(CC/C=C(\C)CC/C=C(\C)CCC=C(C)C)O2] | Mertansine-β-tocotrienol conjugate with a cleavable carbonate bond |
| 8 | 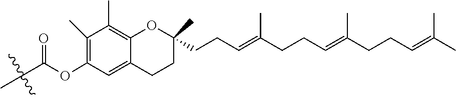 [SMILES: *C(=O)Oc1cc2c(c(C)c1C)O[C@](C)(CC/C=C(\C)CC/C=C(\C)CCC=C(C)C)CC2] | Mertansine-γ-tocotrienol conjugate with a cleavable carbonate bond |
| 9 | 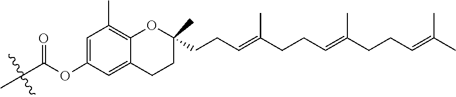 [SMILES: *C(=O)Oc1cc(C)c2c(c1)CC[C@@](C)(CC/C=C(\C)CC/C=C(\C)CCC=C(C)C)O2] | Mertansine-δ-tocotrienol conjugate with a cleavable carbonate bond |
| 10 | 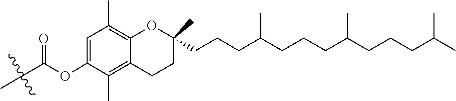 [SMILES: *C(=O)Oc1cc(C)c2c(c1C)CC[C@@](C)(CCCC(C)CCCC(C)CCCC(C)C)O2] | Mertansine-β-tocopherol conjugate with a cleavable carbonate bond |
| 11 | 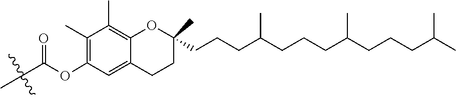 [SMILES: *C(=O)Oc1cc2c(c(C)c1C)O[C@](C)(CCCC(C)CCCC(C)CCCC(C)C)CC2] | Mertansine-γ-tocopherol conjugate with a cleavable carbonate bond |
| 12 | 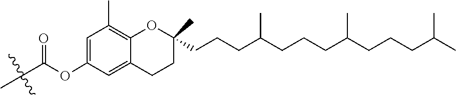 [SMILES: *C(=O)Oc1cc(C)c2c(c1)CC[C@@](C)(CCCC(C)CCCC(C)CCCC(C)C)O2] | Mertansine-δ-tocopherol conjugate with a cleavable carbonate bond |
| 13 | 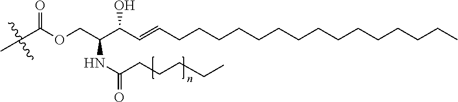 | n = 0 to 10 Mertansine-ceramide conjugate with a cleavable carbonate bond |
| 14 | 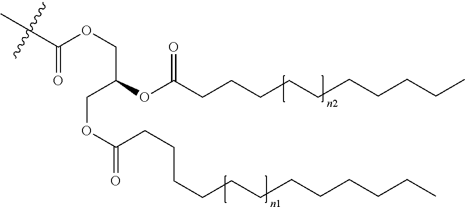 | n1 = 0 to 4 n2 = 0 to 4 Mertansine- diacylglycerol conjugate with a cleavable carbonate bond |

TABLE 2

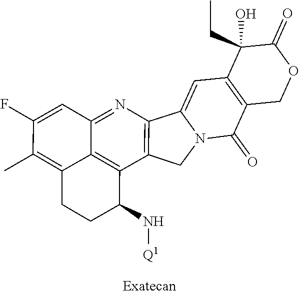

| No. | Q¹ | Trivial Name |
|---|---|---|
| 15 | 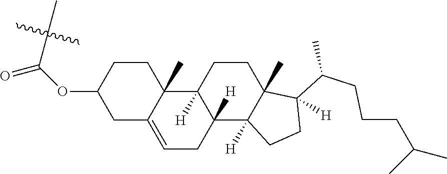 | Exatecan-cholesterol conjugate with a cleavable carbamate bond |
| 16 | 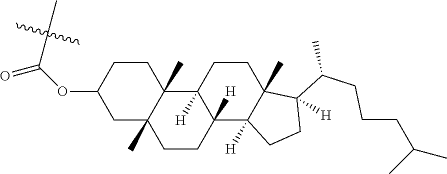 | Exatecan-coprostanol conjugate with a cleavable carbamate bond |
| 17 | 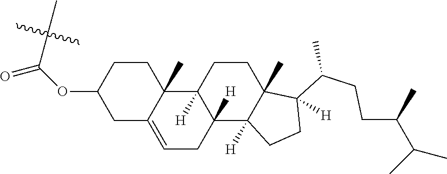 | Exatecan-campesterol conjugate with a cleavable carbamate bond |
| 18 | 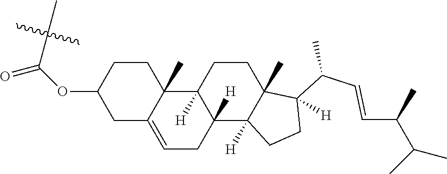 | Exatecan-brassicasterol conjugate with a cleavable carbamate bond |
| 19 | 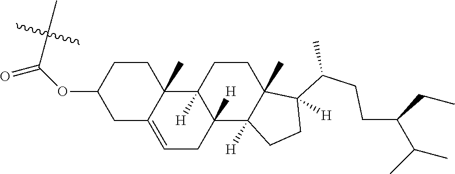 | Exatecan-sitosterol conjugate with a cleavable carbamate bond |
| 20 | 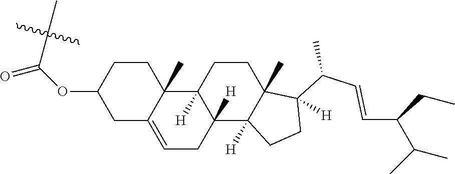 | Exatecan-stigmasterol conjugate with a cleavable carbamate bond |
| 21 | 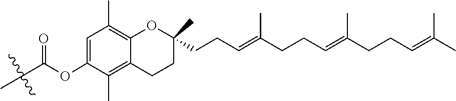 [SMILES: *C(=O)Oc1cc(C)c2c(c1C)CC[C@@](C)(CC/C=C(\C)CC/C=C(\C)CCC=C(C)C)O2] | Exatecan-β-tocotrienol conjugate with a cleavable carbamate bond |
| 22 | 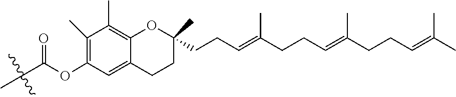 [SMILES: *C(=O)Oc1cc2c(c(C)c1C)O[C@](C)(CC/C=C(\C)CC/C=C(\C)CCC=C(C)C)CC2] | Exatecan-γ-tocotrienol conjugate with a cleavable carbamate bond |
| 23 | 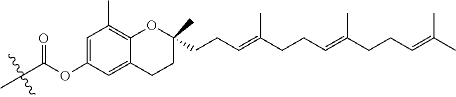 [SMILES: *C(=O)Oc1cc(C)c2c(c1)CC[C@@](C)(CC/C=C(\C)CC/C=C(\C)CCC=C(C)C)O2] | Exatecan-δ-tocotrienol conjugate with a cleavable carbamate bond |
| 24 | 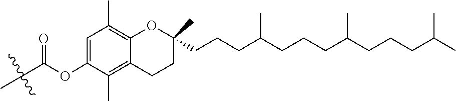 [SMILES: *C(=O)Oc1cc(C)c2c(c1C)CC[C@@](C)(CCCC(C)CCCC(C)CCCC(C)C)O2] | Exatecan-β-tocopherol conjugate with a cleavable carbamate bond |
| 25 | 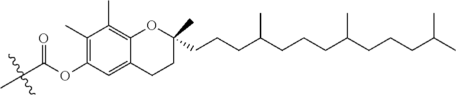 [SMILES: *C(=O)Oc1cc2c(c(C)c1C)O[C@](C)(CCCC(C)CCCC(C)CCCC(C)C)CC2] | Exatecan-γ-tocopherol conjugate with a cleavable carbamate bond |
| 26 | 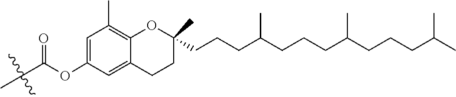 [SMILES: *C(=O)Oc1cc(C)c2c(c1)CC[C@@](C)(CCCC(C)CCCC(C)CCCC(C)C)O2] | Exatecan-δ-tocopherol conjugate with a cleavable carbamate bond |
| 27 | 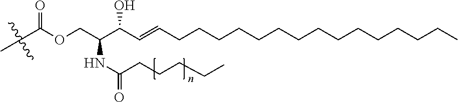 | n = 0 to 10 Exatecan-ceramide conjugate with a cleavable carbamate bond |
| 28 | 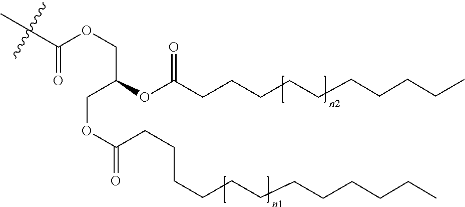 | n1 = 0 to 4 n2 = 0 to 4 Exatecan-diacylglycerol conjugate with a cleavable carbamate bond |

TABLE 3

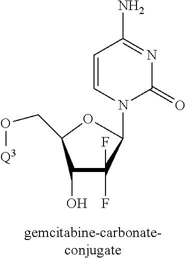

| No. | Q³ | Trivial Name |
|---|---|---|
| 29 | 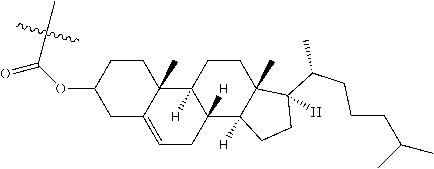 | Gemcitabine-cholesterol conjugate with a cleavable carbonate bond |
| 30 | 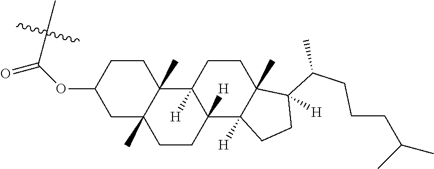 | Gemcitabine-coprostanol conjugate with a cleavable carbonate bond |
| 31 | 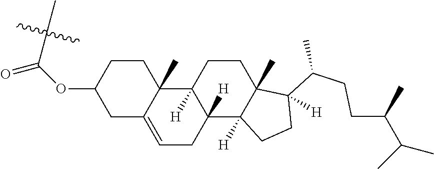 | Gemcitabine-campesterol conjugate with a cleavable carbonate bond |
| 32 | 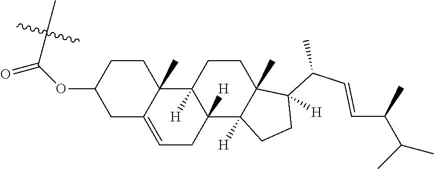 | Gemcitabine- brassicasterol conjugate with a cleavable carbonate bond |
| 33 | 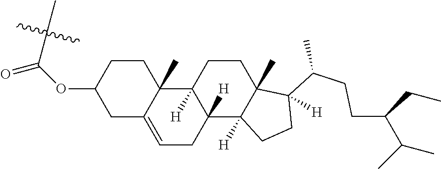 | Gemcitabine-sitosterol conjugate with a cleavable carbonate bond |
| 34 | 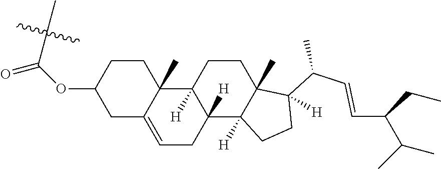 | Gemcitabine-stigmasterol conjugate with a cleavable carbonate bond |
| 35 | 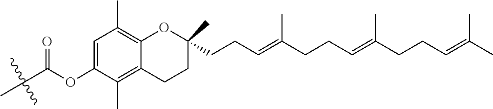 [SMILES: *C(=O)Oc1cc(C)c2c(c1C)CC[C@@](C)(CC/C=C(\C)CC/C=C(\C)CCC=C(C)C)O2] | Gemcitabine-β- tocotrienol conjugate with a cleavable carbonate bond |
| 36 | 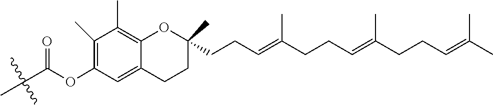 [SMILES: *C(=O)Oc1cc2c(c(C)c1C)O[C@](C)(CC/C=C(\C)CC/C=C(\C)CCC=C(C)C)CC2] | Gemcitabine-γ- tocotrienol conjugate with a cleavable carbonate bond |
| 37 | 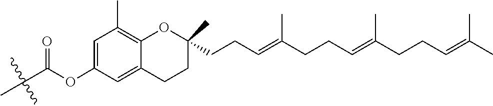 [SMILES: *C(=O)Oc1cc(C)c2c(c1)CC[C@@](C)(CC/C=C(\C)CC/C=C(\C)CCC=C(C)C)O2] | Gemcitabine-δ- tocotrienol conjugate with a cleavable carbonate bond |
| 38 | 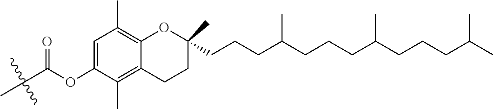 [SMILES: *C(=O)Oc1cc(C)c2c(c1C)CC[C@@](C)(CCCC(C)CCCC(C)CCCC(C)C)O2] | Gemcitabine-β- tocopherol conjugate with a cleavable carbonate bond |
| 39 | 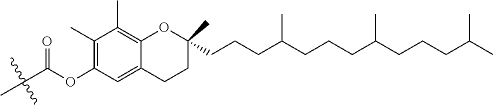 [SMILES: *C(=O)Oc1cc2c(c(C)c1C)O[C@](C)(CCCC(C)CCCC(C)CCCC(C)C)CC2] | Gemcitabine-γ- tocopherol conjugate with a cleavable carbonate bond |
| 40 | 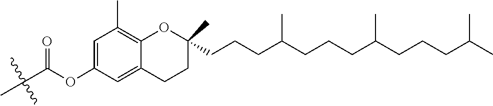 [SMILES: *C(=O)Oc1cc(C)c2c(c1)CC[C@@](C)(CCCC(C)CCCC(C)CCCC(C)C)O2] | Gemcitabine-δ- tocopherol conjugate with a cleavable carbonate bond |
| 41 | 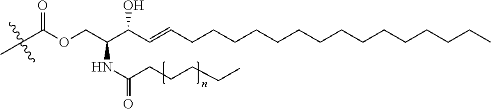 | n = 0 to 10 Gemcitabine-ceramide conjugate with a cleavable carbonate bond |
| 42 | 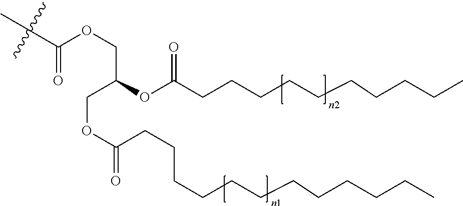 | n1 = 0 to 4 n2 = 0 to 4 Gemcitabine-diacyl- glycerol conjugate with a cleavable carbonate bond |

TABLE 4

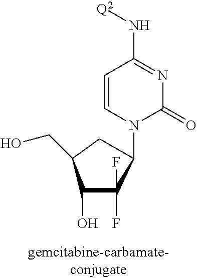

| No. | Q² | Trivial Name |
|---|---|---|
| 43 | 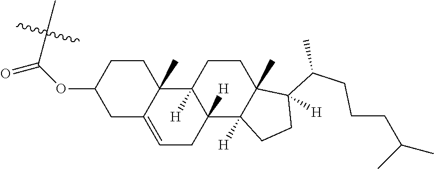 | Gemcitabine-cholesterol conjugate with a cleavable carbamate bond |
| 44 | 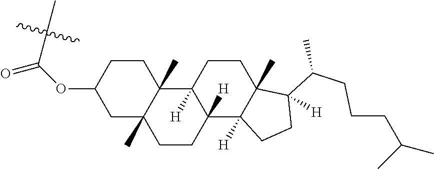 | Gemcitabine-coprostanol conjugate with a cleavable carbamate bond |
| 45 | 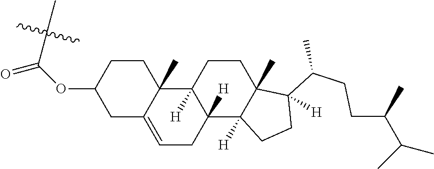 | Gemcitabine-campesterol conjugate with a cleavable carbamate bond |
| 46 | 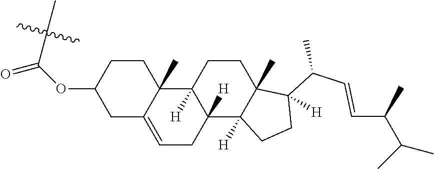 | Gemcitabine- brassicasterol conjugate with a cleavable carbamate bond |
| 47 | 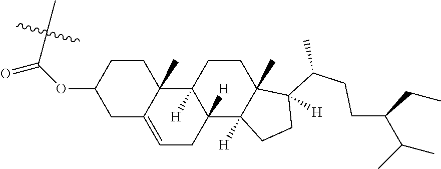 | Gemcitabine-sitosterol conjugate with a cleavable carbamate bond |
| 48 | 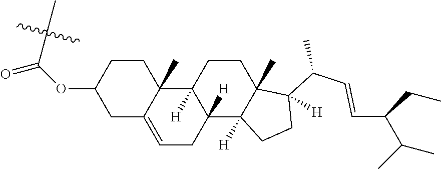 | Gemcitabine-stigmasterol conjugate with a cleavable carbamate bond |
| 49 | 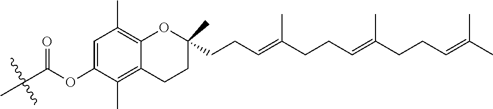 [SMILES: *C(=O)Oc1cc(C)c2c(c1C)CC[C@@](C)(CC/C=C(\C)CC/C=C(\C)CCC=C(C)C)O2] | Gemcitabine-β- tocotrienol conjugate with a cleavable carbamate bond |
| 50 | 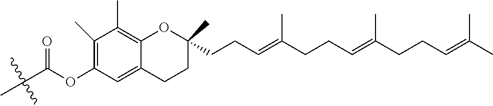 [SMILES: *C(=O)Oc1cc2c(c(C)c1C)O[C@](C)(CC/C=C(\C)CC/C=C(\C)CCC=C(C)C)CC2] | Gemcitabine-γ- tocotrienol conjugate with a cleavable carbamate bond |
| 51 | 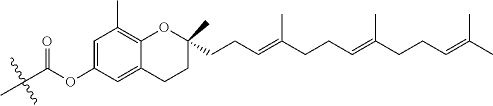 [SMILES: *C(=O)Oc1cc(C)c2c(c1)CC[C@@](C)(CC/C=C(\C)CC/C=C(\C)CCC=C(C)C)O2] | Gemcitabine-δ- tocotrienol conjugate with a cleavable carbamate bond |
| 52 | 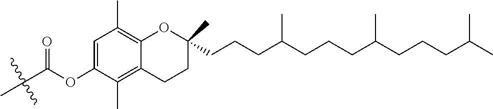 [SMILES: *C(=O)Oc1cc(C)c2c(c1C)CC[C@@](C)(CCCC(C)CCCC(C)CCCC(C)C)O2] | Gemcitabine-β- tocopherol conjugate with a cleavable carbamate bond |
| 53 | 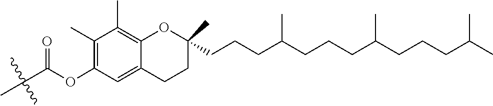 [SMILES: *C(=O)Oc1cc2c(c(C)c1C)O[C@](C)(CCCC(C)CCCC(C)CCCC(C)C)CC2] | Gemcitabine-γ- tocopherol conjugate with a cleavable carbamate bond |
| 54 | 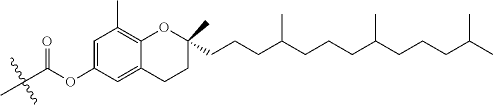 [SMILES: *C(=O)Oc1cc(C)c2c(c1)CC[C@@](C)(CCCC(C)CCCC(C)CCCC(C)C)O2] | Gemcitabine-δ- tocopherol conjugate with a cleavable carbamate bond |
| 55 | 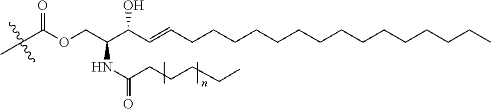 | n = 0 to 10 Gemcitabine-ceramide conjugate with a cleavable carbamate bond |
| 56 | 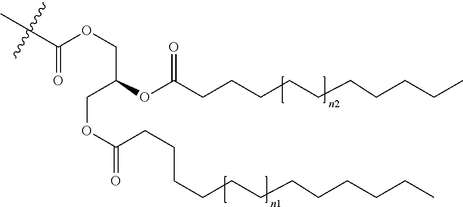 | n1 = 0 to 4 n2 = 0 to 4 Gemcitabine-diacyl- glycerol conjugate with a cleavable carbamate bond |

TABLE 5

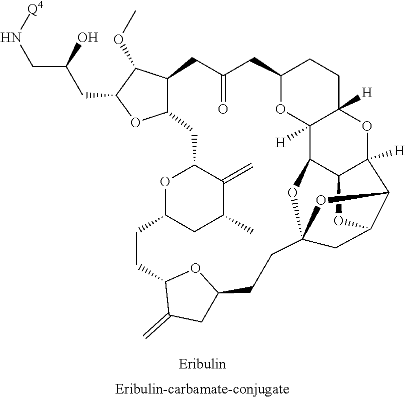

| No. | Q⁴ | Trivial Name |
|---|---|---|
| 57 | 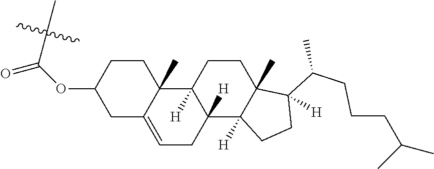 | Eribulin-cholesterol conjugate with a cleavable carbamate bond |
| 58 | 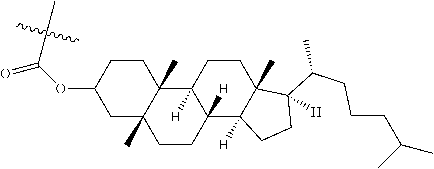 | Eribulin-coprostanol conjugate with a cleavable carbamate bond |
| 59 | 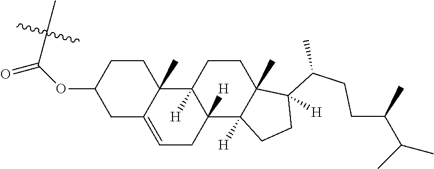 | Eribulin-campesterol conjugate with a cleavable carbamate bond |
| 60 | 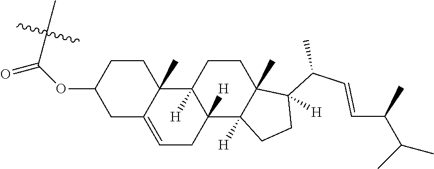 | Eribulin-brassicasterol conjugate with a cleavable carbamate bond |
| 61 | 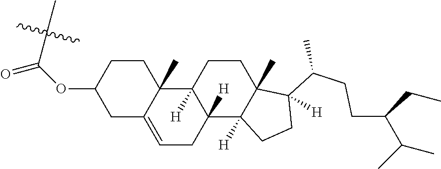 | Eribulin-sitosterol conjugate with a cleavable carbamate bond |
| 62 | 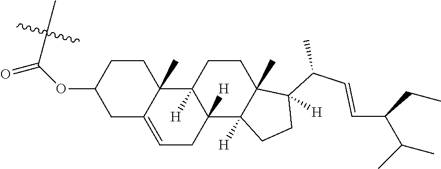 | Eribulin-stigmasterol conjugate with a cleavable carbamate bond |
| 63 | 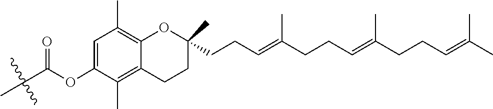 [SMILES: *C(=O)Oc1cc(C)c2c(c1C)CC[C@@](C)(CC/C=C(\C)CC/C=C(\C)CCC=C(C)C)O2] | Eribulin-β-tocotrienol conjugate with a cleavable carbamate bond |
| 64 | 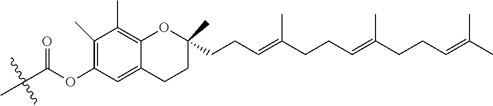 [SMILES: *C(=O)Oc1cc2c(c(C)c1C)O[C@](C)(CC/C=C(\C)CC/C=C(\C)CCC=C(C)C)CC2] | Eribulin-γ-tocotrienol conjugate with a cleavable carbamate bond |
| 65 | 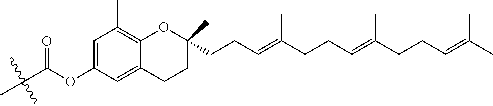 [SMILES: *C(=O)Oc1cc(C)c2c(c1)CC[C@@](C)(CC/C=C(\C)CC/C=C(\C)CCC=C(C)C)O2] | Eribulin-δ-tocotrienol conjugate with a cleavable carbamate bond |
| 66 | 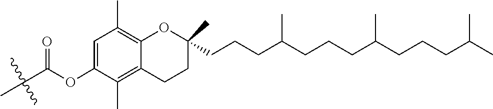 [SMILES: *C(=O)Oc1cc(C)c2c(c1C)CC[C@@](C)(CCCC(C)CCCC(C)CCCC(C)C)O2] | Eribulin-β-tocopherol conjugate with a cleavable carbamate bond |
| 67 | 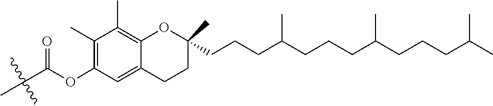 [SMILES: *C(=O)Oc1cc2c(c(C)c1C)O[C@](C)(CCCC(C)CCCC(C)CCCC(C)C)CC2] | Eribulin-γ-tocopherol conjugate with a cleavable carbamate bond |
| 68 | 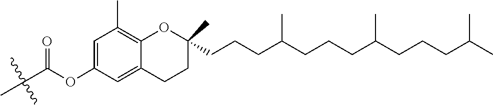 [SMILES: *C(=O)Oc1cc(C)c2c(c1)CC[C@@](C)(CCCC(C)CCCC(C)CCCC(C)C)O2] | Eribulin-δ-tocopherol conjugate with a cleavable carbamate bond |
| 69 | 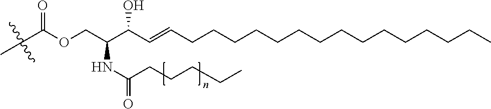 | n = 0 to 10 Eribulin-ceramide conjugate with a cleavable carbamate bond |
| 70 | 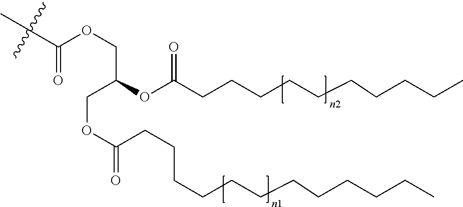 | n1 = 0 to 4 n2 = 0 to 4 Eribulin-diacyl- glycerol conjugate with a cleavable carbamate bond |

TABLE 6

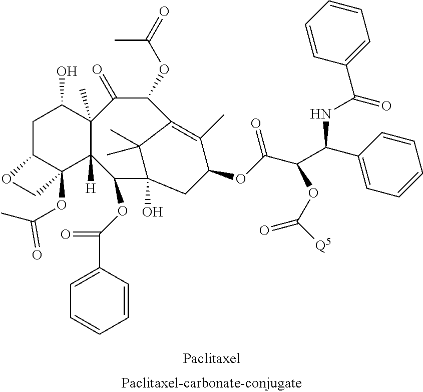

| No. | Q⁵ | Trivial Name |
|---|---|---|
| 71 | 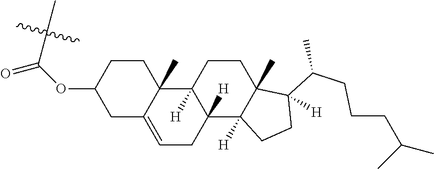 | Paclitaxel-cholesterol conjugate with a cleavable carbonate bond |
| 72 | 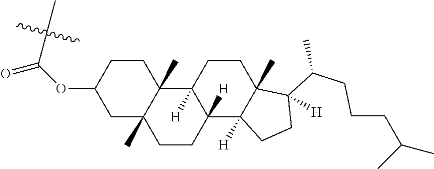 | Paclitaxel-coprostanol conjugate with a cleavable carbonate bond |
| 73 | 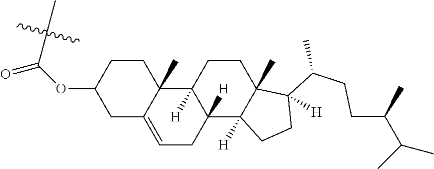 | Paclitaxel-campesterol conjugate with a cleavable carbonate bond |
| 74 | 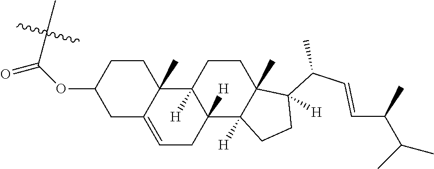 | Paclitaxel- brassicasterol conjugate with a cleavable carbonate bond |
| 75 | 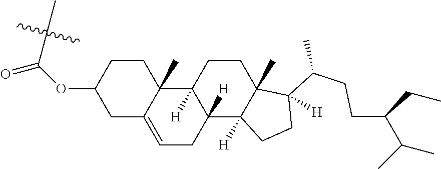 | Paclitaxel-sitosterol conjugate with a cleavable carbonate bond |
| 76 | 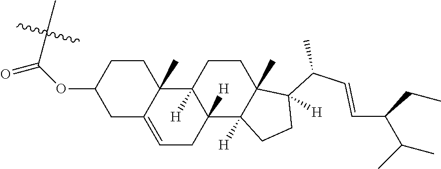 | Paclitaxel-stigmasterol conjugate with a cleavable carbonate bond |
| 77 | 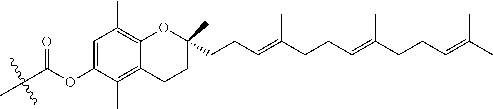 [SMILES: *C(=O)Oc1cc(C)c2c(c1C)CC[C@@](C)(CC/C=C(\C)CC/C=C(\C)CCC=C(C)C)O2] | Paclitaxel-β-tocotrienol conjugate with a cleavable carbonate bond |
| 78 | 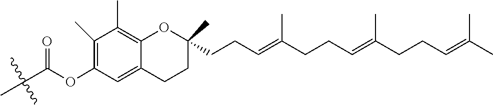 [SMILES: *C(=O)Oc1cc2c(c(C)c1C)O[C@](C)(CC/C=C(\C)CC/C=C(\C)CCC=C(C)C)CC2] | Paclitaxel-γ-tocotrienol conjugate with a cleavable carbonate bond |
| 79 | 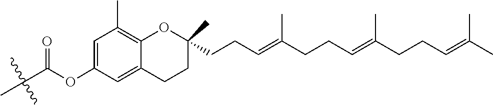 [SMILES: *C(=O)Oc1cc(C)c2c(c1)CC[C@@](C)(CC/C=C(\C)CC/C=C(\C)CCC=C(C)C)O2] | Paclitaxel-δ-tocotrienol conjugate with a cleavable carbonate bond |
| 80 | 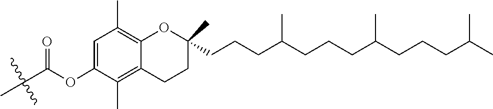 [SMILES: *C(=O)Oc1cc(C)c2c(c1C)CC[C@@](C)(CCCC(C)CCCC(C)CCCC(C)C)O2] | Paclitaxel-β-tocopherol conjugate with a cleavable carbonate bond |
| 81 | 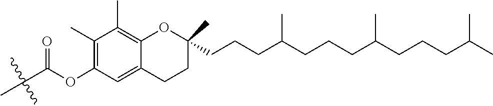 [SMILES: *C(=O)Oc1cc2c(c(C)c1C)O[C@](C)(CCCC(C)CCCC(C)CCCC(C)C)CC2] | Paclitaxel-γ-tocopherol conjugate with a cleavable carbonate bond |
| 82 | 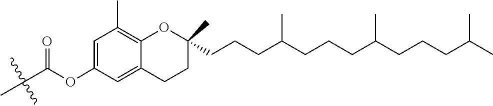 [SMILES: *C(=O)Oc1cc(C)c2c(c1)CC[C@@](C)(CCCC(C)CCCC(C)CCCC(C)C)O2] | Paclitaxel-δ-tocopherol conjugate with a cleavable carbonate bond |
| 83 | 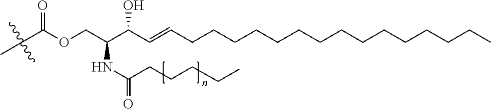 | n = 0 to 10 Paclitaxel-ceramide conjugate with a cleavable carbonate bond |
| 84 | 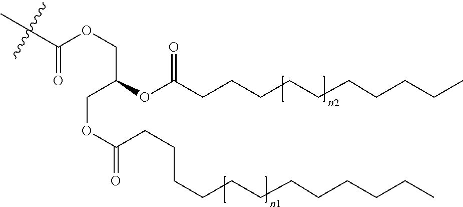 | n1 = 0 to 4 n2 = 0 to 4 Paclitaxel-diacyl- glycerol conjugate with a cleavable carbonate bond |

In addition to mertansine, exatecan, gemcitabine, eribulin, and paclitaxel-containing conjugates like the examples show in Tables 1-6, other conjugates useful herein include an anchor compound and a chemotherapeutic agent linked by a cleavable linker. Suitable other chemotherapeutic agents include, by way of example only, bendamustine (a conjugate linked by a carboxyl ester), chlorozotocin (a conjugate linked by a carbonate bond at the methylene-OH group), capecitabine (a conjugate linked by a carbonate bond), melphanine (a conjugate linked by a carbamate or an ester bond), streptzotocin (a conjugate linked by a carbonate bond at the methylene-OH group), mitoxantrone (a conjugate linked by a carbamate or an ester bond), hydroxy camptothecin (e.g., 7-ethyl-hydroxy camptothecin which forms a conjugate linked by an ester bond), troxacitabine (a conjugate linked by a carbamate or an ester bond), vincristine (a conjugate linked by an ester bond), sirolimus (a conjugate linked by an ester bond), tubulysin A (a conjugate linked by a carbonate or an ester bond), docetaxel (a conjugate formed by an ester bond or a carbonate bond), and cytarabine (a conjugate linked by a carbonate or a carbamate bond).

In some embodiments, the formulation further comprises an amphiphilic, alpha-helical peptide selected from an amino acid sequence as provided by SEQ ID NO: 25, SEQ ID NO: 28, SEQ ID NO: 34, SEQ ID NO: 35, SEQ ID NO:36.

In some embodiments, there is provided a method for preparing a solution of an amphiphilic, alpha-helical peptide selected from an amino acid sequence as provided by SEQ ID NO: 25, SEQ ID NO: 28, SEQ ID NO: 34, SEQ ID NO: 35, SEQ ID NO:36 and combinations thereof in an aqueous-ethanol co-solvent, which method comprises:
a) combining in any order from about 50 to about 90 weight percent ethanol and from about 5 to about 50 weight percent water and about 0 to about 7.5 weight percent benzyl alcohol as needed depending on the character of the chemotherapeutic agent;
b) adding from about 2 to about 10 weight percent of an acid based on the weight of a) above;
c) adding from about 1 to about 2.5 weight percent of an amphiphilic, alpha-helical peptide selected from an amino acid sequence as provided by SEQ ID NO:25, SEQ ID NO:28, SEQ ID NO:34, SEQ ID NO:35 and SEQ ID NO:36 based on the total weight of the solvent solution of b) above; and
d) stirring until a clear solution is obtained.

In a particular embodiment where the conjugate is an exatecan conjugate, the amount of water is about 5 weight percent and sufficient benzyl alcohol is added to solubilize all of the components in Composition 1 as shown in Example 5.

In some embodiments, there is provided a method for preparing a composition suitable for use in preparing C-m-HDLs as described herein which method comprises: preparing a composition designated as “Composition 1”:
a) by combining in any order:
   i) ethanol and water to form a cosolvent having from about 50 to about 90 weight percent ethanol and from about 7.5 to about 50 weight percent water;
   ii) about 2 to 10 weight percent of an acid based on the weight of the cosolvent;
   iii) about 1 to 2.5 weight percent of a peptide of SEQ ID NO:25, SEQ ID NO: 28, SEQ ID NO:34; SEQ ID NO:35 or SEQ ID NO:36 based on the weight of the cosolvent of ii);
   wherein the total amount of i), ii) and iii) equals 100 weight percent, and stirring the composition until the solution is clear evidencing that the peptide is completely dissolved; and
b) combining the solution generated in a) with from about 3 to about 6 weight percent of a lipid compositions based on the weight of the solution of a) wherein said lipid composition comprises:
   iv) about 50 to 70 weight percent of an additional lipid component such as 1-palmitoyl-2-oleoyl-phosphatidylcholine (POPC) (commercially available from Lipoid, Newark, New Jersey, USA)
   v) about 18 to about 30 weight percent of sphingomyelin (SM) (commercially available from Lipoid, Newark, New Jersey, USA), and
   vi) about 12 to about 35 weight percent of a conjugate as described above;
   and further wherein the combined solution generated in a) and the solution of b) equals 100 weight percent; and
c) stirring as necessary to provide for a clear solution, wherein the clear solution is designated as “Composition 1.”

In some embodiments, only half of the water to be used is combined with the components in a) and b). The resulting solution is then stirred until the components are dissolved. At that time, the remaining half of the water is added.

In some embodiments, there is provided a method for reducing the size of the C-m-HDLs which method comprises:
a) combining water with from about 2 to about 6 weight percent of a disaccharide and mixing until homogeneous, wherein the homogeneous mixture is “Composition 2”;
b) adding Composition 2 to a first loading chamber of a three-chamber mixing device which comprises two separate loading chambers and a single reaction chamber;
c) adding Composition 1 into the other loading chamber of said two loading chambers in the device; and
d) chaotically mixing the two compositions into the reaction chamber under a controlled flow rate ratio of from about 4:1 to about 10:1 of Composition 2 to Composition 1 at a temperature of from about 30° to about 70° C. while maintaining the flow from each chamber until mixing is complete while maintaining a temperature of from about 30° to about 70° C.; thereby providing a suspension of C-m-HDLs having a mean number average diameter for the nanoparticles of about 11.5 nanometers to about 14 nanometers, and more preferably about 12 nanometers to about 13 or about 13.5 nanometers.

In some embodiments, the flow rate ratio of Composition 2 and Composition 1 into the chaotically mixing reaction chamber is about 7:1.

In some embodiments, the flow rate of Composition 2 into the chaotically mixing reaction chamber is about 3.5 mL/min and the flowrate of Composition 1 into the chaotically mixing reaction chamber is about 0.5 mL/min.

In some embodiments, the disaccharide is sucrose and the conjugate includes paclitaxel, and the proportions of components used are selected to provide a final composition target of 5 mg/mL of paclitaxel equivalents in the about 4% or the about 5% sucrose solution.

In some embodiments, the C-m-HDLs provided herein have a distribution of particle sizes wherein at least about 70% of said particles are within plus/minus about 3 nanometers.

In some embodiments, the method described above further comprises removing at least a portion of the ethanol and acetic acid by either dialysis or tangential flow filtration (TFF).

In some embodiments, the resulting nanosuspension (sometimes referred to as “solution”) is sterile filtered preferably using a 0.2 micron filter.

In some embodiments, the method for reducing the size of C-m-HDLs further comprises lyophilizing the composition. Such lyophilization and subsequent reconstitution does not materially alter the physical dimensions of the C-m-HDLs as shown in FIG. 9.

In some embodiments, the amount of disaccharide in the lyophilized composition ranges from about 10 weight percent to about 500 weight percent disaccharide based on the weight of the C-m-HDLs.

In some embodiments, the amount of disaccharide in the final composition ranges from about 20 weight percent to about 200 weight percent disaccharide based on the weight of the C-m-HDLs.

In some embodiments, the disaccharide is selected from sucrose, lactose, maltose, trehalose, cellobiose and lactulose.

In some embodiments, the disaccharide is sucrose.

In some embodiments, the average diameter size of the C-m-HDLs after synthesis is reduced by at least about 15%, or by about 20%, or by about 25% by the presence of from about 2 weight percent to about 6 weight percent disaccharide in Composition 2 described above as compared to the average particle size of the C-m-HDLs after synthesis in the absence of a disaccharide. The average particle size of the C-m-HDLs described herein corresponds substantially to that found after synthesis as well as that found after lyophilization and reconstitution evidencing that lyophilization in the presence of a disaccharide causes no material change in particle size.

In some embodiments, the anchor moiety is exemplified but not limited to cholesterol, β-tocotrienol, γ-tocotrienol, δ-tocotrienol, coprostanol, plant sterols, (β-sitosterol, sitostanol, stigmasterol, stigmastanol, campesterol, brassicasterol), ergosterol, retinol, cholecalciferol, ergocalciferol, β-tocopherol, γ-tocopherol, and δ-tocopherol.

In some embodiments, the conjugate used in preparing the C-m-HDLs described herein is cholesterol, δ-tocotrienol or δ-tocopherol each of which is covalently attached to a chemotherapeutic agent (e.g., paclitaxel) via a cleavable bond.

In some embodiments, there is provide a method for treating a patient with a disorder mediated at least in part by the overexpression of SR-BI which method comprises administering to said patient an effective amount of a composition comprising C-m-HDLs as described herein.

In some embodiments, the disorder is a solid mass tumor that overexpresses SR-BI.

In some embodiments, the disorder is a blood borne cancer that overexpresses SR-BI.

In some embodiments, the solid mass tumor is selected from breast cancer (including triple negative breast cancer), bladder cancer, gastrointestinal cancers, head and neck cancers, neuroblastoma, non-small-cell lung cancer, ovarian cancer, pancreatic cancer, prostate cancer, stomach cancer, kidney cancer, and cervical cancer.

In some embodiments, the blood borne cancer is leukemia, multiple myeloma, lymphoma, and the like.

In some embodiments, an exemplary aqueous stock solution prepared by reconstitution of the lyophilized formulation is used to provide a pharmaceutical composition that contains a population of C-m-HDLs comprising a paclitaxel-δ-tocotrienol conjugate linked together by a carbonate bond, wherein the reconstituted composition contains about 5 mg/mL of paclitaxel equivalents in C-m-HDLs.

In some embodiments there is provided a pharmaceutical composition comprising a pharmaceutically acceptable excipient and a population of C-m-HDLs which composition comprises:
a) water;
b) a disaccharide;
c) a population of C-m-HDLs wherein said C-m-HDLs in said population comprise:
   i) an amphiphilic, alpha-helical peptide wherein said peptide forms an HDL mimetic structure on said C-m-HDLs;
   ii) one or both of sphingomyelin and phosphatidyl choline, and optionally additional lipid(s) or phospholipid(s); and
   iii) a conjugate comprising an anchor moiety and a paclitaxel that are releasably attached to each other through a cleavable bond;
wherein the C-m-HDLs in the population comprise a hydrophilic exterior surface and a hydrophobic core; and
further wherein the C-m-HDLs in the population have an average particle diameter of from about 11.5 nanometers to about 14 nanometers as measured by dynamic light scattering.

In some embodiments, there is provided a pharmaceutical composition comprising a pharmaceutically acceptable excipient and a population of C-m-HDLs which comprise:
a) water;
b) a disaccharide;
c) a population of C-m-HDLs which population comprises:
   i) an amphiphilic, alpha-helical peptide that comprises an amino acid sequence of any one or more of SEQ ID NO:1 through SEQ ID NO:36;
   ii) one or both of sphingomyelin and phosphatidyl choline, and optionally additional lipid(s) or phospholipid(s); and
   iii) a conjugate comprising an anchor moiety and a paclitaxel that are releasably attached to each other through a cleavable bond;
wherein the C-m-HDLs in the population comprise a hydrophilic exterior surface and a hydrophobic core; and
further wherein the C-m-HDLs in the population have an average particle diameter of from about 11.5 nanometers to about 14 nanometers as measured by dynamic light scattering.

In some embodiments, there is provided a pharmaceutical composition comprising an aqueous composition suitable for intravenous injection which composition comprises sterile water, sucrose and an effective amount of a population of C-m-HDLs which itself comprises:
(a) an amphiphilic, alpha-helical peptide that comprises an amino acid sequence of SEQ ID NO:25, SEQ ID NO:28, SEQ ID NO:34, SEQ ID NO:35, or SEQ ID NO:36,
(b) one or both of sphingomyelin and phosphatidyl choline, and optionally additional lipid(s) including phospholipid(s); and
(c) a conjugate comprising paclitaxel and a lipid which is absorbable by SR-BI,
wherein the lipid is releasably attached to paclitaxel through a carbonate bond;
wherein said C-m-HDLs comprises a hydrophilic exterior surface and a hydrophobic core comprising the conjugate;
wherein each of the C-m-HDLs in the population has an average particle diameter of about 11.5 to about 14 nanometers, as measured by dynamic light scattering.

In some embodiments, the population of C-m-HDLs in said pharmaceutical composition has an average particle diameter of about 12.0 to about 13.5 nanometers, as measured by dynamic light scattering.

In some embodiments, at least about 65% of the C-m-HDLs in said population have a diameter that is within plus/minus about 2 nanometers of the average particle diameter.

In some embodiments, at least about 70% of the C-m-HDLs in said population have a diameter that is within plus/minus about 2 nanometers of the average particle diameter.

In some embodiments, at least about 75% of the C-m-HDLs in said population have a diameter that is within plus/minus about 2 nanometers of the average particle diameter.

In some embodiments, at least about 80% of the C-m-HDLs in said population have a diameter that is within plus/minus about 2 nanometers of the average particle diameter.

In some embodiments, the conjugate used in preparing the C-m-HDLs described herein comprises an anchor group and a chemotherapeutic agent selected from mertansine, exatecan, gemcitabine, eribulin, bendamustine, chlorozotocin, melphalan, streptzotocin, mitoxantrone, hydroxy camptothecin, 7-ethyl-hydoxycamptothecin, troxacitabine, sirolimus, tubulysin A, docetaxel, cabazitaxel, dasatinib, capivasertib, filanesib, ispinesib, and cytarabine.

### BRIEF DESCRIPTION OF THE FIGURES

FIG. 1 illustrates a proposed organization of a m-HDL as described herein. In the example of FIG. 1, compound 20 is also referred to herein as Conjugate 20; peptide refers to a peptide which is any one of SEQ ID NOs 1-36, and phospholipid refers to POPC as defined herein.

FIG. 2 compares the average diameters of C-m-HDLs using different concentrations of sucrose during synthesis with sucrose concentrations of from about 3 to about 5 weight percent in water being most effective in reducing C-m-HDLs particle size.

FIG. 3 provides a gel permeation chromatography that utilizes peptides of a known size against the lyophilized nanoparticle of Example 3 (Lyophilizate-20) to assess the average size of the C-m-HDLs described herein.

FIG. 4 is a schematic of the 3-chamber mixing and reaction apparatus recited in Example 1.

FIG. 5 provides further evidence as to the average diameter of the C-m-HDLs of this disclosure. Specifically, in FIG. 5, the use of 5% sucrose during the synthesis of the C-m-HDLs results in an average diameter of about 13 nm measured by DLS and a tight distribution profile whereas the C-m-HDLs made by the same protocol but in the absence of sucrose showed a characteristically larger average diameter which, in addition, has a much broader distribution.

FIG. 6 illustrates data from a literature reference showing the extent of plasma half-life of comparative nanoparticles based on their size.

FIG. 7 illustrates data from a literature reference showing the extent of depth of tumor penetration of comparative nanoparticles based on their size.

FIG. 8 illustrates data from a literature reference showing the extent of duration of tumor exposure of comparative nanoparticles based on their size.

FIG. 9 illustrates that the average diameter of C-m-HDLs before lyophilization and after lyophilization and reconstitution remain substantially the same.

FIG. 10 provides data demonstrating the lack of adrenal toxicity arising from administration of paclitaxel or of C-m-HDL loaded with Conjugate 20.

FIG. 11 illustrates a graph comparing concentration-dependent growth inhibition of SK-OV-3 human ovarian cancer cells in culture by a reconstituted formulation of a lyophilized compositions of C-m-HDLs using SEQ ID NO: 25 as the peptide and conjugate 20 or conjugate 30 for comparison purposes.

FIG. 12A shows SK-OV-3 growth inhibition by C-m-HDLs prepared with SEQ ID NO: 35 peptide by solvent lyophilization and loaded with a Conjugate 20 (δ-tocotrienol-paclitaxel) provided equivalent results when compared to C-m-HDLs prepared the same except that the anchor lipid is δ-tocopherol-paclitaxel.

FIG. 12B compares SK-OV-3 growth inhibition by C-m-HDLs prepared by solvent lyophilization with SEQ ID NO:35 peptide and loaded with conjugate 20 (δ-tocotrienol-paclitaxel) versus C-m-HDLs loaded with α-tocopherol-paclitaxel. Note that the inactivity of the alpha-tocopherol analog linked to paclitaxel via a carbonate, in the same manner as conjugate 20, is believed to be based on inability of intracellular enzymes to cleave the carbonate bond and perhaps the carbamate bond. However, because the sulfur atom in the thiocarbonate bond of mertansine is significantly larger than oxygen, conjugates of mertansine and alpha tocotrienol/alpha tocopherol should have significantly less steric hinderance and, accordingly, these conjugates are included within in the claimed subject matter.

FIG. 13 shows a comparison of SK-OV-3 growth inhibition results for C-m-HDLs prepared with various peptides by the solvent lyophilization method and loaded with conjugate 20.

FIG. 14 shows a comparison of paclitaxel and C-m-HDL (cmpd 20) (paclitaxel equivalents) inhibition of SK-OV-3 xenograft growth in female, nude mice (athymic).

FIG. 15 illustrates a flow diagram for the synthesis of C-m-HDLs as described herein.

FIG. 16 illustrates the results of Example 21 where a δ-tocotrienol-exatecan conjugate incorporated into C-m-HDLs as described herein was evaluate for its ability to retard ovarian cancer tumor growth.

FIG. 17 illustrates the results of Example 20 where a δ-tocotrienol-mertenasine conjugate incorporated into C-m-HDLs as described herein was evaluate for its ability to retard ovarian cancer tumor growth.

FIGS. 18A through 18N illustrate a set of preferred conjugates.

### DETAILED DESCRIPTION

Disclosed are chemotherapeutic micellular High Density Lipoprotein mimetic nanoparticles (C-m-HDLs) and compositions comprising such C-m-HDLs. These C-m-HDLs are HDL mimetics and, as such, preferentially target cancer cells that overexpress the HDL receptor known as SR-BI. These C-m-HDLs bind to SR-BI and then intracellularly deliver a conjugate that comprises a chemotherapeutic covalently bound to an absorbable lipid (anchor moiety) by a cleavable bond which is cleaved upon cellular absorption. These C-m-HDLs have an average diameter of about 11.5 nanometers to about 14 nanometers measured by DLS, or preferably 12 nanometers to about 13.5 nanometers, and a distribution profile such that at least about 70 percent of the C-m-HDLs are within plus/minus 3 nanometers of that average (i.e., from about 9 nm to about 16.5 nm).

Prior to addressing this disclosure in more detail, the following abbreviations are defined. Abbreviations not defined, have their accepted chemical or biological meaning.

### Abbreviations

ACTH=adrenocorticotropic hormone
DIEA=diisopropylethylamine
DLS=dynamic light scattering
EDTA=ethylenediamine tetraacetic acid
GI₅₀ concentration to achieve 50% inhibition of growth
g=gram
hr=hour
kD=kiloDaltons
kg=kilogram
mg=milligram
C-m-HDL=Chemotherapeutic HDL mimetic micellular nanoparticles comprising a conjugate of a chemotherapeutic and a lipid
min=minute
mL=milliliters
mm−millimeters
mM=millimoles
mTorr=millitorr
MTT=3-(4,5-dimethylthiazol-2-yl)-2,5-diphenyltetrazolium bromide
MWCO=molecular weight cutoff
nm=nanometers
nM=nanomolar
water=unless specified otherwise, water includes sterile water
an average diameter=refers to an average diameter of the C-m-HDLs using the volume average obtained by DLS
PBS phosphate buffered saline (137 mM NaCl, 2.7 mM KCl, 10 mM Na₂HPO₄, and 1.8 mM KH₂PO₄, pH 7.1-7.4)
POPC=1-palmitoyl-2-oleoyl-phosphatidylcholine
PES=polyethersulfone
SEC=size exclusion chromatography
SM=sphingomyelin
SR-BI=scavenger receptor B-type I
Tris=tris(hydroxymethyl) aminomethane
μg=microgram
μL=microliter
μM=micromolar
μm=micron
UV=ultraviolet
v/v=volume to volume
Conjugate 20=paclitaxel-delta-tocotrienol conjugate (aka “compound 20” or “cmpd20”)

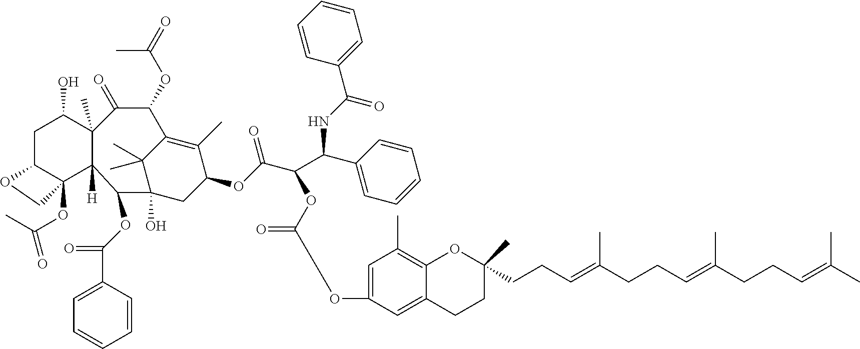 [SMILES: CC(=O)O[C@H]1C(=O)[C@@]2(C)[C@H]([C@H](OC(=O)c3ccccc3)[C@]3(O)C[C@H](OC(=O)[C@H](OC(=O)Oc4cc(C)c5c(c4)CC[C@@](C)(CC/C=C(\C)CC/C=C(\C)CCC=C(C)C)O5)[C@@H](NC(=O)c4ccccc4)c4ccccc4)C(C)=C1C3(C)C)[C@]1(OC(C)=O)CO[C@@H]1C[C@@H]2O]

Conjugate 30 paclitaxel-cholesterol conjugate (aka “compound 30”).

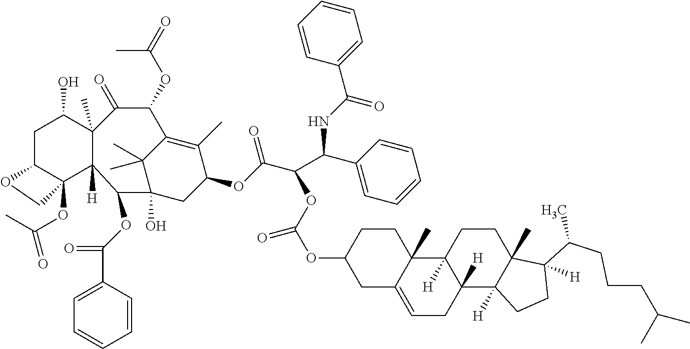

### Terminology

The following provides clarity to certain terms and phrases used in this application. It is understood that terms or phrases used herein that are not defined have their accepted chemical or biological meaning.

The terms “administering” or “administration” or “administer” as used herein means to dispense, provide, and/or apply, and refers to any route of administration that is suitable for use in treating the patient. In some embodiments, intravenous administration is preferably employed to deliver the C-m-HDLs to the patient.

The term “cancer” or “tumor” as used herein refers to both blood-borne cancers as well as solid mass cancers in a subject. SR—BI is enriched in the same type of cancerous tissue as compared to the same type of non-cancerous tissue. In some cases, the comparison is to the adjoining same-type normal tissue of the patient.

The terms “decreasing” or “decrease” or “decreased” or “reducing” or “reduced” or “a reduction” or “inhibiting” as used herein with reference to the C-m-HDLs refer to the extent that the C-m-HDLs disclosed herein impact/treat a cancer in the patient and includes any measurable decrease or complete inhibition of the cancer after being treated with such C-m-HDLs. For example, there may be a decrease of about 1%, 10%, 20%, 30%, 40%, 50%, 60%, 70%, 80%, 90%, 95%, 99%, or more, in the number of tumor cells or in the size of the tumor, or in the occurrence or severity of one or more symptoms of a cancer, in subject receiving C-m-HDLs of the present disclosure, or a composition comprising the same, relative to the number of tumor cells, or in the occurrence or severity of one or more symptoms a cancer, in a subject that has not received C-m-HDLs of the present disclosure, or a composition comprising the same.

The terms “patient” and “subject” as used herein are used herein interchangeably, and refer to any animal (e.g., a mammal, such as a human, a laboratory animal, such as a mouse, rat, rabbit, guinea pig, or other animal models of cancer, or a domesticated animal, such as a dog, cat, or a domesticated animal, for example, sheep, horses, cattle, pigs and goats). A subject in need of treatment, according to the methods described herein, may be one who has been diagnosed with a cancer, such as those described herein, including, without limitation: bladder cancer; breast cancer; gastrointestinal cancers; head and neck cancers; neuroblastoma; non-small-cell lung cancer; ovarian cancer; pancreatic cancer; prostate cancer; stomach cancer, kidney cancer, cervical cancer, leukemia, multiple myeloma, lymphoma, as well as any other cancers that overexpress SR-BI.

The term “pharmaceutically acceptable salts” as used herein is meant to include salts of the peptides or compounds of disclosed herein, which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Non-limiting examples of salts derived from pharmaceutically acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. See, e.g., Berge, S. M., et al, “Pharmaceutical Salts”, Journal of Pharmaceutical Science, 1977, 66, 1-19; the disclosure of which is incorporated herein by reference in its entirety.

The terms “therapeutically effective amount” or “effective amount” or “pharmaceutically effective amount” as used herein refer to a nontoxic but sufficient amount of C-m-HDLs comprising a conjugate wherein a chemotherapeutic agent is covalently linked to an anchor moiety via a cleavable bond to provide the desired biological result, and/or to an amount sufficient to carry out a specifically stated purpose. Included within such definitions are pharmaceutically acceptable salts, or hydrate or solvate thereof, of said C-m-HDLs, or a pharmaceutical composition comprising the same.

As used herein, the terms “treatment” or “treating” or “treatment of” a condition, disease or disorder or symptoms associated with a condition, disease or disorder refer to an approach for obtaining beneficial or desired results, including clinical results. Beneficial or desired clinical results can include, but are not limited to, at least a partial alleviation or partial amelioration of one or more symptoms or conditions; at least a partial diminishment of extent of condition, disorder or disease; at least a partial stabilization of the state of condition, disorder, or disease; prevention or reduced likelihood of developing a condition, disorder, or disease; prevention or reduced likelihood of spreading a condition, disorder, or disease; at least a partial delay or slowing of a condition, disorder, or disease progression; at least partially delay or slowing the progression of a condition, disorder, or disease, or the onset thereof; at least a partial amelioration or palliation of the condition, disorder or disease state, or remission thereof; limiting at least partially one or more of the symptoms of the condition, disorder, or disease state; reducing the severity of the condition, disorder, or disease state and/or any one or more symptoms associated thereof; at least partially relieving the pain associated with and/or caused by the condition, disorder or disease state. As stated above, result of the treatment can be at least partial or total. Methods for determining the result of a treatment are known in the art.

As used herein, when a range is described, that range includes both the endpoints of the range as well as all numbers in between the end points. For example, “between 1 mg and 10 mg” includes 1 mg, 10 mg and all amounts between 1 mg and 10 mg in increments of 0.1.

As used herein, the singular forms “a”, “an” and “the” are intended to include the plural forms as well, unless the context clearly indicates otherwise.

As used herein, the term “about” when used before a numerical designation, e.g., temperature, time, amount, concentration, and such other, including a range, indicates approximations that may vary by plus (+) or minus (−) 10%, 5%, 1%, or any subrange or a sub value therebetween. In some embodiments, the term “about” when used with regard to a dose amount means that the dose may vary by +/−10%.

As used herein, the term “comprising” or “comprises” is intended to mean that the compositions and methods include the recited elements but not excluding others.

As used herein, the term “consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure.

As used herein, the term “consisting of” shall mean excluding more than trace elements of other ingredients and substantial method steps.

Embodiments defined by each of these transition terms are within the scope of this disclosure.

As used herein, the term “chemotherapeutic” or “chemotherapeutic agent” refers to a compound with known anti-tumor properties when used alone or in combination and which contains a reactive hydroxyl, thiol, amino, or carboxyl group thereby permitting the formation of a conjugated having a cleavable carbonate bond, thiocarbonate bond, carbamate bond, thiocarbamate bond, or an ester, with a lipid. The lipid is one that renders the conjugate absorbable by the SRB-1 receptor. Examples of suitable chemotherapeutic agents include, by way of example only, mertansine, exatecan, gemcitabine, eribulin, bendamustine, chlorozotocin, capecitabine, melphanine, streptzotocin, mitoxantrone, hydroxy camptothecin (e.g., 7-ethyl-hydroxy camptothecin), troxacitabine, vincristine, sirolimus, docetaxel, and cytarabine. In some cases, such as with exatecan, the amount of water used in Composition 1 is reduced to about 3 to 5 percent water with benzyl alcohol being added in an amount sufficient to solubilize all of the components.

As used herein, the term “conjugate” refers to a chemotherapeutic agent that is bound to an anchor moiety by a cleavable bond wherein the resulting conjugate that is absorbable into tumor cells such as by SR-BI. In some embodiments, the anchor moiety is recognized by SR-BI on tumor cells and then absorbed into the tumor cell leaving the C-m-HDLs with little to no remaining conjugate. The anchor moiety is bound to the chemotherapeutic agent by a cleavable covalent bond such as a carbonate, a carbamate, an ester, and other cleavable bonds that are severed by intracellular enzymes.

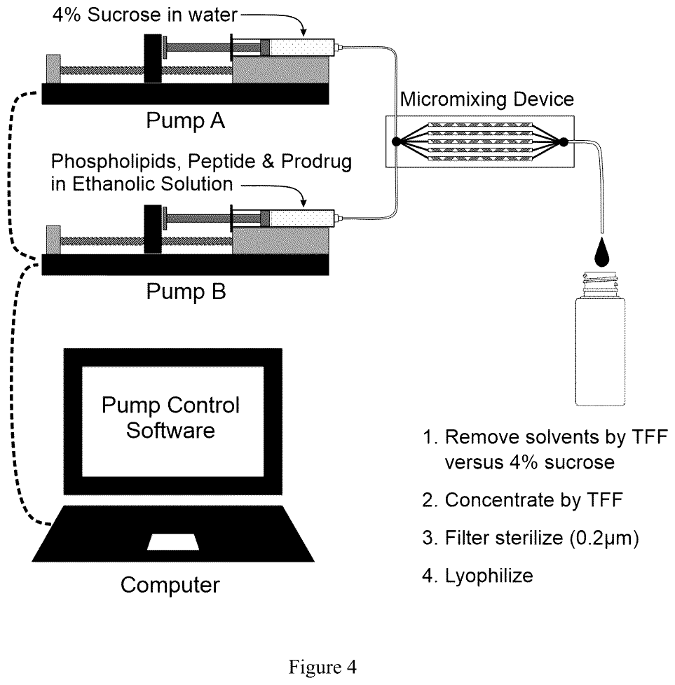

As used herein, “Composition 1” (aka “Composition B”) refers to a stirred and filtered composition comprising all of the components necessary to form the C-m-HDLs described herein except for the sterile water containing about 2 to about 6 weight percent of a disaccharide, and in some examples about 3 to about 6 weight percent of a disaccharide. During synthesis, Composition 1 is added to one of the reaction chambers of a three-chamber device described below and illustrated in FIG. 4.

As used herein, the term “an anchor moiety” refers to the lipid portion of a chemotherapeutic conjugate as defined herein wherein the lipid is absorbable into a tumor cell when the C-m-HDL binds to SR-BI and acts as a gateway agent for intracellular absorption of a chemotherapeutic agent attached thereto. The anchor moiety is conjugated to the chemotherapeutic agent by a bond that is cleavable by intracellular enzymes including but not limited to esterases, lipases, and the like. In some embodiments, the anchor moieties provide a gateway for absorption of the entire conjugate into tumor cells via SR-BI. In such cases, the anchor moiety of the conjugate is a lipid that is absorbable by SR-BI thereby directing the conjugate into the cell wherein it undergoes enzymatic decoupling into the standalone lipid and the standalone chemotherapeutic agent. The “anchor” aspect of these lipids means that once a C-m-HDL is captured by SR-BI on a cell surface, the lipid is extracted from the C-m-HDL and absorbed into the cell. Afterwards, the mimetic is released from the receptor. In some embodiments, the anchor moiety is a lipid is exemplified but not limited to cholesterol, β-tocotrienol, γ-tocotrienol, δ-tocotrienol, coprostanol, plant sterols, (β-sitosterol, sitostanol, stigmasterol, stigmastanol, campesterol, brassicasterol), ergosterol, retinol, cholecalciferol, ergocalciferol, β-tocopherol, γ-tocopherol, and δ-tocopherol.

It is understood that there are certain important features of the C-m-HDLs described herein. First, the peptide components of the C-m-HDLs arrange in aggregates around the surface of these nanoparticles which aggregates to form a mimetic to the peptide structure of HDL thereby acting as a ligand for SR-BI that is overexpressed on tumor cells. In turn, the anchor moiety of the conjugate acts as gateway into the tumor cell as it allows a chemotherapeutic agent such as paclitaxel to be absorbed by that cell. Finally, the cleavable bond acts as a releasing agent that liberates the chemotherapeutic agent such as paclitaxel from the conjugate by the action of intracellular enzymes.

As used herein, the term “average diameter” refers to that volume average diameter obtained by Dynamic Light Scattering (DLS) which average is reported in nanometers (nm). Alternatively, the “average diameter” can be extrapolated by size exclusion chromatograph (SEC) as provided in Example 3. When a diameter is provided without reference to the measurement protocol, such measurements are recited as using DLS methods. Where stated as such, SEC and other equivalent methods may be used to determine average particular diameter.

As used herein, the term “diameter” or “size” as it relates to a C-m-HDL means the length along the longest axis of that nanoparticle.

As used herein, the term “disaccharide” refers to two naturally occurring sugars that form a dimeric structure. The arrangement of the two sugars is not critical and either sugar can be the reducing sugar. For example, a disaccharide comprising N-acetylglucose amine and glucose can have either sugar being the reducing sugar.

As used herein, the term “lipid” refers to an amphiphilic compound having a hydrophilic group at one end and a hydrophobic group attached thereto wherein the lipid participates in micelle or liposome formation. In a preferred embodiment, the lipids are represented by formula I:

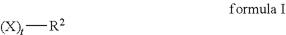

where each X is independently selected from carboxyl, hydroxyl, amino, phosphate, aminophosphate, thiophosphate, and the like; t is one, two or three and each occurrence of X is independently selected from carboxyl, hydroxyl, amino, phosphate, aminophosphate, thiophosphate, and the like; t is no more than the number of carboxyl, hydroxyl, amino, phosphate, aminophosphate or thiophosphate and the like groups on R²; and R² is selected from:
a) a C₁₀-C₃₀ alkyl group and preferably a C₁₆-C₃₀ alkyl group which can be in a straight chain, a branched chained, a cyclic, a multicylic or combinations thereof;
b) a C₁₀-C₃₀ alkenyl group and preferably a C₁₆-C₃₀ alkenyl group either a straight chain, branched chained, cyclic, multicylic or combinations thereof having 1 to 8 double bonds; and
c) a C₁₀-C₃₀ alkynyl group and preferably a C₁₆-C₃₀ alkynyl having from 1 to 4 triple bonds and 0 to 3 triple bonds,
   wherein each of a), b) and c) can contain up to three X groups.

When t is two or three, the second and third X group is located distal to the hydrophobic group in order to maintain the amphiphilic nature of the lipid. For example, triglycerides are fatty acid esters of glycerol and have the general structure of:

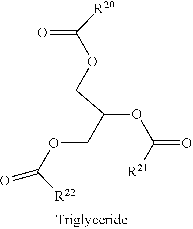

where each of R²⁰, R²¹, and R²² is a fatty acid residue. In this example, t=3 and each of the hydrophilic oxygen atoms are proximate to each other whereas the hydrophobic fatty acid residues extend into space away from the hydrophilic oxygen atoms.

In some embodiments, the anchor moiety employed is absorbable by a tumor cell. In some embodiments, the C-m-HDLs are recognized by and binds to SR-BI thereby allowing the transfer of the conjugate into the cell using the anchor lipid as a gateway into the cell.

As used herein, the term “double bond” refers to a vinyl group in either the cis or trans configuration.

As used herein, the term “amino phosphate” refers to a phosphate group, P (O) 4, where one of the oxygen atoms is replaced by NH or two of the oxygen atoms replaced by NH.

As used herein, the term “thiophosphate” refers to a phosphate group, P (O) 4, where one of the oxygen atoms is replaced by S or two of the oxygen atoms replaced by a single bond SH where the hydrogen is lost during covalently coupling.

As used herein, the term “HDL” refers to high density lipoprotein. In some embodiments, the HDL particles are known to have varying diameters. However, the diameter that provides for highest binding efficiency to SR-BI is reported to have an average diameter of approximately 12 nanometers.

As used herein, the term “C-m-HDLs” refers to HDL mimetic micellar nanoparticles containing a chemotherapeutic, as described herein. Such C-m-HDLs contain a conjugate, also as described herein, which can vary with each population of C-m-HDLs. C-m-HDLs are sometimes referred to herein as “chemotherapeutic nanoparticles” or “chemotherapeutic micellular nanoparticles”.

As used herein, the term “chemotherapeutic equivalents” refers to weight amount of a free chemotherapeutic in the dose of conjugate to be delivered to the subject in need thereof. For example, to achieve a 5 mg/mL of a chemotherapeutic in the population of C-m-HDLs, one merely ascertains the weight of the C-m-HDLs to be delivered, the concentration of the conjugate in the C-m-HDLs, and the weight percentage of chemotherapeutic in the conjugate.

As used herein, the term “amphiphilic” describes a molecule or polymer (e.g. peptide) with affinity for both lipid and aqueous phases due to a conformation in which hydrophilic (water seeking) substituents and hydrophobic (water avoiding) substituents in the molecule or polymer are structurally segregated from one another.

As used herein, the term “lipophilic” describes a substance or part of that substance that distributes preferentially into the hydrophobic domains of lipid-rich particles in aqueous suspension. The lipid-rich particles include lipid micelles, liposomes, lipoproteins, cell membranes and lipid emulsions.

As used herein, the term “micelle” or “micellular” as it relates to the nanoparticles of the C-m-HDLs described herein refers to a multi-molecular structure organized by covalent and non-covalent interactions in an aqueous phase without regard to whether such interactions render the C-m-HDLs water soluble or as a fine water suspension. The C-m-HDLs comprise amphiphilic and hydrophobic molecules which aggregate in such a manner that the hydrophobic domains of molecules are retained in the interior of the C-m-HDLs and, thus, are shielded from the water and the hydrophilic domains at the nanoparticle/micelle-water interface.

As used herein, the term “Aib” or “B” is a three letter or one letter code for the amino acid “alpha-amino isobutyric acid”.

As used herein, the term “dynamic light scattering” or “DLS” refers to a method for determining the average sizes of C-m-HDL particles. Methods of determining average particle sizes, such as DLS, are well known in the art. For example, methods of determining particle size include, without limitation: flow cytometry, electron microscopy, ultracentrifugation, gel filtration, high performance liquid chromatography (HPLC), or any combination thereof. In other embodiments, quasi-elastic light scattering can be used to determine the size of a particle.

Dynamic light scattering (DLS) is the most common analytical technique used for determining nanoparticle size, especially particles that are below 1 μm in size. Evaluation of C-m-HDLs via DLS provides the average diameter and distribution of the C-m-HDL. Moreover, DLS can distinguish whether the C-m-HDLs s are uniformly distributed around one or more particle sizes (unimodal vs. bimodal).

In some embodiments, dynamic light scattering (DLS) can be used to determine the diameter of a C-m-HDL.

In some embodiments, C-m-HDL size refers to the size as determined by photon correlation spectroscopy (PCS). The average size can be expressed as Z average diameter (ZAD) and the polydispersity index (PDI), as determined using photon correlation spectroscopy according to ISO 22412. ISO 22412:2017 specifies the application of DLS to the measurement of average hydrodynamic particle size and the measurement of the size distribution of mainly submicrometer-sized particles, emulsions, or fine bubbles dispersed in liquids.

In some embodiments, ZAD and PDI are determined on the basis of data obtained by dynamic light scattering (DLS). Briefly, a monochromatic and coherent laser light beam illuminates a representative sample for particle size analysis, dispersed in a liquid at a suitable concentration. The light scattered by the particles at a given angle is recorded by a detector (e.g., an avalanche photodiode) whose output is fed to a correlator. Because the dispersed particles are in continuous Brownian motion, the observed scattered intensity fluctuates along the time axis. Therefore, analysis as a function of time of these intensity fluctuations provides information on the motion of the dispersed particles. In a DLS experiment, the time analysis is carried out with a correlator which constructs the time autocorrelation function of the scattered intensity. The decay rate is linked to the translational diffusion coefficient D of the particles. This decay is interpreted in terms of average particle size and polydispersity index by the so-called cumulants method.

For non-interacting spherically shaped particles dispersed in a medium of viscosity n, the diffusion coefficient D is related to the particle hydrodynamic diameter dH by the Stokes-Einstein equation, formula II:

$$D = \frac{k T}{3 π η {d}_{H}} \qquad formula II$$

wherein:
k is the Boltzmann constant,
T the absolute temperature
η the viscosity of the medium.

The cumulants method is a simple method of analyzing the autocorrelation function generated by a DLS experiment. The foregoing calculation is defined in ISO 13321 and ISO 22412. The first two terms of this moments expansion is used in practice, a mean value for the size (z-average size or z-average mean or z-average diameter), and a width parameter known as the polydispersity index (PDI).

The z-average size is an intensity-based calculated value and should not be confused with or directly compared to a mass or number mean value produced by other methods. The calculation is defined in the ISO standards, so all systems that use this calculation as recommended should give comparable results if the same scattering angle is used. The z-average size or z-average mean or z-average diameter used in DLS is a parameter also known as the cumulants mean. It is the primary and most stable parameter produced by the technique. The z-average mean is commonly used in a quality control setting as it is defined in ISO 13321 and more recently ISO 22412 which defines this mean as the “harmonic intensity averaged particle diameter.” The z-average size will only be comparable with the size measured by other techniques if the sample is monomodal (i.e., only one peak), spherical, or near-spherical in shape, monodisperse (i.e., very narrow width of distribution), and the sample is prepared in a suitable dispersant, as the z-average mean size can be sensitive to even small changes in the sample (e.g. the presence of a small proportion of aggregates). It should be noted that the z-average is a hydrodynamic parameter and is therefore only applicable to particles in a dispersion or molecules in solution.

For the purposes herein, the DLS values generated are measured at a temperature of 25° C. This is because changes in the viscosity of the solution containing the nanoparticles alter the refractive index values used to calculate size or diameter. It is well known in the art that viscosity is dependent on the temperature and the concentration of the disaccharide with all other factors being equal. Accordingly, for the purposes herein, the instrument used to calculate DLS values is calibrated using micellular nanoparticles comprising a 5% concentration of sucrose in Composition 2 above, a conjugate comprising paclitaxel and delta tocotrienol releasably bound to each other via a carbonate bond, a temperature of 25° C. and using the amount of the remaining components as per Example 1 below. These micellular nanoparticles gave a DLS value for the diameter of the particles of about 12.2 nm (5% sucrose)+/−1 nm.

In some embodiments, the Polydispersity Index (PDI) is a number calculated from a simple two parameter fit to the correlation data (the cumulants analysis). The PDI is dimensionless and scaled from 0 to 1. The very small values (e.g., 0.05) correspond to highly monodisperse standards. The closer the values to 1, the broader the size distribution of the particles. The PDI of a C-m-HDL formulation is a measure of the heterogeneity of C-m-HDL particles in the formulation.

In some embodiments, the size of C-m-HDLs can be measured by dynamic light scattering using a Malvern ZETASIZER® 3000 or a Malvern ZETASIZER® NANO-ZS. See U.S. patent application Ser. No. 11/520,796 (U.S. Patent Publication No. US20070065499A1), the disclosures of which are incorporated by reference herein in their entireties.

In some embodiments, multi-angle light scattering (MALS) can be used to determine the size of C-m-HDLs. An exemplary description of MALS used to characterize C-m-HDLs is provided in Parot et al., Physical characterization of liposomal drug formulations using multi-detector asymmetrical-flow field flow fractionation. J Control Release. 2020 Apr. 10; 320:495-510, the disclosure of which is incorporated herein in its entirety.

Exemplary methods of DLS are provided in e.g., U.S. Pat. No. 4,927,571, and Hupfeld et al., Liposome size analysis by dynamic/static light scattering upon size exclusion-/field flow-fractionation. J Nanosci Nanotechnol. September-October 2006;6 (9-10): 3025-31. Additional exemplary methods of the foregoing techniques are provided in U.S. Pat. Nos. 10,695,424, and 10,722,466, the disclosures of which are incorporated herein by reference in their entireties.

As used herein, “v/v” or “% v/v” or “volume per volume” refers to the volume concentration of a solution (“v/v” stands for volume per volume). Here, v/v can be used when both components of a solution are liquids. For example, when 50 mL of ingredient X is diluted with 50 ml of water, there will be 50 mL of ingredient X in a total volume of 100 mL; therefore, this can be expressed as “ingredient X 50% v/v.” Percent volume per volume (% v/v) is calculated as follows: (volume of solute (mL)/volume of solution (100 mL)); e.g., % v/v=mL of solute/100 mL of solution.

It is understood that certain liquids when combined will result in a contraction of the final volume as compared to what was added. This phenomenon is especially the case for ethanol and water. In such cases, the percent volume to volume is measured pre-addition and not post-addition.

As used herein, “w/w” or “% w/w” or “weight per weight” or “wt/wt” or “% wt/wt” or “wt %” refers to the weight concentration of a composition or solution, i.e., percent weight in weight (“w/w” stands for weight per weight). Here, w/w expresses the number of grams (g) of a constituent in 100 g of solution or mixture. For example, a mixture consisting of 30 g of ingredient X, and 70 g of water would be expressed as “ingredient X 30% w/w.” Percent weight per weight (% w/w) is calculated as follows: (weight of solute (g)/weight of solution (g))×100; or (mass of solute (g)/mass of solution (g))×100.

As used herein, “w/v” or “% w/v” or “weight per volume” refers to the mass concentration of a solution, i.e., percent weight in volume (“w/v” stands for weight per volume). Here, w/v expresses the number of grams (g) of a constituent in 100 mL of solution. For example, if 1 g of ingredient X is used to make up a total volume of 100 mL, then a “1% w/v solution of ingredient X” has been made. Percent weight per volume (% w/v) is calculated as follows: (Mass of solute (g)/Volume of solution (mL))×100.

As used herein, the term “cleavable bond” or “a covalent cleavable bond” refers to a covalent cleavable bond that is broken or cleaved by intracellular conditions. For example, conjugates described herein are coupled by a cleavable bond which, when exposed to intracellular conditions, is cleaved by conditions encountered in the intracellular milieu such as enzymes including lipases and esterases or by changes in pH such as encountered during endocytosis. In some embodiments, the enzymes employed include lipases or esterases which will cleave ester bonds, thioester bonds, carbonate bonds, thiocarbonate bonds, carbamate bonds, and thiocarbamate bonds. In some embodiments, the cleavable bonds are preferably ester and carbonate bonds.

### Overview

The methods and compositions described herein provide for C-m-HDLs having an average diameter size of about 11.5 nanometers to about 14 nanometers, and preferably about 11.7 to about 13.7 nanometers or, in some embodiments, from about 12 to about 13.5 nanometers. In some embodiments, at least about 65%, or 70%, or 75% or 80% of these particles are within plus/minus 3 nanometers of that range in diameter. In some embodiments, the average diameters for the C-m-HDLs are within plus/minus 2 nanometers or plus/minus 1 nanometer of the average diameter. The use of a disaccharide such as sucrose during synthesis of these nanoparticles results in a smaller average diameter of about 12 to about 13.5 nanometers as opposed to the average diameter of about 17 nanometers obtained by DLS in the absence of a disaccharide during synthesis as described in the '105 Patent. In some embodiments, the distribution of the reduced diameters of such C-m-HDLs is tighter than that generated by the same synthetic methods but not using a disaccharide. As such, the disaccharide is essential to the product and is retained in the composition both before and after lyophilization.

The use of a disaccharide to affect a reduction in the average diameter of these nanoparticles is unexpected as the art has disclosed that the use of sucrose during lyophilization increases the diameter of nanoparticles used in those references. See., e.g., Andreana, et al., Materials (Basel), 16 (3): 1212 (2023); Liversidge, et al., U.S. Pat. No. 5,302,401; and Li, et al., Vaccines, (2023) each of which is incorporated herein by reference in its entirety.

### PALM Technology

Peptide amphiphile lipid micelles (PALM) refers to m-HDLs without the addition of a conjugate containing an anchor group and a chemotherapeutic group. That is to say that PALM does not use a conjugate as described herein. When a conjugate is included, the PALM transitions to a C-m-HDL. The peptide portion of PALM has an amphiphile helical character with one surface being hydrophilic and the other hydrophobic.

In some embodiments, the peptide that composes the PALM is alpha-helical in nature, wherein the peptide comprises a hydrophilic face and a hydrophobic opposite face. In some embodiments, the peptides self-assemble to bundle around a hydrophobic core comprising amphiphiles, wherein the hydrophilic portion extends outward and around the peptide bundle, exposing the hydrophilic ends of the amphiphiles and the hydrophilic face of the peptides to the aqueous environment, while retaining the hydrophobic portion of the amphiphiles and the hydrophobic face of the peptides in the core on the opposite side of the hydrophilic face of the peptide amphiphiles. An exemplary description of PALM is provided in the '105 Patent, the disclosure of which is incorporated herein by reference in its entirety.

### Proposed Mechanism

As mentioned above, SR—BI is a multiligand cellular membrane receptor protein which, in vivo, is the receptor for high density lipoprotein (HDL) and allows for uptake of cholesterol and absorbable lipids into cells. The C-m-HDLs of this disclosure take advantage of SR-BI's HDL binding properties, in order to deliver the chemotherapeutic to a tumor cell: here, the chemotherapeutic is attached to cholesterol or an absorbable lipid via a cleavable bond to form a conjugate, and the C-m-HDLs comprising the conjugate are then delivered in a targeted fashion to such tumors. Upon cellular absorption, the cleavable bond is broken (severed), which subsequently results in the intracellular delivery of paclitaxel or other chemotherapeutic agents into the tumor cell.

The high target specificity of the C-m-HDLs described herein decreases off-site binding to healthy cells thereby reducing overall toxicity while still providing for a significant level of target cell toxicity.

As in any ligand-receptor axis, the receptor's binding site for HDL will correspond to the size of HDL, as found in vivo, in order to facilitate transport into the cells. HDL has been classified into three groups comprising: large, i.e., having an average diameter of about 12 nm; medium, i.e., having an average diameter of about 9 nm; and small, i.e., having an average diameter of about 7.5 nm. Moreover, the binding specificity of HDL to SR-BI is tiered, such that larger, less dense HDL spherical particles bind with greater affinity to SR-BI as compared to smaller, more dense HDL particles.

In some embodiments, it is preferred that the distribution of the C-m-HDLs, around an average particle diameter, should vary minimally. In some embodiments, there is provided a population of nanoparticles as described herein having an average diameter of about 11.5 to about 14 nanometers and having minimal variation in its distribution of nanoparticle diameter. This minimal variation eliminates those particles that are significantly too big or too small and have poor affinity to SR-BI.

In some embodiments, provided herein are methods for preparing such C-m-HDLs where reproducible stoichiometric ratios of the reagents are used during the synthesis of the C-m-HDLs of the present disclosure which provides for consistent average diameter from batch to batch.

Incorporating reproducible stoichiometric ratios of components during synthesis can be challenging. For example, using an amphiphilic peptide can, in some embodiments, result in incomplete solubilization, especially in the context of a water: ethanol cosolvent mixture. Any degree of insolubility renders the exact stoichiometric ratios of the given components to be speculative at best and, is considered a factor in creating a larger variability in the distribution of the resulting nanoparticles.

Similarly, when the distribution about the average diameter of micellar nanoparticles is too broad, then a portion (in some embodiments, a large portion) of the micellar nanoparticles can be either too large or too small to have suitable affinity to SR-BI, or have no affinity at all.

One of the problems solved by this disclosure is that the average diameter of the C-m-HDLs, made without the benefit of methods disclosed herein, are about 17 nanometers when measured by DLS. That size is about 40 to 50% larger than the diameter of HDL particles considered to have the highest affinity SR-BI. Therefore, particles previously disclosed in the art are much larger than those found in vivo, and this difference in size and size distribution are not in line with the size of the active site of SR-BI receptor site and, as such, has an adverse impact on the overall efficacy of these particles. Stated differently, these particles do not have a size that maximizes binding efficiency and, accordingly, a maximum biological activity. However, until the disclosure herein, methods to make smaller m-HDLs that had very strong binding to theSR-BI were not known.

In some embodiments, the methods and compositions of the present disclosure provides for C-m-HDLs having a reproducible number average nanoparticle size of about 11.5 to about 14 nm, wherein at least about 70% of these particles possess about plus/minus 3 nm of that range diameter. In a more particular example, the present disclosure provides for C-m-HDLs having a reproducible average nanoparticle size of about 12 to about 13.5 nm, wherein at least about 70% of these particles possess about plus/minus 3 nm of that average diameter. In some embodiments, the C-m-HDLs of the present disclosure utilize a disaccharide during synthesis. For example, in some embodiments, the micellar nanoparticles of the present disclosure utilize sucrose during synthesis, which is retained in the product solution as well as in the lyophilized product. Likewise, it is contemplated that simple sugars such as glucose, glucosamine, fructose, galactose, and the like could be employed instead of a disaccharide. Likewise, trisaccharides and tetrasaccharides can also be considered for such use.

Surprisingly, the use of a disaccharide results in a reduction in the average diameter of the micellar nanoparticles of the present disclosure. Indeed, the reduction in the average diameter of the C-m-HDLs of the present disclosure is unexpected, as the art has disclosed that the use of sucrose during lyophilization increases the diameter of nanoparticles. See, e.g., Andreana, et al., Materials (Basel), 16 (3): 1212 (2023); Liversidge, et al., U.S. Pat. No. 5,302,401; and Li, et al., Vaccines, (2023); the disclosures which are incorporated herein by reference in their entireties.

Without being limited to any theory, for the C-m-HDLs described herein, the incorporation of a disaccharide occurs during the synthesis of the C-m-HDLs, where the disaccharide is integrated into the methods for producing the unique structure of the C-m-HDLs. This is believed to be different from situations where a disaccharide is used as a lyophilization aid that is added just prior to lyophilization and where the structure of C-m-HDLs is already formed. This is supported by the fact that the use of a disaccharide during synthesis of the C-m-HDLs described herein provides for a smaller average diameter as compared to C-m-HDLs made without the use of a disaccharide as described herein. Moreover, the smaller sized C-m-HDL particles are retained during lyophilization and after reconstitution of the lyophilized composition as compared to the average size of reconstituted lyophilize C-m-HDLs made using a disaccharide solely as a lyophilization aid. This demonstrates that the addition of the disaccharide during synthesis of these C-m-HDLs is not equivalent to the addition of the disaccharide to a solution of already formed n C-m-HDLs wherein the disaccharide is added as a lyophilization aid.

In addition to reducing the diameter of the C-m-HDLs described herein, the present disclosure provides multiple other advantages, which can be achieved by using a disaccharide during the synthesis of the micellar nanoparticles.

In some embodiments, the use of a disaccharide, pursuant to the methods described herein, can result in the following: the resulting population of C-m-HDLs provides for a tighter distribution for their diameter distribution than C-m-HDLs made without the disaccharide; the diameter of the resulting population of C-m-HDLs remains substantially the same both before and after lyophilization; the resulting population of C-m-HDLs has a lower polydistribution index (PDI) as compared to C-m-HDLs made without a disaccharide; the amount of the disaccharide used to reduce the average diameter of the resulting C-m-HDLs is at least about 2 and no more than about 6 weight percent (as defined below), or more preferably at least about 3 and no more than about 6 weight percent, in the aqueous solution used to prepare such particles as compared to the diameter obtained when synthesizing in the absence of the disaccharide; aqueous solutions of the resulting C-m-HDLs are capable of lyophilization and reconstitution without material changes in the C-m-HDLs; and aqueous solutions containing about 2 to about 6 weight percent of a disaccharide will retain that disaccharide in the lyophilized product (cake or powder) which stabilizes the lyophilizate. Accordingly, in some embodiments, lyophilized C-m-HDLs comprising a disaccharide is more stable, e.g., when formulated as a reconstituted solution, relative to a lyophilized nanoparticle that does not comprise the disaccharide.

In some embodiments, there is provided a population of C-m-HDLs which population comprises:
a) an amphiphilic, alpha-helical peptide that comprises an amino acid sequence of any one or more of SEQ ID NO: 1 through SEQ ID NO:36;
b) one or both of sphingomyelin and phosphatidyl choline, and optionally one or more additional lipids or phospholipids; and
c) a conjugate comprising an anchor moiety molecule and a chemotherapeutic agent that are releasably attached to each other through a cleavable bond;
wherein said C-m-HDLs in the population further comprise:
a hydrophilic exterior surface and a hydrophobic core; and
an average particle diameter of about 12 nanometers to about 13.5 nanometers as measured by dynamic light scattering with at least about 70% of said particles being within plus/minus about 3 nanometers of the average particle diameter of about 12 to about 13.5 nanometers (i.e., about 9 to about 16.5 nanometers).

In some embodiments, this disclosure describes a population of C-m-HDLs comprising:
(a) an amphiphilic, alpha-helical peptide that comprises an amino acid sequence of any one of SEQ ID NO:25, SEQ ID NO:28, SEQ ID NO:34, SEQ ID NO:35, SEQ ID NO: 36, or combinations thereof;
(b) one or both of sphingomyelin and phosphatidyl choline, and optionally one or more additional lipids or phospholipids; and
(c) a conjugate comprising an anchor moiety molecule and paclitaxel that are releasably attached to each other through a cleavable bond; and
wherein the C-m-HDLs in the population further comprise:
   a hydrophilic exterior surface and a hydrophobic core; and
   an average particle diameter of from about 12 to about 13.5 nanometers, as measured by dynamic light scattering, with at least about 70% of said particles being within plus/minus about 3 nanometers of the average particle diameter of about 12 to about 13.5 nanometers (i.e., about 9 to about 16.5 nanometers).

In some embodiments, the disaccharide is selected from sucrose, lactose, maltose, trehalose, cellobiose and lactulose.

Likewise, it is contemplated that simple sugars such as glucose, glucosamine, fructose, galactose, and the like could be employed instead of a disaccharide. Likewise, trisaccharides and tetrasaccharides can also be considered for such use.

In some embodiments, the disaccharide comprises any two naturally occurring sugars in an alpha acetal oxygen linkage formation.

In some embodiments, the cleavable (releasable) bond is selected from an ester, a thioester, a carbonate, a thiocarbonate, a carbamate, or a thiocarbamate bond.

As noted above, without being limited to any theory, since the addition of a disaccharide occurs during the synthesis of the nanoparticles, the disaccharide is integrated into the methods for making the nanoparticle. This would be in contrast to use of a disaccharide as a lyophilization aid where it is added just prior to lyophilization. Our belief is that the average diameter of the C-m-HDLs is reduced in the first instance whereas there is little to no change in the average diameter of the C-m-HDLs when the disaccharide is used as a lyophilization aid. This demonstrates that the addition of the disaccharide during synthesis of these C-m-HDLs is not equivalent to the addition of the disaccharide to a solution of already formed nanoparticles as a lyophilization aid.

In some embodiments, an anchor moiety is cholesterol or a tocotrienol the latter of which includes the β-, γ-, and δ-tocotrienol isoforms. In a preferred embodiment the anchor moiety is cholesterol or δ-tocotrienol.

Table 1 above provides mertansine-δ-tocotrienol conjugates with a thiocarbonate linker. It is understood that the δ-tocotrienol portion of the conjugate can be replaced by cholesterol, coprostanol, campesterol, brassicasterol, sitosterol, stigmasterol, β-tocotrienol, γ-tocotrienol, β-tocopherol, γ-tocopherol, δ-tocopherol, ceramide, and diacylglycerol where ceramide (n is from zero to ten) which has the formula:

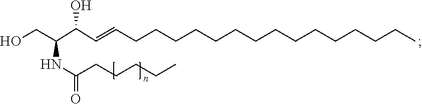

and diacylglycerol (n1 and n2 are independently 0 to 4) is represented by the formula:

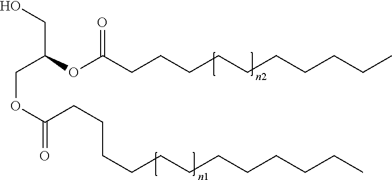

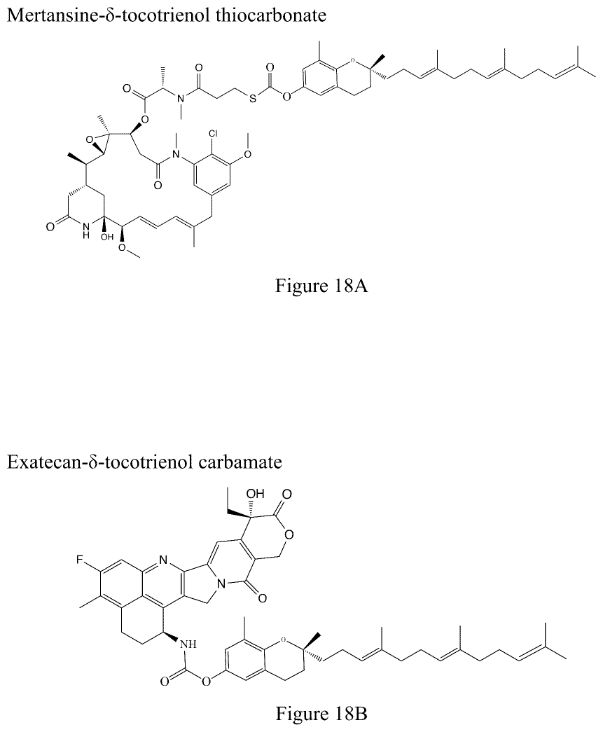

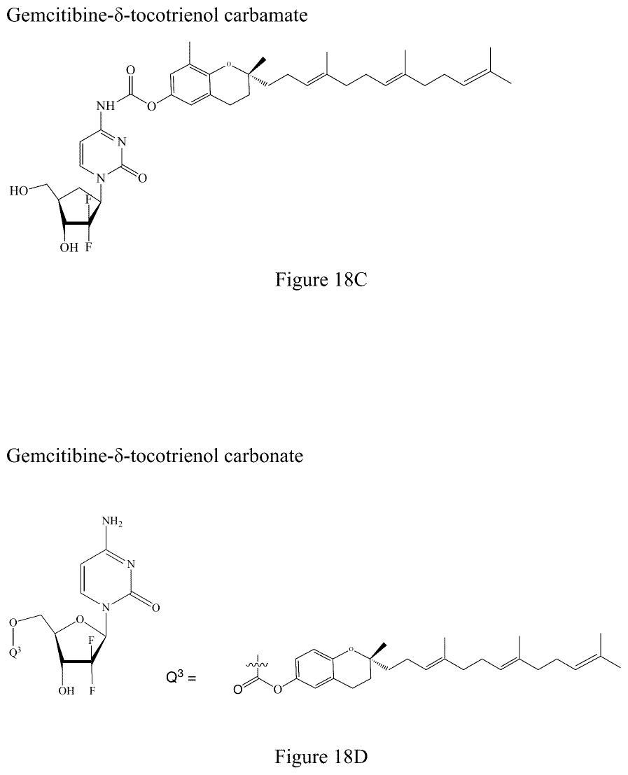

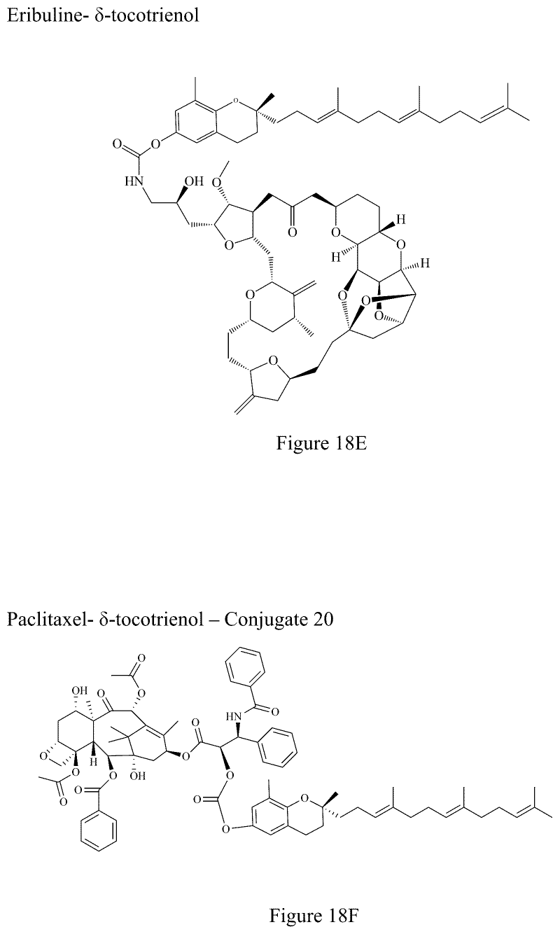

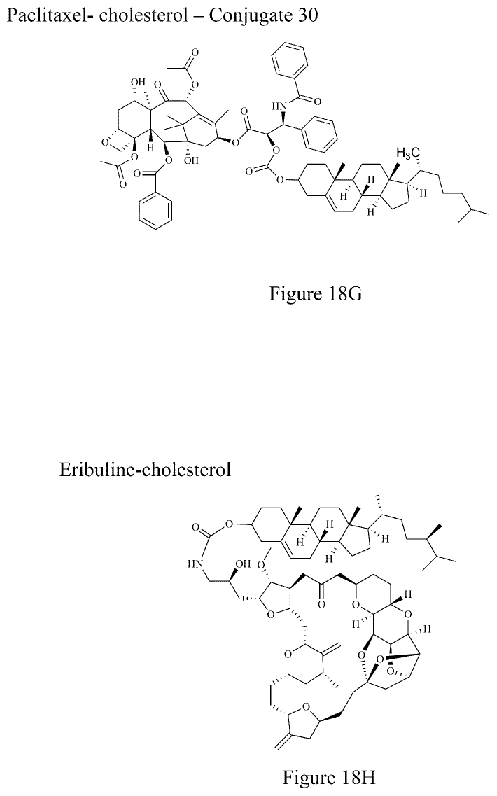

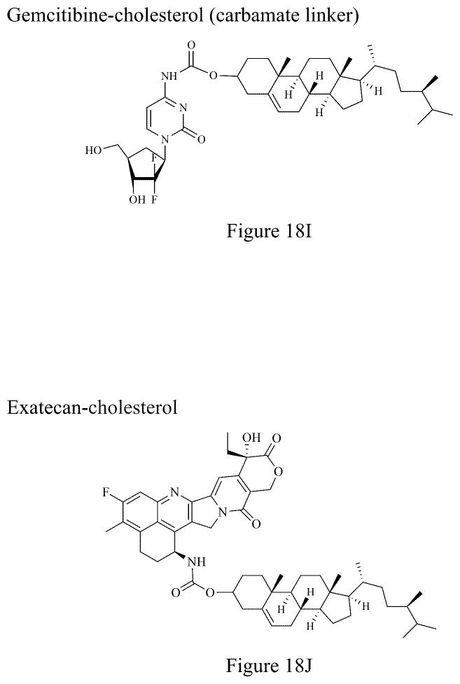

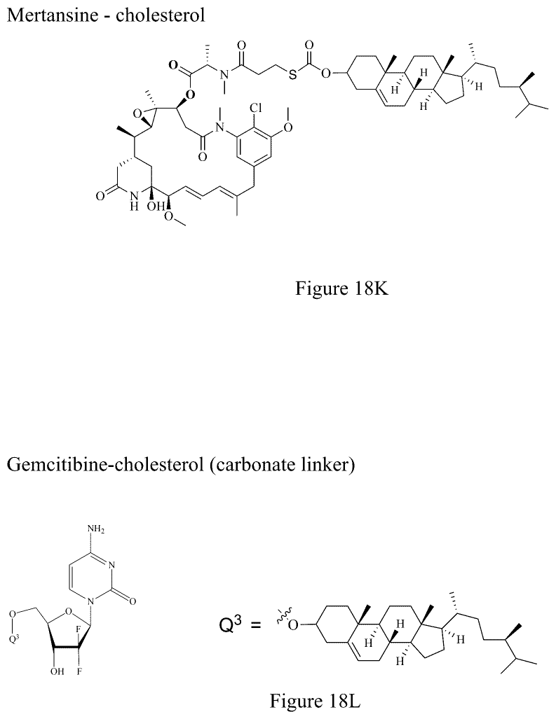

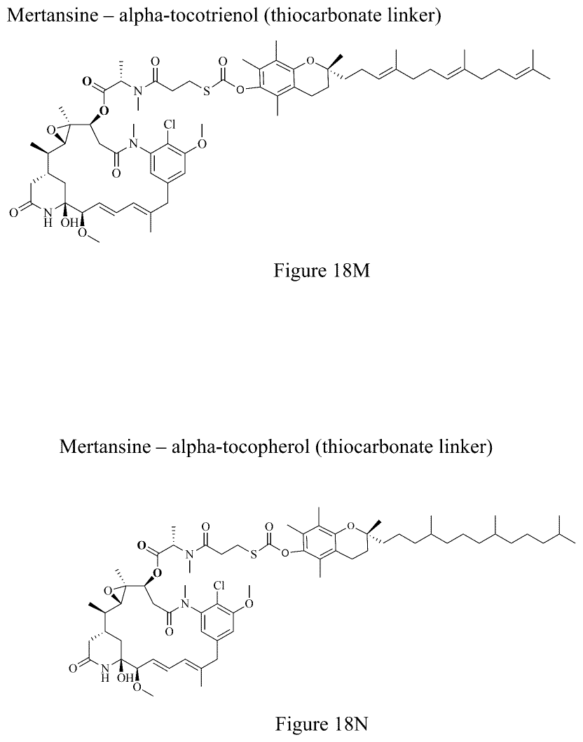

FIGS. 18A-18N provide a set of preferred conjugates for use in generating the C-m-HDLs described herein. For each of these structures, the δ-tocotrienol anchor moiety of the conjugate can be replaced by a different anchor such as cholesterol, coprostanol, campesterol, brassicasterol, sitosterol, stigmasterol, β-tocotrienol, γ-tocotrienol, β-tocopherol, γ-tocopherol, δ-tocopherol, ceramide, or diacylglycerol provided that the anchor replacement is capable of forming a cleavable covalent bond with the chemotherapeutic agent and is also absorbable into a tumor cell by SR-BI. In addition, the chemotherapeutic agent can be replaced by another chemotherapeutic agent provided that such other agents contain a reactive hydroxyl, carboxyl, amino, or thiol group capable of forming a cleavable bond linking the chemotherapeutic agent to the anchor compound.

In some embodiments, there is provided a method for preparing a solution of a fully dissolved amphiphilic, alpha-helical peptide selected from an amino acid sequence as provided by SEQ ID NO:25, SEQ ID NO:28, SEQ ID NO:34, SEQ ID NO:35, SEQ ID NO: 36, and combinations thereof in an aqueous-ethanol co-solvent, which method comprises:
a) combining in any order from about 50 to about 90 weight percent ethanol and from about 5 to about 50 weight percent water and about 0 to about 7.5 weight percent benzyl alcohol as needed depending on the character of the chemotherapeutic agent;
b) adding from about 2 to about 10 weight percent of an acid based on the weight of a) above;
c) adding from about 1 to about 2.5 weight percent of an amphiphilic, alpha-helical peptide selected from an amino acid sequence as provided by SEQ ID NO:25, SEQ ID NO:28, SEQ ID NO:34, SEQ ID NO:35 and SEQ NO: 36 based on the total weight of the solvent solution of b) above; and stirring until a clear solution is obtained.

In some embodiments, there is provided a method for preparing a composition suitable for use in making C-m-HDLs as described herein which method comprises: preparing a composition designated as “Composition 1”:
a) by combining in any order:
   i) ethanol and water to form a cosolvent having from about 50 to about 90 weight percent ethanol and from about 5 to about 50 weight percent water and about 0 to about 7.5 weight percent benzyl alcohol as needed depending on the character of the chemotherapeutic agent;
   ii) about 2 to 10 weight percent of an acid based on the weight of the cosolvent; and
   iii) about 1 to 2.5 weight percent of a peptide of SEQ ID NO. 25, SEQ ID NO: 28, SEQ ID NO: 34, SEQ ID NO: 35, or SEQ ID NO: 36 based on the weight of the cosolvent of ii);
      wherein the total amount of i), ii) and iii) equals 100 weight percent, and stirring the composition until the solution is clear evidencing that the peptide is completely dissolved;
b) combining the solution generated in a) with from about 3 to about 6 weight percent of a lipid composition based on the weight of the solution of b) wherein said lipid composition comprises:
   iv) about 50 to 70 weight percent of an additional lipid component such as 1-palmitoyl-2-oleoyl-phosphatidylcholine (POPC) (commercially available from Lipoid, Newark, New Jersey, USA)
   v) about 18 to about 30 weight percent of sphingomyelin (SM) (commercially available from Lipoid, Newark, New Jersey, USA), and
   vi) about 12 to about 35 weight percent of a conjugate as described above;
   and further wherein the combined solution generated in a) and the solution of b) equals 100 weight %; and
c) stirring as necessary to provide for a clear solution and then filter, wherein the clear filtered solution is “Composition 1.”

In some embodiments, there is provided a method for reducing the average diameter of a population of C-m-HDLs which method comprises:
a) combining water with from about 2 to about 6 weight percent, or more preferably about 3 to about 6 weight percent of a disaccharide and mixing until homogeneous, wherein the mix is designated as “Composition 2”;
b) adding Composition 2 to a first loading chamber of a three-chamber mixing device which comprises two separate loading chambers and a single reaction chamber;
c) adding an appropriate amount of Composition 1 prepared as above into the other loading chamber of said two loading chambers in the device; and
d) chaotically mixing the two compositions into the reaction chamber under a controlled flow rate ratio of from about 4:1 to about 10:1 of Composition 2 to Composition 1 at a temperature of from about 30° to about 70° C. while maintaining the flow from each chamber until mixing is complete while maintaining a temperature of from about 30° to about 70° C.;
thereby providing a nanoparticle suspension having an average diameter for the nanoparticles of about 11.5 nanometers to about 14 nanometers, and more preferably about 12 nanometers to about 13 nanometers.

In some embodiments, the controlled flow rate ratio is about 7:1 of Composition 2 to Composition 1.

In some embodiments, the flow rate of Composition 2 into the chaotically mixing reaction chamber is about 3.5 mL/min and the flowrate of Composition 1 into the chaotically mixing reaction chamber is about 0.5 mL/min.

In some embodiments, the method described above further comprises removing at least a portion of the ethanol and acetic acid by either dialysis or tangential flow filtration (TFF).

In some embodiments, the resulting suspension is sterile filtered preferably using a 0.2 micron filter.

In some embodiments, the method for reducing the average diameter of a population of C-m-HDLs further comprises lyophilizing the composition. Such lyophilization does not materially alter the physical dimensions of the C-m-HDLs.

In some embodiments, the amount of disaccharide in the lyophilized composition ranges from about 10 weight percent to about 500 weight percent disaccharide based on the weight of the nanoparticles.

In some embodiments, the amount of disaccharide in the final composition ranges from about 20 weight percent to about 200 weight percent disaccharide based on the weight of the nanoparticles.

In some embodiments, the disaccharide is selected from sucrose, lactose, maltose, trehalose, cellobiose and lactulose.

In some embodiments, the disaccharide is sucrose.

In some embodiments, the average diameter size of the C-m-HDLs after synthesis is reduced by at least about 15%, or by about 20%, or by about 25% by the presence of from about 2 weight percent to about 6 weight percent disaccharide in Composition 2 described above as compared to the average diameter of the C-m-HDLs s after synthesis in the absence of a disaccharide. The average diameter of the C-m-HDLs described herein corresponds substantially after synthesis but prior to lyophilization as compared to after synthesis and after lyophilization evidencing that lyophilization in the presence of a disaccharide causes no material change is particle size.

### Synthesis

The anchor moieties described herein are lipidic compounds that are transported into cells by SR-BI. Such anchor moieties are coupled to a chemotherapeutic agent by a cleavable bond and when so coupled are referred herein as “conjugates.” Such conjugates can be prepared from readily available starting materials using the methods described herein.

It will be appreciated that where typical process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and P. G. M. Wuts, Protecting Groups in Organic Synthesis, Third Edition, Wiley, New York, 1999, and references cited therein.

The starting materials for the following reactions are generally known compounds or can be prepared by known procedures or obvious modifications thereof. For example, many of the starting materials are available from commercial suppliers such as SigmaAldrich (St. Louis, Missouri, USA), Bachem (Torrance, California, USA), Emka-Chemce (St. Louis, Missouri, USA). Others may be prepared by procedures, or obvious modifications thereof, described in standard reference texts such as Fieser and Fieser's Reagents for Organic Synthesis, Volumes 1-15 (John Wiley, and Sons, 2016), Rodd's Chemistry of Carbon Compounds, Volumes 1-5, and Supplementals (Elsevier Science Publishers, 2001), Organic Reactions, Volumes 1-40 (John Wiley, and Sons, 2019), March's Advanced Organic Chemistry, (John Wiley, and Sons, 8th Edition, 2019), and Larock's Comprehensive Organic Transformations (VCH Publishers Inc., 1989). The disclosures of which are incorporated herein by reference in their entirety.

### Peptides

Peptides as used herein refer to peptides that are suitable for use in the micellar nanoparticles, compositions, and/or methods this disclosure. Illustrative peptides are provided in the Table 7 below.

TABLE 7

| SEQ ID | | |
|---|---|---|
| NO | Sequence | Notes |
| 1 | DVXQXXKEXXXQXXEKXKQV | Position 3 is L or F; position 5 is A or Aib; |
| | | position 6 is L or F; position 9 is A, L, F, or Aib; |
| | | position 10 is L or F; position 11 is A, Aib, or N; |
| | | position 13 is L or F; position 14 is A, L, F, or |
| | | Aib; position 17 is W, F. or L |
| 2 | DVFQALKELFAQLLEKWKQV | NA |
| 3 | DVFQXLKELFNQLLEKWKQV | Position 5 is Aib |
| 4 | DVFQXLKELLAQLLEKFKQV | Position 5 is Aib |
| 5 | DVFQXLKELLNQLLEKFKQV | Position 5 is Aib |
| 6 | DVFQXLKELLNQLXEKFKQV | Position 5 is Aib; position 14 is Aib |
| 7 | DVFQXLKELLNQLXEKWKQV | Position 5 is Aib; position 14 is Aib |
| 8 | DVFQALKELLAQLLEKFKQV | NA |
| 9 | DVFQALKELLNQLLEKFKQV | NA |
| 10 | DVFQXLKELFAQLLEKWKQV | Position 5 is Aib |
| 11 | DVFQXLKELFNQLLEKWKQV | Position 5 is Aib |
| 12 | DVFQXLKELFNQLLEKFKQV | Position 5 is Aib |
| 13 | DVFQALKELFAQLXEKWKQV | Position 14 is Aib |
| 14 | DVFQALKELFNQLXEKWKQV | Position 14 is Aib |
| 15 | DVFQALKELFNQLXEKFKQV | Position 14 is Aib |
| 16 | DVFQAFKEAFAQLFEKWKQV | NA |
| 17 | DVFQAFKEXFAQLFEKWKQV | Position 9 is Aib |
| 18 | DVFQXFKEXFAQLFEKWKQV | Position 5 is Aib; position 9 is Aib |
| 19 | DVFQAFKEAFXQLFEKWKQV | Position 11 is Aib |
| 20 | DVFQAFKEXFXQLFEKWKQV | Position 9 is Aib; position 11 is Aib |
| 21 | DVFQXFKEXFXQLFEKWKQV | Position 5 is Aib; position 9 is Aib; |
| | | position 11 is Aib |
| 22 | DVFQALKELFNQLLEKWKQV | NA |
| 23 | DVFQXLKELLNQLLEKLKQV | Position 5 is Aib |
| 24 | XXXXXXXXXXXXXXXXXXXX | Position 1 is D or E; |
| | | position 2 is V, I, or L; |
| | | position 3 is L, I, V, W, Y, Aib, Amv, or F; |
| | | position 4 is Q or N; |
| | | position 5 is K, R, H, Orn; |
| | | position 6 is L, I, V, W, Y, Aib, Amv, or F; |
| | | position 7 is A, G, S, V, Aib, or Amv; |
| | | position 8 is E or D; |
| | | position 9 is A, G, S, L, F, V, Amv, or Aib; |
| | | position 10 is L, I, V, W, Y, Aib, Amv, or F; |
| | | position 11 is A, G, S, Aib, Amv, V, or N; |
| | | position 12 is Q or N; |
| | | position 13 is L, I, V, W, Y, Aib, Amv, or F; |
| | | position 14 is A, G, S, L, F, V, Amv, or Aib; |
| | | position 15 is E or D; |
| | | position 16 is K, R, H, Orn; |
| | | position 17 is W, F, Y, I, V, or L; |
| | | position 18 is K, R, H, Orn; |
| | | position 19 is Q or N; |
| | | position 20 is V, I, or L |
| 25 | DVFQKLXELFNQLLEKWKQV | Position 7 is Aib |
| 26 | DVFQKLVELFNQLLEKWKQV | NA |
| 27 | DVXQKLFELFNQLLEKWKQV | Position 3 is Aib |
| 28 | DVFQKLXELFNQLLEKFKQV | Position 7 is Aib |
| 29 | DVFQKLVELFNQLLEKFKQV | NA |
| 30 | DVXQKLFELFNQLLEKFKQV | Position 3 is Aib |
| 31 | DVLQKFXELFNQLLEKWKQV | Position 7 is Aib |
| 32 | DVXQKFLELFNQLLEKWKQV | Position 3 is Aib |
| 33 | DVFQKLLEXFNQLLEKWKQV | Position 9 is Aib |
| 34 | DVFQKLXELFNQXLEKWKQV | Position 7 is Aib; position 13 is Aib |
| 35 | DVFQKLXELFNQLXEKWKQV | Position 7 is Aib; position 14 is Aib |
| 36 | DXFQKLXELFNQLXEKWKQV | X in each instance is Aib |

Any of the foregoing peptides can be used in preparing a population of C-m-HDLs as disclosed herein. See, e.g., FIG. 9. Any of the foregoing peptides described in Table 7, or disclosed herein, i.e., any one of the peptides having an amino acid sequence as set forth in any one of SEQ ID NOs: 1-36, can be used in preparing C-m-HDLs as described herein.

In some embodiments, a peptide of the present disclosure comprises an amino acid sequence that is at least 80% identical, at least 85% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence set forth in any one of SEQ ID NOs: 1-36.

In some embodiments, a peptide of the present disclosure comprises an amino acid sequence that is at least 80% identical, at least 85% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence:

(SEQ ID NO: 25)
DVFQKL{AIB}ELFNQLLEKWKQV.

In some embodiments, a peptide of the present disclosure comprises an amino acid sequence that is at least 80% identical, at least 85% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence:

(SEQ ID NO: 28)
DVFQKL{AIB}ELFNQLLEKFKQV.

In some embodiments, a peptide of the present disclosure comprises an amino acid sequence that is at least 80% identical, at least 85% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence:

(SEQ ID NO: 34)
DVFQKL{AIB}ELFNQ{AIB}LEKWKQV.

In some embodiments, a peptide of the present disclosure comprises an amino acid sequence that is at least 80% identical, at least 85% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence:

(SEQ ID NO: 35)
DVFQKL{AIB}ELFNQL{AIB}EKWKQV.

In some embodiments, a peptide of the present disclosure comprises an amino acid sequence that is at least 80% identical, at least 85% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence:

(SEQ ID NO: 36)
D{Aib}FQKL{Aib}ELFNQL{Aib}EKWKQV.

In some embodiments, a peptide of the present disclosure consists essentially of an amino acid sequence of:

(SEQ ID NO: 25)
DVFQKL{AIB}ELFNQLLEKWKQV.

In some embodiments, a peptide of the present disclosure consists essentially of

(SEQ ID NO: 28)
DVFQKL{AIB}ELFNQLLEKFKQV.

In some embodiments, a peptide of the present disclosure consists essentially of an amino acid sequence of:

(SEQ ID NO: 34)
DVFQKL{AIB}ELFNQ{AIB}LEKWKQV.

In some embodiments, a peptide of the present disclosure consists essentially of an amino acid sequence of:

(SEQ ID NO: 35)
DVFQKL{AIB}ELFNQL{AIB}EKWKQV.

In some embodiments, a peptide of the present disclosure consists of an amino acid sequence of:

(SEQ ID NO: 36)
D{Aib}FQKL{Aib}ELFNQL{Aib}EKWKQV.

In some embodiments, a peptide of the present disclosure consists of:

(SEQ ID NO: 25)
DVFQKL{AIB}ELFNQLLEKWKQV.

In some embodiments, a peptide of the present disclosure consists of:

(SEQ ID NO: 28)
DVFQKL{AIB}ELFNQLLEKFKQV.

In some embodiments, a peptide of the present disclosure consists of:

(SEQ ID NO: 34)
DVFQKL{AIB}ELFNQ{AIB}LEKWKQV.

In some embodiments, a peptide of the present disclosure consists of:

(SEQ ID NO: 35)
DVFQKL{AIB}ELFNQL{AIB}EKWKQV.

In some embodiments, a peptide of the present disclosure consists of:

SEQ ID NO: 36)
D{Aib}FQKL{Aib}ELFNQL{Aib}EKWKQV.

Methods for the synthesis of these peptides are well known in the art. For the sake of completion, Table 8 provides a complete list of 1 letter and 3 letter abbreviations for the naturally occurring amino acids:

TABLE 8

| Amino acid | 3-letter abbreviation | 1-letter abbreviation |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Ornithine | Orn | |
| 2-aminoisobutyric acid | Aib | |
| Alpha-amino-3-methylvaleric | Amv | |
| acid | | |

### Lipids

The C-m-HDLs described herein comprise multiple copies of one or more peptides as provided herein that self-assembled with one or more lipid components and a conjugate into the C-m-HDLs. In some embodiments, the lipid component comprises one or both of sphingomyelin and phosphatidyl choline, and optionally one or more additional lipids or phospholipids. The conjugate comprises a chemotherapeutic agent covalently bound to an anchor moiety by a cleavable bond wherein the anchor moiety is absorbable by SR-BI (such as those provided in Tables 1-6). These C-m-HDLs provide for targeted tumor binding to SR-BI on tumor cells that overexpress this receptor. Upon binding, the conjugate is delivered intracellularly which then allows for cleaving of the cleavable bond by intracellular enzymes. Upon cleavage, free chemotherapeutic is generated in the cell.

In some embodiments, the lipid component comprises or consists essentially of one or both of sphingomyelin and phosphatidyl choline, and optionally one or more additional lipids or phospholipids. In some embodiments, the C-m-HDLs comprise multiple copies of a peptide as described herein and a lipid component wherein the lipid component comprises sphingomyelin and one or more additional phospholipids where the additional phospholipid is selected from the group consisting of phosphatidylcholine, polyethylene glycol-phosphatidylethanolamine (PEG-PE), phosphatidylethanolamine, phosphatidylglycerol, phosphatidylserine, phophatidylinositol, cardiolipin, and any combination thereof.

In some embodiments, the C-m-HDLs described herein comprise multiple copies of a peptide as described herein and a lipid component which comprises one or both of sphingomyelin and phosphatidylcholine. In some embodiments, C-m-HDLs as described herein comprise multiple copies of a peptide as described herein and a lipid component which comprises sphingomyelin and 1-palmitoyl-2-oleoyl phosphatidylcholine (POPC). In some embodiments, the C-m-HDLs described herein comprise multiple copies of a peptide as described herein and a lipid component which comprises sphingomyelin and phosphatidylethanolamine. In some embodiments, the C-m-HDLs described herein comprise multiple copies of a peptide as described herein and a lipid component which comprises sphingomyelin and poly-(ethyleneglycol) phosphatidylethanolamine. In some embodiments, the C-m-HDLs describe herein comprise multiple copies of a peptide as described herein and a lipid component which comprises sphingomyelin and phosphatidylserine. In some embodiments, the C-m-HDLs describe herein comprise a peptide described above and a lipid component which comprises sphingomyelin and cardiolipin.

In some embodiments, the C-m-HDLs described herein comprises multiple copies of a peptide as described herein and the lipid component that consists essentially of sphingomyelin and one or more additional phospholipids where the one or more additional phospholipid is selected from phosphatidylcholine, polyethylene glycol phosphatidylethanolamine (PEG-PE), phosphatidylethanolamine, phosphatidylglycerol, phosphatidylserine, phosphatidylinositol, cardiolipin, and any combination thereof.

In some embodiments, the C-m-HDLs described herein consists essentially of multiple copies of a peptide as described herein and the lipid component consists essentially of sphingomyelin and phosphatidylcholine. In another embodiment, the nanoparticles described herein consists essentially of a peptide as described above and the lipid component cons lipid component consists essentially of sphingomyelin and 1-palmitoy 1-2-oleoy 1-phosphatidylcholine (POPC).

In some embodiments, the C-m-HDLs described herein comprise multiple copies of a peptide as described herein and the lipid component consists essentially of sphingomyelin and one or more additional phospholipid(s) where the one or more additional phospholipid(s) is (are) selected from the group consisting of phosphatidylcholine, polyethylene glycol-phosphatidylethanolamine (PEG-PE), phosphatidylethanolamine, phosphatidylglycerol, phosphatidylserine, phosphatidylinositol, cardiolipin, and any combination thereof where the molar ratio of phospholipid to sphingomyelin is from about 95:5 to about 10:90. In another embodiment the molar ratio of phospholipid to sphingomyelin is from about 90:10 to about 20:80. In still another embodiment the molar ratio of phospholipid to sphingomyelin is from about 25:75 to about 35:65. In another embodiment the molar ratio of phospholipid to sphingomyelin is about 30:70. In another embodiment, the molar ratio of phospholipid to sphingomyelin is from about 80:20 to about 60:40. In yet another embodiment the molar ratio of phospholipid to sphingomyelin is from about 75:25 to about 65:35. In still another embodiment the molar ratio of phospholipid to sphingomyelin is about 70:30.

The fatty acid constituents of the phospholipids include fatty acids according to the formula: R—COOH, wherein R is an alkyl group having from about 10 to about 30 carbon atoms or an alkenyl group having from about 10 to about 30 carbon atoms and having from one to six double bonds which are preferably in the “cis” configuration. Examples of suitable fatty acids include, but are not limited to, phytanic acid, linolenic acid, linoleic acid, docosatetraenoic acid, oleic acid, caprylic acid, lauric acid, arachidonic acid, docosahexaneic acid, myristic acid and palmitic acid. The pair of fatty acids esterified to the glycerol backbone of a particular phospholipid may be identical or each may be a different type of fatty acid.

In some embodiments, conjugates described herein are synthesized as shown in Scheme 1 below.

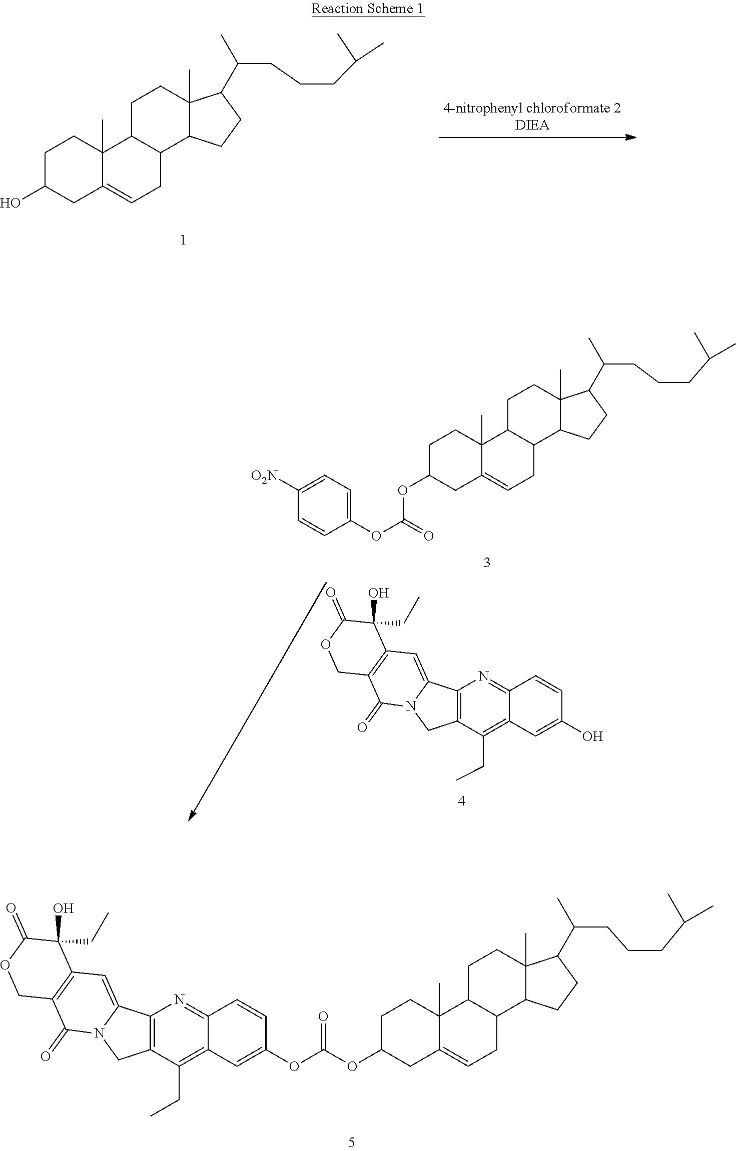 [SMILES: CC(C)CCCC(C)C1CCC2C3CC=C4CC(O)CCC4(C)C3CCC12C ; CC(C)CCCC(C)C1CCC2C3CC=C4CC(OC(=O)Oc5ccc([N+](=O)[O-])cc5)CCC4(C)C3CCC12C ; CCc1c2c(nc3ccc(O)cc13)-c1cc3c(c(=O)n1C2)COC(=O)[C@]3(O)CC ; CCc1c2c(nc3ccc(OC(=O)OC4CCC5(C)C(=CCC6C5CCC5(C)C(C(C)CCCC(C)C)CCC65)C4)cc13)-c1cc3c(c(=O)n1C2)COC(=O)[C@]3(O)CC]

In Reaction Scheme 1, approximately equimolar amounts of cholesterol, 1, was used in combination with 4-nitrophenyl chloroformate, 2, in a suitable inert solvent such as chloroform, toluene, tetrahydrofuran, ethyl acetate, N,N-dimethylformamide, and the like. The reaction solution is maintained at a temperature of from about 0° to about 20° C. in the presence of any of a number of suitable bases such as trimethylamine, pyridine, DIEA (diisopropylethyl amine), and the like. The base is present to scavenge the acid generated by the reaction. The reaction is maintained with gentle stirring until the reaction is substantially complete as evidenced by, e.g., thin layer chromatography. At that time, the reaction is then stopped, and the product recovered by conventional means and optionally purified by, for example, high-performance liquid chromatography (HPLC), crystallization, and the like to provide the resulting 4-nitrophenyl carbonate, compound 3. Alternatively, the reaction product can be used in the next step without purification and/or isolation.

Next, approximately equimolar amounts of compound 3 are combined with 7-ethyl-hydroxy camptothesin, compound 4, in a suitable inert solvent such as chloroform, toluene, tetrahydrofuran, ethyl acetate, N,N-dimethylformamide, and the like maintained at a temperature of from 0° to about 20° C. under conditions sufficient to for the phenolic hydroxyl group of compound 4 to displace the 4-nitrophenoxy group. Such reactions are well known in the art. In some cases, conversion of the hydroxyl functionality on the chemotherapeutic agent to a mesyl or tosyl group will facilitate the reaction. Recovery of the resulting conjugate, compound 5, proceeds as above.

In general, the use of similar chemistry with chemotherapeutic agents containing a reactive amino group will provide for carbamate linkages; whereas chemotherapeutic agents comprising a carboxyl group will provide for esters; and chemotherapeutic agents containing a thiol group will provide for thioesters or thiocarbamates. Further exemplification is provided the examples below.

As is well known in the art, the use of protecting groups may be necessary when the chemotherapeutic compound comprises multiple binding functionalities. Restricting reaction to a specific functional group will requiring the use of blocking groups well known in the industry.

### C-m-HDL Formation

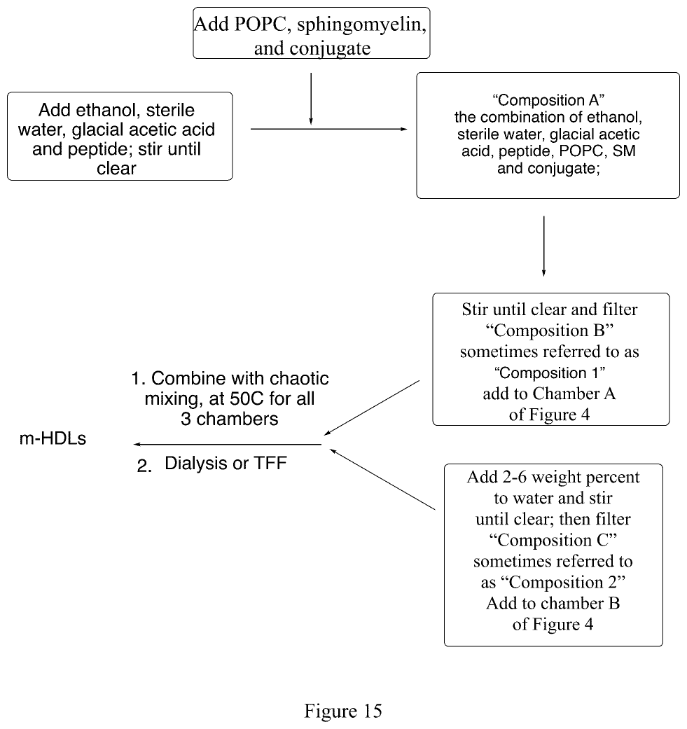

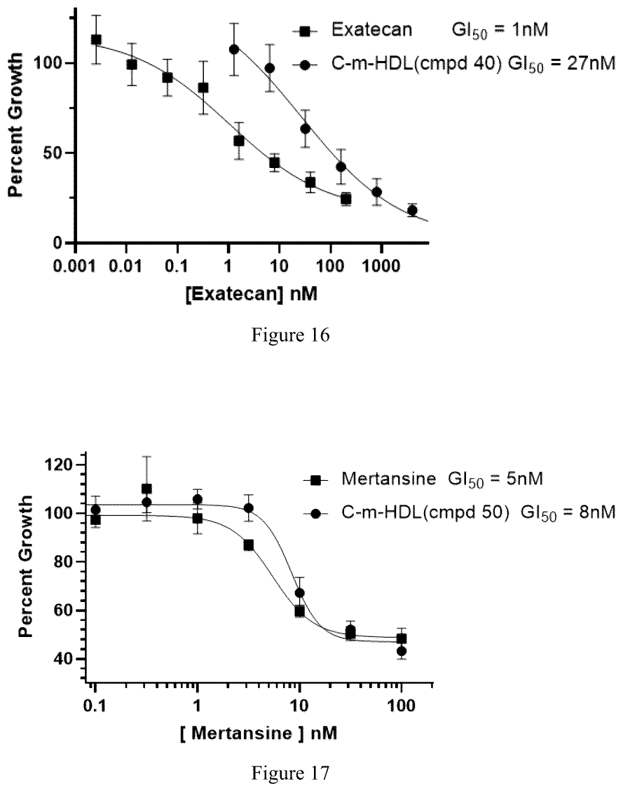

The components described above are then combined as shown below to provide for the C-m-HDLs. These C-m-HDLs are prepared by a multi-step process that is illustrated in FIG. 15. Specifically, a peptide as described above (e.g., SEQ ID NO: 35) is added into a mixed solvent system comprising ethanol, water and an organic or inorganic acid. The acid is utilized to provide for complete solubilization of the peptide into the solution.

FIG. 15 provides for a non-limiting general procedure for preparing the C-m-HDLs. It is understood that other equivalent methods for mixing and lyophilization can be used.

### Methodology

A method for reducing the size of C-m-HDLs which method comprises:
a) combining the following components into a first container:
   i) an ethanol and water cosolvent having from about 50 to about 90 weight percent ethanol and about 5 to about 50 weight percent water and about 0 to about 7.5 weight percent benzyl alcohol as needed depending on the character of the chemotherapeutic agent;
   ii) about 2 to about 10 weight percent of an acid based on the weight of the cosolvent;
   iii) about 1 to about 2.5 weight percent of a peptide of SEQ ID NO:35 or any of the other peptides discussed above based on the weight of the cosolvent plus the acid, wherein the total of i), ii) and iii) equals 100 weight %, and stirring the composition until the peptide is completely dissolved;
b) then combining into the composition of a) above from about 3 to about 6 weight percent based on the weight of the solution of a) above of a lipid composition comprising:
   iv) about 40 to 70 weight percent of 1-palmitoyl-2-oleoyl-phosphatidylcholine (POPC) (commercially available from Lipoid, Newark, New Jersey, USA) where optionally other lipids can be used in place of or in addition to POPC.
   v) about 30 to about 60 weight percent of sphingomyelin (SM) (commercially available from Lipoid, Newark, New Jersey, USA), and
   vi) about 12 to about 35 weight percent of a conjugate comprising a lipid conjugate (anchor moiety) and a chemotherapeutic agent such as paclitaxel both of which are connected by a cleavable covalent bond;
   wherein the total for each of iv), v) and vi) equals 100 weight percent and is referred to as Composition 2;
and further wherein the aggregate total of the composition in a) and the lipid Composition 2 in b) equals 100 weight %;
c) intimately mixing Composition 2 with turbulence the contents of sterile water containing about 2 to about 6 weight percent of a disaccharide (Composition 1) at a temperature of from about 30° to 70° C. for a period of from 0.1 to 6 hours
d) subjecting the resulting composition to dialysis or tangential flow filtration; and
e) sterile filtering the composition;
wherein a C-m-HDLs composition is produced which has an average diameter for the nanoparticles of about 11.5 to about 14 nanometers, and preferably 12 to about 13.5 nanometers, and have a distribution of particle sizes with at least about 70% or more preferably at least about 65% of said particles are within plus/minus 3 nanometers.

In some embodiments, the method above further comprises lyophilization of said sterile composition to provide for a lyophilizate.

Specific examples of preparation of C-m-HDLs are provided in the Examples set forth below.

The average diameter of the nanoparticles described in solution is assessed by dynamic light scattering conducted as described herein and is reduced from about 17 nanometers as found in the '105 patent when made in the absence of a disaccharide to about 12 to about 13.5 nanometers as described above when made in the presence of a disaccharide.

As to the addition of acid to the mixed solvent system containing the peptide, it can be an organic acid such as formic acid, citric acid, acetic acid, propanoic acid, oxalic acid, succinic acid, folic acid, benzoic acid, tartaric acid, and the like which is used neat and in an amount of from 2 to about 10 weight percent based on the weight of the cosolvent solution.

In some embodiments, the amount of organic acid employed can be used in an aqueous solution such that the amount of water employed does not alter the overall ranges of the compositions described herein.

In some embodiments, the carboxyl acid used is preferably substantially free of water (less than 1 percent) including that found as a carboxyl acid hydrate.

Regardless of the rationale, complete solubilization by altering the protonation of the peptide allows for reproducible stoichiometric amounts of each of the components found in the nanoparticles. This is because the method avoids the problem of varying amounts of insoluble peptide which alters the stoichiometric ratios in the formed nanoparticles by the inability to capture the insoluble peptide. In addition, the modified protonation of the peptide is carried over to the C-m-HDLs thereby providing for a novel C-m-HDLs having complete protonation of the free amino groups. Accordingly, in some embodiments, there is provided a C-m-HDLs as described above wherein the lysine residues of said peptide component of said C-m-HDLs are protonated at greater than 99.99 percent.

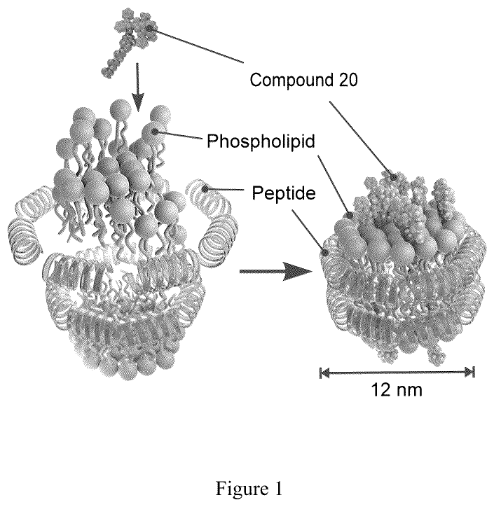

FIG. 1 provides a schematic of a postulated view of a C-m-HDL as disclosed herein. In that schematic, the amphiphilic peptide aggregates around the center of the nanoparticle extending visually around the nanoparticle. In doing so, the aggregate assembly of peptides is similar to defining a belt or strap. As depicted, the hydrophilic alpha-helical face of these peptide aggregate faces outward into the hydrophilic domain whereas the opposite surface faces inward into the hydrophobic domain. Exposed at both the top and bottom portions of the nanoparticle are the hydrophilic portions of phospholipids. These ends are from amphiphiles such as a phospholipid with the opposite hydrophobic end pointing inward and forming part of the hydrophobic core which is also formed by the conjugate wherein the anchor moiety of the conjugate (or prodrug) is selected to be essential for tumor growth whereas the chemotherapeutic is attached to the anchor moiety via a cleavable covalent bond (not shown).

Without being limited to any theory, it is proposed that the hydrophilic face of the peptide components aggregate as described above and are exposed on the surface thereby allowing for recognition by SB-R¹. Upon recognition, the transport of the anchor moiety conjugated to a chemotherapeutic agent such as paclitaxel (e.g., delta tocotrienol-paclitaxel) into the cell occurs with high efficiency. Upon intracellular absorption, intracellular enzymes hydrolyze the carbonate bond which releases the chemotherapeutic agent into the cell.

### Utility

The C-m-HDLs described herein are useful in treating disorders mediated by the overexpression of SR-BI. Such disorders include any solid mass tumors and blood borne cancers identified as having overexpression of SR-BI. As this receptor provides tumor cells with essential lipids for tumor growth, almost all or all cancers are believed to be encompassed by this definition. In some embodiments, the solid mass tumor includes, by way of example only, breast cancer (including triple negative breast cancer), bladder cancer, gastrointestinal cancers, head and neck cancers, neuroblastoma, non-small-cell lung cancer, ovarian cancer, pancreatic cancer, prostate cancer, stomach cancer, kidney cancer, liver cancer, cervical cancer, and adrenal cancer. In some embodiments, blood borne cancers include leukemia, multiple myeloma, and lymphoma. Accordingly, in some embodiments, there is provided a method for treating a patient with a disorder mediated at least in part by the overexpression of SR-BI which method comprises administering to said patient an effective amount of a composition comprising C-m-HDLs as described herein.

The treatment of such cancers encompasses the administration of the C-m-HDLs described herein to patients suffering from such a cancer in any form acceptable for treatment of that cancer. Typically, the nanoparticles are delivered intravenously as a very fine sterile suspension in a carrier such as 0.9 weight % saline or 5 weight % dextrose or in any aqueous formulation deemed suitable by the attending clinician. Other suitable forms include pulmonary delivery (e.g., aerosols), direct injection or administration into the tumor, and the like.

The amount of C-m-HDLs administered to the patient is predicated upon factors such as the age and weight of the patient, the extent that the cancer has progressed, the tumor burden, and related concerns well known to the attending clinician. In some embodiments, the amount of chemotherapeutic equivalents (e.g., paclitaxel equivalents) delivered is calculated to approximate the same amount of paclitaxel as is currently prescribed for a given patient having similar considerations. However, it is contemplated that in cases where prior use of paclitaxel was limited due to off-target toxicity, the enhanced target specificity provided by the C-m-HDLs having a number average diameter that corresponds substantially to about 12 nanometers allows for either increased amounts of paclitaxel that can be administered to the patient over the same unit of time or that the same amount of paclitaxel can be administered to the patient with fewer side effects. Such increased amounts can be 10% greater, or 20% greater, or 30% greater than the amount of paclitaxel currently administered in a single dose to a patient.

The compositions described herein employ a suitable pharmaceutically acceptable excipient typically a sterile aqueous carrier wherein the amount of the nanoparticles comprise from about 0.1 to 15 weight percent based on the weight of the carrier. U.S. Pat. No. 10,532,105 provides further information regarding suitable formulations and delivery mechanisms that are suitable for use herein.

### Benefits

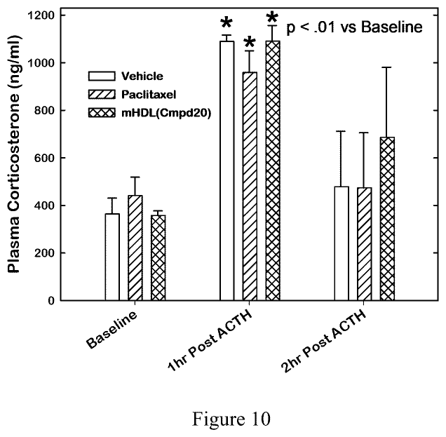

There are significant safety benefits achieved by the nanoparticles described herein including the lack of adverse liver and/or adrenal gland toxicity sufficient to remove a patient from this therapy. Both the liver and the adrenal gland are known to highly express SR-BI and might be suspected of non-target toxicity. To assess whether such expression levels would limit the use of paclitaxel containing nanoparticles described herein, an animal study was conducted to determine the compatibility of these nanoparticles for treating solid mass tumors. The animal study is reported in Example 10 and FIG. 10. Based on the results provided in that Example, there was little to no toxicity evident in the adrenal gland of the animals. The multiple dose tolerance data in Table 10 in Example 13 below indicate that, unlike a conventional paclitaxel formulation (Abraxane®), C-m-HDL (cmpd 20) avoids liver toxicity (no liver enzyme elevation in plasma).

In addition to the above, the C-m-HDLs disclosed herein provide multiple additional advantages by using a disaccharide during the synthesis including, but not limited to the following:
the resulting population of C-m-HDLs provides for average diameters with a tighter distribution than C-m-HDLs made without the disaccharide;
the diameter of the resulting population of C-m-HDLs remains substantially the same both before and after lyophilization and reconstitution;
the resulting population of C-m-HDLs has a lower polydistribution index (PDI) as compared to C-m-HDLs made without a disaccharide; for example, the PDI for C-m-HDLs prepared herein with sucrose was about 0.2 whereas those made without sucrose was about 0.4 with lower numbers showing narrower particle size distribution;
the amount of sucrose required to reduce the average diameter of the resulting C-m-HDLs must be at least about 2%, and preferably at least about 3%, and no more than about 6% of the disaccharide (as defined below) as compared to the diameter obtained when synthesizing in the absence of the disaccharide;
aqueous solutions of the resulting C-m-HDLs are capable of lyophilization and subsequent reconstitution without material changes in the C-m-HDLs including its size;
lyophilized disaccharide C-m-HDLs obviate the relative instability of aqueous solutions of such C-m-HDLs rendering aqueous solutions unacceptable for storage periods necessary between manufacture and patient administration; and
lyophilized C-m-HDLs are more stable in a reconstituted solution as compared to lyophilized nanoparticles without the disaccharide.

### EXAMPLES

The following non-limiting examples illustrate the preparation of the compositions described herein.

### Examples A and B—Synthesis of a Conjugate of Paclitaxel and Delta Tocotrienol/Cholesterol

### Example A

This conjugate comprising paclitaxel and delta tocotrienol was prepared as per Example 3 of U.S. Pat. No. 10,532,105 which has the structure:

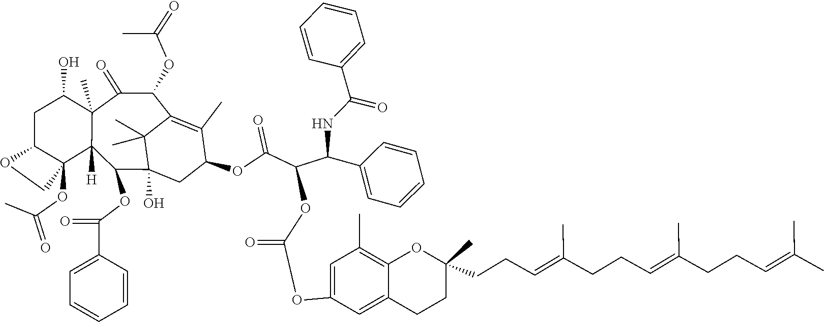 [SMILES: CC(=O)O[C@H]1C(=O)[C@@]2(C)[C@H]([C@H](OC(=O)c3ccccc3)[C@]3(O)C[C@H](OC(=O)[C@H](OC(=O)Oc4cc(C)c5c(c4)CC[C@@](C)(CC/C=C(\C)CC/C=C(\C)CCC=C(C)C)O5)[C@@H](NC(=O)c4ccccc4)c4ccccc4)C(C)=C1C3(C)C)[C@]1(OC(C)=O)CO[C@@H]1C[C@@H]2O]

Conjugate 20 (Conjugate 20 is often used interchangeably with Compound 20 or “cmpd20” as are the other conjugates disclosed below)

### Example B

Likewise, a conjugate comprising paclitaxel and cholesterol was similarly prepared and is depicted below as Conjugate 30.

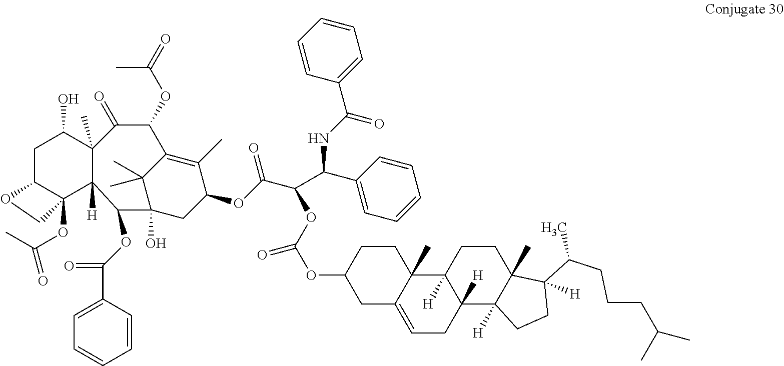

Conjugate 30 is disclosed by Stevens, et al., A Folate Receptor-Targeted Lipid Nanoparticle Formulation for a Lipophilic Paclitaxel Prodrug, Pharmaceutical Research, 21 (12): 2153-2157 (2004) which is incorporated herein by reference in its entirety.

### Example 1—Synthesis of Delta-Tocotrienol-Exatecan Conjugate with a Carbamate Linker (Conjugate 40)

The synthesis of Conjugate 40 is set forth in Reaction Scheme 2 set forth below.

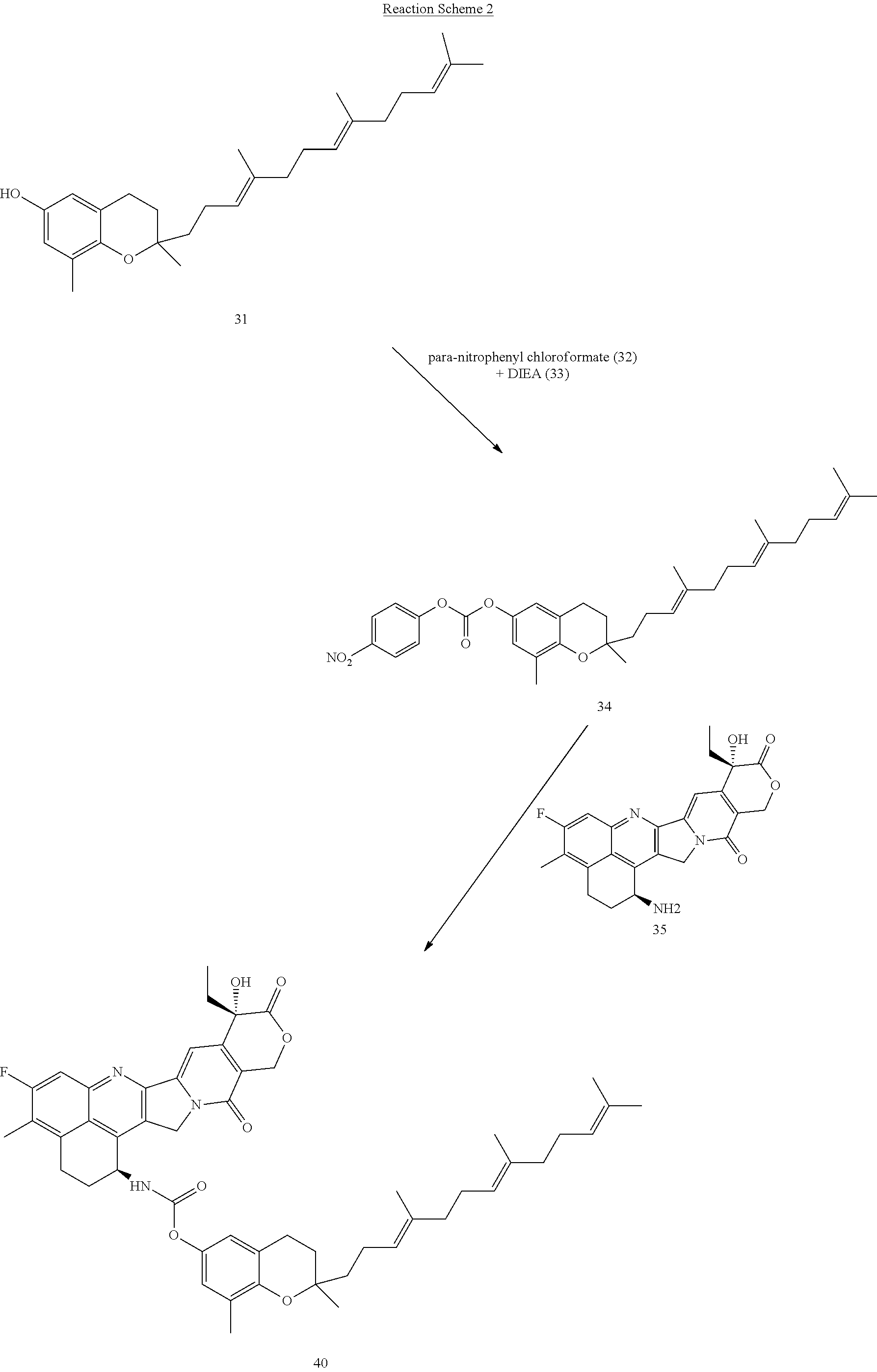

In this reaction scheme, commercially available delta-tocotrienol, compound 31, was converted to the corresponding p-nitrophenyl carbonate, compound 34, by the addition of at least about a stoichiometric equivalent of p-nitrophenylchloroformate, compound 32, in the presence of a slight excess of a base (e.g., diisopropylethylamine), compound 33, which scavenges the acid generated during the reaction. Alternatively, the reaction could be conducted in pyridine which would act both as the solvent as well as the base. The reaction conditions used were conventional and compound 34 was then converted to compound 40 by another conventional step where compound 34 was contacted with about a molar equivalent of commercially available exatecan to provide for a carbamate linkage between both groups. The reaction was conducted at room temperature and continued until substantially complete. Conjugate, 40, was recovered by chromatography to provide for a pale yellow solid.

### Example 2—Synthesis of delta-tocotrienol-mertansine conjugate with a thiocarbonate linker (Conjugate 50)

By following the procedure set forth above, the reaction proceeded above to provide for Conjugate 50 comprising delta-tocotrienol linked to mertansine via a thiocarbonate cleavable bond:

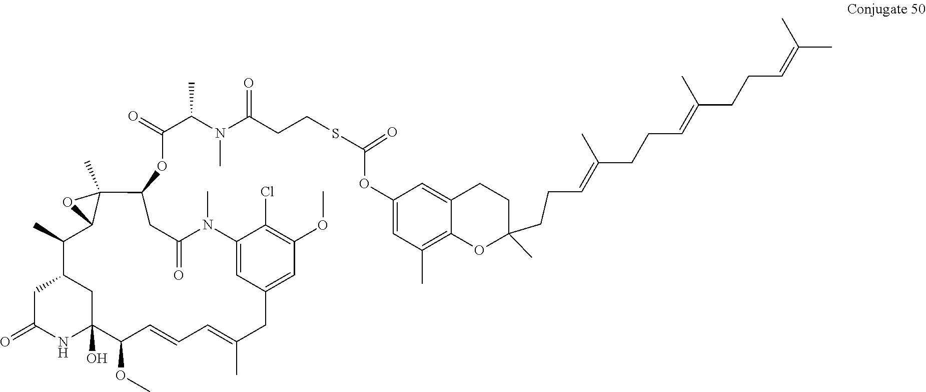 [SMILES: COc1cc2cc(c1Cl)N(C)C(=O)C[C@H](OC(=O)[C@H](C)N(C)C(=O)CCSC(=O)Oc1cc(C)c3c(c1)CCC(C)(CC/C=C(\C)CC/C=C(\C)CCC=C(C)C)O3)[C@]1(C)O[C@H]1[C@H](C)[C@H]1CC(=O)N[C@](O)(C1)[C@H](OC)/C=C/C=C(\C)C2]

### Example 3—Synthesis of delta-tocotrienol-gemcitibine conjugate with carbonate linker (Conjugate 60)

By following the procedure set forth above, the delta-tocotrienol-gemcitibine conjugate (60) could be made provided that the amino group is protected (e.g., Boc protected) before formation of the carbonate and then removed after formation of the carbonate to provide the structure set forth below:

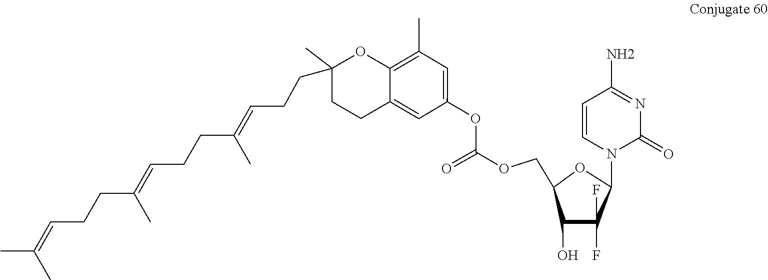

### Example 4—Synthesis of Delta-Tocotrienol-Gemcitibine Conjugate with Carbamate Linker (Conjugate 70)

This conjugate was prepared as per Example 19 of U.S. Pat. No. 10,532,105

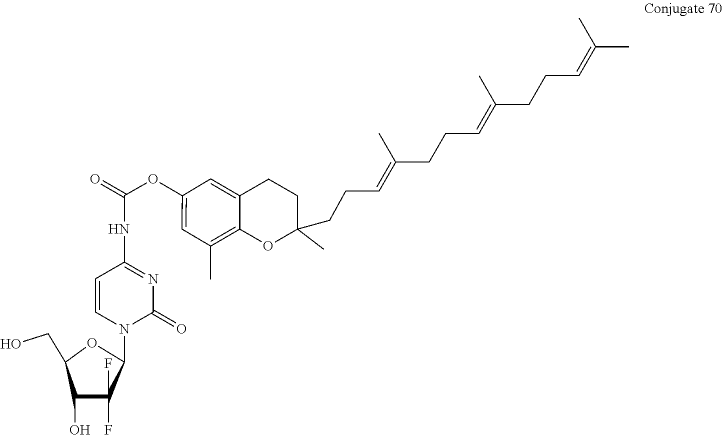

### Example 5—General Method for the Preparation of C-m-HDLs

A Flow Chart for the preparation of C-m-HDLs is provided in FIG. 15. The general method requires the preparation of Compositions A, B (aka Composition 1), C (aka Composition 2), the use of a 3-chamber reaction device shown in FIG. 4, dialysis (or tangential flow filtration (TFF) of the resulting suspension to remove ethanol and acid. In some embodiments, the resulting suspension is lyophilized.

Specifically, the preparation of Compositions A and B are found in Steps A and B; the preparation of Composition C is found in Step C; the mixing steps are in Steps D and E; dialysis (or TFF) is Step F; and the optional lyophilization procedure is found in Step G. For a particular non-limiting example, these steps are exemplified as follows:

A. Combine about 1.27 parts by weight of the peptide of SEQ ID NO. 35 into a solution comprising about 71.2 parts by weight ethanol, about 17.7 parts by weight sterile water, and about 5.8 parts by weight of glacial acetic acid and stir until the peptide is completely dissolved;

B. add about 2.35 parts by weight of POPC (commercially available from Lipoid, Newark, NJ) and about 0.95 parts by weight SM (commercially available from Lipoid, Newark, NJ), and about and the balance being Conjugate 20 of Example A (“Composition A”). Stir until clear. The combination of solutions from Step A and Step B is Composition A.

Next, filter the solution through a 0.4 μm (or in some cases a 0.2 μm) filter to remove insoluble particles and provide “Composition 1” or “B,” wherein the weight of all of the components added together, including the peptide, equals 100.

C. dissolve about 50 g of a sucrose into 1 liter of sterile water and stir until dissolved; filter the solution with a 0.2 μm filter (or in some cases a 0.4 μm filter) to remove insoluble particles. The filtered sucrose solution is defined as “Composition C” also referred to herein as Composition 2.

D. add Composition B to one holding chamber of a 3-chamber device comprising two holding chambers and one mixing or reaction chamber; and add Composition C into the second of two holding chambers;

E. with the reaction chamber held at 50° C., eject Compositions B and C from each holding chamber into the reaction chamber at a total flow rate (sum of B and C flow rates) of 1 ml/min to 10 ml/min, and wherein the ratio of the flow rate of Compositions B and C added to the reaction chamber ranges from i) about 1 part by volume of B to about 10 parts by volume of C to ii) about 1 part by volume of C to about 10 parts by volume of B. In some examples the ratio is from about 4:1 to about 10:1 of Composition C to Composition B. In further examples, the ratio is about 7:1 of Composition C to Composition B.

F. subject the solution comprising a clear suspension of nanoparticles to dialysis against about 2 to about 6 weight % and preferably about 3 to about 6 weight % sucrose to remove at least a portion of the ethanol and the acid, thereby providing for a composition as described herein. Alternatively, the resulting solution can be treated with tangential flow filtration against about 2 to about 6 weight % and preferably about 3 to about 6 weight % sucrose to reduce the amount of solvent followed by forming a concentrate using tangential flow filtration.

G. sterile filter the solution prepared in Step F through a filter having a 0.2 μm filter to provide for a sterile solution suitable for lyophilization which is assigned as Composition 20 (for conjugate 20); Composition 30 (for conjugate 30); Composition 40 (for conjugate 40); Composition 50 (for conjugate 50), Composition 60 (for conjugate 60); and Composition 70 (for conjugate 70).

As above, Composition 1 refers to the filtered solution formed by the addition of Composition B to Composition A followed by filtering after a clear solution is obtained. Composition 2 refers to the disaccharide solution.

As above, Composition A constitutes from about 94 to about 97 weight percent of Composition 1 whereas Composition B constitutes the remaining about 3 to about 6 weight percent of Composition 1.

As above, Composition A is stirred until clear. The addition of the acid is necessary to obtain a clear solution. When so prepared, Composition A will constitute about 94% to about 97% by weight of Composition 1 whereas Composition B will constitute about 3% to about 6% of Composition 1.

In some embodiments, the ranges of each component in Composition A (Composition 1) will vary depending on whether the lipid/conjugate used is at the minimum of 3 weight percent or at the maximum at about 6 weight percent. Table 9 provides the low range and high range components based on these extremes. Combinations within these extremes should fall within the weight percents shown.

TABLE 9
Weight Percent Range of each Component
of Composition A in Composition 1

| Compo- | | |
|---|---|---|
| nent | 94% Range | 97% Range/3% lipids |
| Ethanol | about 47.0% to about 84.6% | about 48.5% to about 87.3% |
| Water | about 5.0% to about 47.0% | about 7.3% to about 48.5%* |
| Acid | about 1.88% to about 09.4% | about 1.94% to about 9.70% |
| Peptide | about 0.94% to about 2.35% | about 0.97% to about 2.43% |
| POPC + | about 3.00% to about 4.2% | about 1.50% to about 2.10% |
| other | | |
| Lipids | | |
| SM | about 1.08% to about 1.80% | about 0.54% to about 0.90% |
| Conjugate | about 0.72% to about 2.10% | about 0.36% to about 1.05% |
| Benzyl | about 0% to about 7.5%** | about 0% to about 5%** |
| alcohol | | |

In the above tables, all percentages are weight percentages which are readily converted to parts by weight to 100 mg. The “*” refers to the fact that the total water present includes that contained with the 95% ethanol, any water associated with the acid used and the like. The “**” refers to the fact that benzyl alcohol may be added as needed to address remaining insolubility in Composition 1 depending on the character of the chemotherapeutic agent.

FIG. 4 illustrates a three-chamber mixing device suitable for use in this example. In some embodiments, the flow rates, and temperatures are computer controlled. The use of computers to control flow rates, temperatures, reaction times, etc. are well known in the art. In general, the mixing device includes a first chamber associated with a first pump, a second chamber associated with a second pump, and a third mixing chamber. A computer is configured to control the first and second pumps to provide for controlled fluid velocity from the first and second chambers into the mixing chamber at a controlled rate and ratio. One of the first and second pumps comprises the water solution comprising from about 2 to about 6 weight percent disaccharide such as sucrose as discussed herein.

Composition 20 comprises a suspension of C-m-HDLs in an aqueous formulation which particles have an average diameter of about 12 to about 13 nm.

By following these procedures, C-m-HDLs particles containing the Conjugates 20, 30, and 50 were individually prepared. C-m-HDL containing Conjugate 40 was made using a Composition A in which the liquid components consisted of 76 weight percent ethanol, 12 weight percent water, 6 weight percent acetic acid, and 6 weight percent benzyl alcohol. Each of the resulting C-m-HDLs were formed as a suspension in an aqueous formulation containing 3%-5% sucrose. The average diameter of C-m-HDLs was in the range of 12 to about 14 nm when measured by DLS.

An aqueous formulation of Composition 20 was assessed to determine whether it was suitable for prolonged storage at 4° C. However, after about 7 days, Composition 20 evidenced particle aggregation demonstrating the lack of long-term product stability of the solution when retained under refrigerated conditions.

### Example 6—Lyophilization-formation of Lyophilizate 20

In order to assess whether the C-m-HDLs compositions comprising a disaccharide were suitable for lyophilization, Composition 20 was subject to lyophilization. Specifically, after sterile filtration through a 0.2 μm filter, approximately 1 mL aliquots of a homogenous suspension of Composition 20 were lyophilized by placing the aliquots in suitable vials on the lyophilizer shelf. Lyophilization was initiated at a temperature of minus 38° C. maintained under a vacuum pressure of 50 mTorr for about 24 hours. Lyophilization was then continued for another 24 hours with the lyophilizer shelf temperature set to 20° C. Subsequently, the chamber was equilibrated to atmospheric conditions and the vials containing the lyophilized micellular nanoparticles were capped and designated as “Lyophilizate 20”.

### Example 7—Reconstitution of Lyophilizate 20

A sample vial of Lyophilizate 20 was reconstituted by addition of 1 ml of water such that the concentration of the micellular nanoparticles was the same as that found in the Composition 20 before lyophilization. An aliquot of reconstituted Lyophilizate 20 was diluted 10-fold (v/v) with standard phosphate buffered saline for analysis by dynamic light scattering. The resulting clear suspension of lyophilized micellular nanoparticles provided an average diameter of about 13 nm evidencing that lyophilization of the micellular nanoparticles had no material impact on the size of the diameter of these particles.

### Example 8—Alternative Methods to Determine the Diameter of the Micellular Nanoparticles

Gel permeation chromatography with a ThermoFisher SEC-1000 (Acclaim®) column was performed to compare the size of the micellular nanoparticles of Lyophilizate-20 against proteins of known Stokes diameters. The retention time for each of these particles relates to their average diameter and is used to confirm that the DLS values are verified. Gel permeation chromatography was conducted with a standard phosphate buffer saline mobile phase at 0.3 mL per minute under standard conditions. The results are provided in FIG. 3 which correlates retention time to the average size of the particles described therein and confirms an average diameter of 13 nm.

### Example 9—Assessing the Diameter of the Nanoparticles of the Reconstituted Composition

The diameter of the C-m-HDLs of Compound 20 was measured by size exclusion chromatography at the stages of a) pre-lyophilization, b) reconstitution in 0.89 weight % NaCl after lyophilization, and c) after storage of reconstituted sample at 4° C. for 5 days. The C-m-HDLs were prepared with SEQ ID NO:35 peptide using 2% sucrose and loaded with Conjugate 20. The relative size corresponding to the retention time of protein standards is indicated. As shown in FIG. 9, the C-m-HDLs so tested retained substantially the same average diameter. Note that the average diameter in this example is 13.9 nm using a 2% solution of sucrose which is about 0.8 nm larger than that obtained using 5% sucrose.

### Example 10—the Impact of Different Amounts of Disaccharide on the Average Diameter of the Nanoparticles

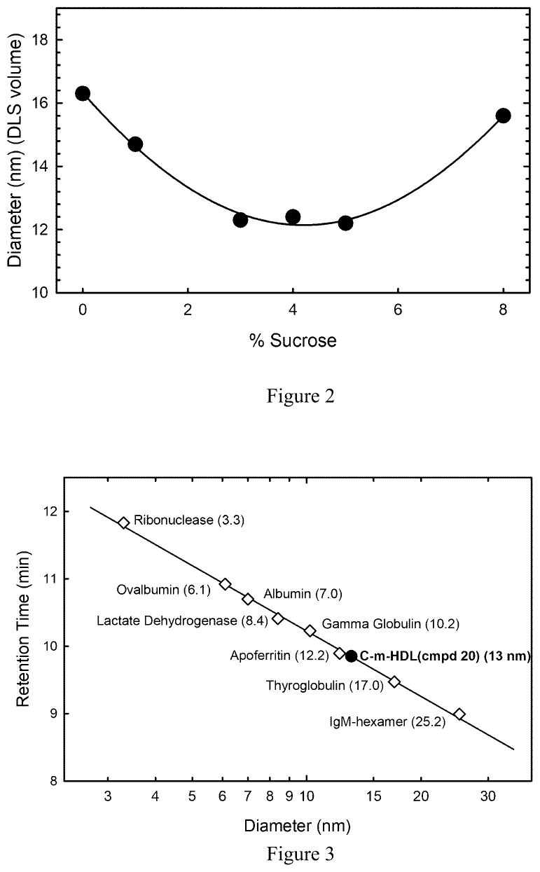

C-m-HDLs of Conjugate 20 were prepared as above under identical conditions but for the amount of sucrose employed. Specifically, five (5) different compositions of sucrose were prepared each having a different amount of sucrose in the aqueous solution used in the dual mixer. These variable amounts are as follows: 0 percent by weight, 1 percent by weight, 3 percent by weight, 5 percent by weight, and 8 weight percent. The results of that analysis are shown in FIG. 2.

As shown in FIG. 2, at 0% sucrose, the average diameter was approximately 17 nm. However, using about 3, 4 or 5 weight percent of sucrose in Composition C corresponded to an average diameter of about 12-13 nm. However, at 8 weight percent sucrose, the average diameter size increases to about 15 nm which may be attributed to increased solution viscosity.

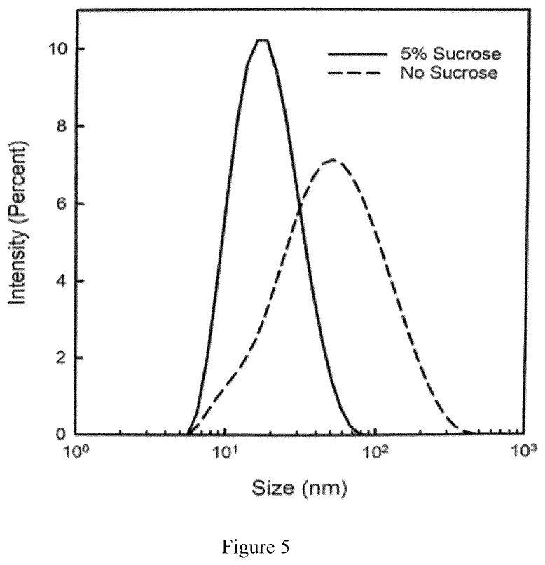

In addition, FIG. 5 shows the DLS results for the volume-based size distribution of C-m-HDL (cmpd 20) made without sucrose as compared to made with aqueous solution containing 5% sucrose. The results of this comparison evidence a much smaller average diameter for nanoparticles prepared with sucrose compared to those made with no sucrose. Likewise, the distribution width for the sucrose-made nanoparticles was narrower than that for those made without sucrose.

### Comparative Example A—Impact of nanoparticle size of efficacy with a solid mass tumor

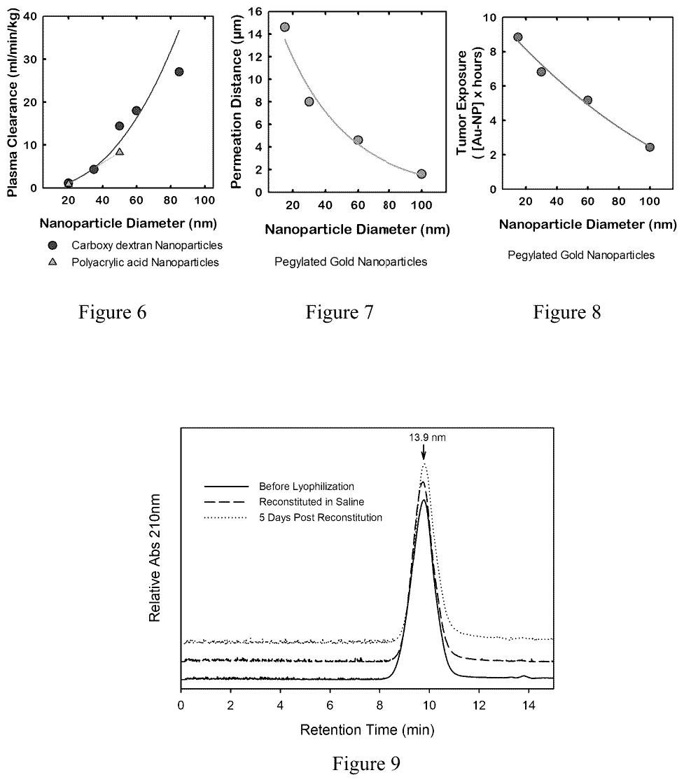

This example is taken from the literature which shows correlation as to the extent of penetration of nanoparticles into a solid mass tumor with the size of the nanoparticles. The purpose of this comparative example is to illustrate the improved extent of penetration of the nanoparticles described herein into the tumor and the corresponding impact on the overall efficacy of therapeutic nanoparticles. FIGS. 6, 7, and 8 address the findings from this endeavor. As to FIG. 6, it illustrates data showing the extent of plasma half-life of nanoparticles in general based on their size but not their contents; FIG. 7 illustrates data showing the extent of depth of tumor penetration of nanoparticles based on their size; and FIG. 8 illustrates data from a literature reference showing the extent of duration of tumor exposure of nanoparticles based on their size. See, e.g., Rooh, et al., Inter. J. Nanomedicine, 7:4447-4456 (2012); and Sykes, et al., ACSNano, 8 (6): 5696-5706 (2014) both of which are incorporated herein by reference in their entirety. Taken together, the data demonstrates that efficacy is improved by making nanoparticles as small as possible.

### Example 11—a Further Example for the Preparation of C-m-HDLs-20

This example is a further illustration for the preparation of C-m-HDLs using conjugate 20 in the nanoparticles. Specifically, 64 mg of POPC, 25.7 mg of SM, 34.6 mg of Seq ID NO. 35 peptide, and 13.4 mg of Conjugate 20 were dissolved in 3 mL of a solvent mixture, consisting of 75% ethanol, 20% water and 5% glacial acetic acid (vol %), to form an ethanolic solution for microfluidic mixing. The ethanolic solution was pumped from a syringe pump at 0.5 mL/min into the center mixer inlet of a Dolomite Microfluidics micromixer chip (PN-3200401) which is a three-chamber device where the syringe pump with the ethanolic solution is a first chamber. Simultaneously, a second syringe pump, the second chamber, delivered 4% (W/V) sucrose in water at 3.5 mL/min total flow rate to both outer mixer inlets where the mixer inlet is the third chamber. The chip temperature was maintained at 50° C. The water and solvent mixture emerging from the mixer chip was collected and dialyzed (10 kD MWCO, cellulose ester) against 10 mM Tris, 4% sucrose pH 7.4 to introduce Tris buffer and remove ethanol and acetic acid. The volume of the dialysis retentate was reduced 6- to 8-fold with a spin-filter (10 kD MWCO, PES) centrifuged at 2000× gravity. The retentate was distributed among glass vials, frozen, and lyophilized for 24 hr at a shelf temperature of −40° C., followed by 24 hr lyophilization at a shelf temperature of 25° C. and stored for future use.

### Example 12—Comparing the Growth Inhibition of SK-OV-3 Cells by C-m-HDLs Loaded with Conjugate 20 or Conjugate 30

In this Example, the mole portions of components in the C-m-HDLs prepared by solvent lyophilization were POPC: egg SM: peptide: conjugate 5.6:2.4:2:1. 20 mM stock solutions of POPC and of egg SM were prepared in tert-butyl alcohol: water (8:2 v/v). Peptide SEQ ID NO: 25 (acetate counter ion) was dissolved in tert-butyl alcohol/water/acetic acid (80:20:7 v/v) to obtain a 10 mM peptide solution. Conjugates 20 and 30 were separately dissolved in tert-butyl alcohol: water (95:5) to a final concentration of 10 mM.

For C-m-HDL preparation, 2.8 mL of 20 mM POPC, 1.2 mL of 20 mM egg SM, 2 mL of 10 mM peptide, 1 mL of 10 mM conjugate, and 0.3 mL acetic acid were combined in a 20 mL glass vial. The combined solution was frozen at ≥−70° C. for ≥1 hr. The frozen vial was placed in the chamber of a lyophilizer set at a shelf temperature of −20° C. A vacuum between 100 to 150 mTorr was applied for ≥20 hr. The shelf temperature was raised to −10° C. and the vacuum was continued for ≥15 hr. The shelf temperature was then raised to 20° C. and the vacuum continued for 24 hr. The dried cake was dissolved in PBS to obtain C-m-HDLs. The uniformity of the C-m-HDLs was evaluated by SEC (Acclaim™ SEC-1000, 300 mm×4.6 mm column, 0.3 mL/min PBS mobile phase, 210 nm and 280 nm UV detection) and by dynamic light scattering. The phospholipid content was established with a commercially available, enzyme-based, colorimetric assay kit. Peptide and conjugate content were established by HPLC.

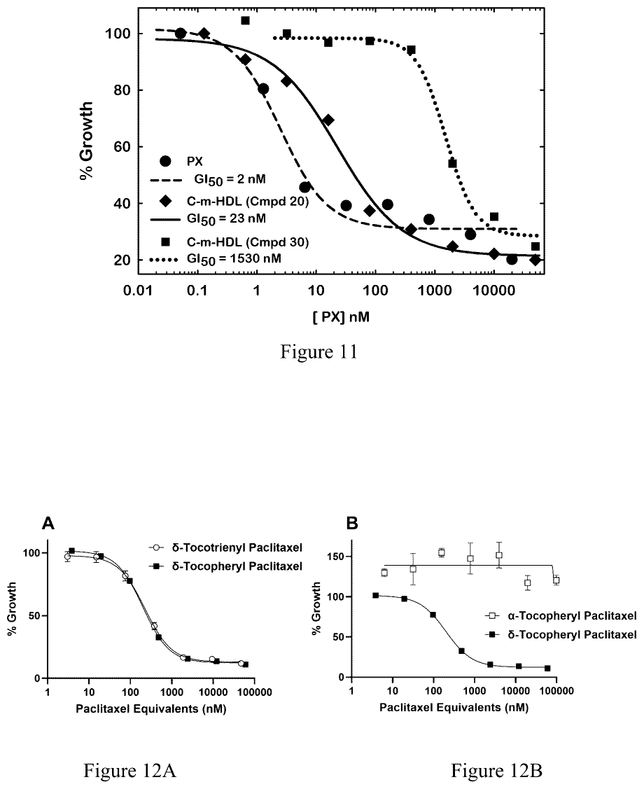

Two C-m-HDLs were prepared as above, containing either Conjugate 20 or Conjugate 30 were then tested for their growth inhibition activity against SK-OV-3 cells (ATCC). SK-OV-3 cells were grown in McCoy's-5A media supplemented with 10% fetal bovine serum and maintained in a 37° C. incubator under a 5% CO₂ atmosphere. To assess growth inhibition, the growth medium of cells grown in 96-well plates was supplemented with various concentrations of C-m-HDL (cmpd 20), C-m-HDL (compd 30) or paclitaxel from a DMSO stock solution. The 96-well plates were incubated for 72 hr. The relative amount of live cells in each of the wells after 72 hr was determined with the MTT assay. The concentrations of test articles causing 50% growth inhibition (GI₅₀) were obtained by performing a non-linear fit of the MTT results to a 4-parmeter logistic function. The results are plotted in FIG. 11. The results set forth in FIG. 11 evidence that both C-m-HDL (cmpd 20) and C-m-HDL (cmpd 30) effectively reduced tumor cell growth, but that growth inhibition was greater with C-m-HDL (cmpd 20).

### Example 13—Assessing Adrenal Gland Functionality in Rats after Dosing with the HDL Mimetic Chemotherapeutic Micellular Nanoparticles

A paclitaxel stock solution was prepared by dissolving paclitaxel in 1:1 (v/v) Cremophor EL/ethanol (95%) to a final paclitaxel concentration of 2.67 mg/mL (C-E paclitaxel solution). Next, a paclitaxel solution suitable for dosing was prepared by combining one volume of the C-E paclitaxel solution with 3 volumes of 0.9 weight % NaCl. This solution is designated as “Control”.

C-m-HDL (cmpd 20) was prepared as per Example 12.

The in vivo study was conducted in rats in accordance with the current guidelines for animal welfare (Guide for the Care and Use of Laboratory Animals, 8th Ed., 2011). The procedures used in the study were reviewed and approved by the Institutional Animal Care and Use Committee.

Eighteen (18) female Sprague-Dawley rats were randomly assigned to 3 groups of 6 animals each. All animals were lightly anesthetized using isoflurane and dosed intraperitoneally on Days 1, 4, and 7 with paclitaxel at 4 mg/kg in a dose volume of 1.5 mL/kg, or intravenously with C-m-HDL (cmpd 20) at 4 mg/kg or vehicle (saline) via the tail vein using a dose volume of 1.5 mL/kg. Group I received saline, Group 2 received paclitaxel, and Group 3 received C-m-HDL (cmpd 20). On Day 11, rats in each group were lightly anesthetized using isoflurane within their home cage to minimize potential stress responses. Blood (˜200 μL) was collected into K3EDTA tubes prior to ACTH challenge (baseline). Rats were then challenged with a single intraperitoneal injection of ACTH (ACTH 1-24, Sigma-Aldrich A0298-1922) in normal saline at 25 g/kg in a dose volume of 2 mL/kg. Blood samples (˜200 μL) were collected under isoflurane anesthesia into K3EDTA tubes at 1, 2, and 4 hours post ACTH challenge. Samples were rendered to plasma and stored at −80° C. pending corticosterone analysis. Plasma corticosterone content was determined with a commercially available ELISA kit.

The resulting data is provided in FIG. 10 which evidence that administration of C-m-HDL (cmpd 20) had no negative impact on adrenal functional as compared to control.

The data in the above example demonstrate that the adrenal glands are agnostic to paclitaxel as a cell toxin which accounts for the results achieved.

Likewise, there was no evidence of liver toxicity in male or female Spraque-Dawley rats dosed QW×4 with 40 mg/kg paclitaxel equivalents as found in C-m-HDL (cmpd 20) (Table 10). Lack of liver toxicity was based on finding no elevation in alanine transaminase and total bilirubin at the end of dosing, compared to control rats as per Table 10 below.

TABLE 10
Comparison of Abraxane ® and C-m-HDL(Cmpd
20) Toxicity in Sprague-Dawley Rats

| | | | Multiple Dose Tolerance | |
|---|---|---|---|---|
| | Single Dose Tolerance | | (4 weekly doses) | |
| Adverse | | C-m-HDL | | C-m-HDL |
| Observation | Abraxane ® | (Cmpd 20) | Abraxane ® | (Cmpd 20) |
| Lethality | At 50 mg/kg | None at | At 4 × 7 | None at |
| | | 64 mg/kg | mg/kg | 4 × 40 mg/kg |
| Body Weight | At 50 mg/kg | None at | At 4 × 7 | None at |
| Loss | | 64 mg/kg | mg/kg | 4 × 40 mg/kg |
| Anemia | At 50 mg/kg | None at | — | None at |
| | | 64 mg/kg | | 4 × 40 mg/kg |
| Liver | — | — | At 4 × 16 | None at |
| Enzymes | | | mg/kg | 4 × 40 mg/kg |

Reported in Food and Drug Administration NDA 21,660 Pharmacology/Toxicology Review and Evaluation

### Example 14—Inhibition of SK-OV-3 Xenograft Growth in Nude Mice by Conjugate 20 Delivered via C-m-HDLs

The C-m-HDL (cmpd 20) was composed of SEQ ID NO:35 peptide, POPC, SM, and Conjugate 20, in a mass ratio of 26:42:17:15 respectively which was prepared by dissolving the components in ethanol: water: acetic acid (75:20:5 vol ratio) to a final POPC content of 30 mM. The ethanolic solution was delivered at 0.5 mL/min to one input channel of a micro-mixing chip heated to 50° C., as described above. A solution of 1% trehalose in water was delivered at 3.5 mL/min to the second input channel. The effluent was transferred to dialysis tubing (10 kD MW cutoff) and dialyzed against 1% trehalose to remove the solvents. The dialysate was concentrated with 10 kD MW cutoff spin filters to 5 mg/mL paclitaxel equivalents and sterilized by passage through a 0.2 μm pore size PES filter.

For dosing, the C-m-HDL test solution were diluted with 0.87% NaCl to the concentration needed for dosing in a volume≤1% body weight. Paclitaxel dosing solution was prepared by combining four volumes of water with one volume of paclitaxel in Cremophor/ethanol (1:1 v/v) (Paclitaxel test composition). The vehicle control group received only 0.87% NaCl.

All animal procedures and maintenance were conducted in accordance with all the laws, regulations, and guidelines of the National Institutes of Health (NIH). The procedures used in the study were reviewed and approved by the Institutional Animal Care and Use Committee.

Female nude mice (HSD: NU-Foxnlnu) at 9-10 weeks of age were fed immunocompromised mouse diet and water ad libitum. Mice were housed in ventilated cages (≤ 5 mice/cage) with irradiated corncob bedding. All treatments, body weight determinations, and tumor measurements were carried out in a laminar flow bio-safety cabinet. The environment was controlled to a temperature range of 70°+2° F. and a humidity range of 30-70%. All mice were observed for clinical signs at least once daily.

The animals were randomly assigned to three cohorts-Vehicle control Group 1; Paclitaxel Test Composition Group II; and C-m-HDL-conjugate 20 Test Composition Group 3. Each animal in each cohort was then injected with SK-OV-3 cells which were obtained from the ATCC and expanded using McCoy's-5A media supplemented with 10% fetal bovine serum in a 5% CO₂ atmosphere at 37° C. The cells were collected and pooled for implantation as a suspension of 1×10^{7lls/mL in a solution of Matrigel/saline (}1:1) and kept on ice until use. Test mice (15 to 17 g) were implanted subcutaneously in the region of the right flank on Day 0 with 1×10⁶ cells/mouse (0.1 mL) using a small-gauge needle and syringe.

Treatments with test articles began on Day 5, when the mean estimated tumor mass for all groups in the experiment was 35 mg (range of group means, 34-37 mg). Mice were dosed according to individual body weight on the day of treatment (0.2 mL/20 g). Body weights and tumor measurements were recorded at least once weekly.

The results set forth in FIG. 14 evidence that the C-m-HDLs (cmpd 20)—at 30 mg/kg paclitaxel equivalents resulted in reducing tumor growth as compared to Vehicle and equivalent to paclitaxel at 30 mg/kg.

### Example 15—C-m-HDLs made with different Peptides

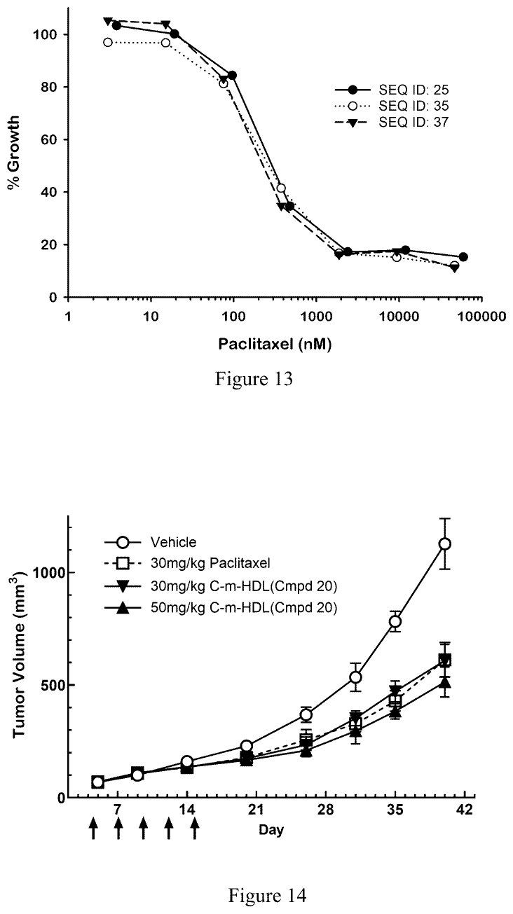

This example illustrates that C-m-HDLs using any one of SEQ ID NOs: 25, 35 or 37 provide binding to SR-BI and also provide for equivalent results when tested in vitro against ovarian adenocarcinoma SK-OV-3 cells as described above. The results of this example are provided in FIG. 13 wherein the curves for all three peptides used in otherwise the same C-m-HDLs were substantially the same.

### Example 16—Examples of Conjugates of paclitaxel and diverse anchor molecules

This example illustrates combinations of different lipids with paclitaxel can be used herein to generate a library of diverse conjugates as follows:

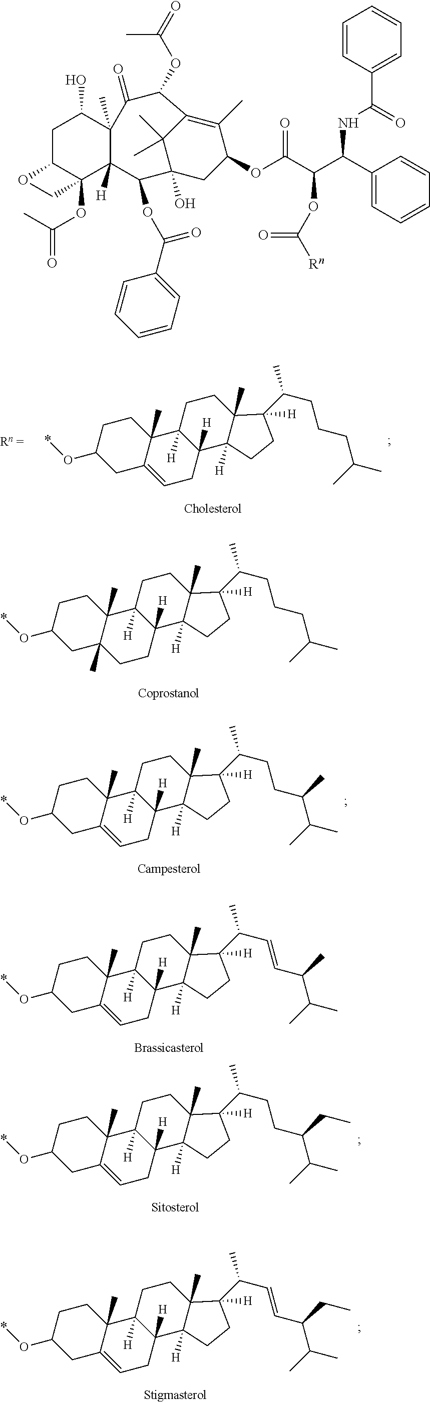

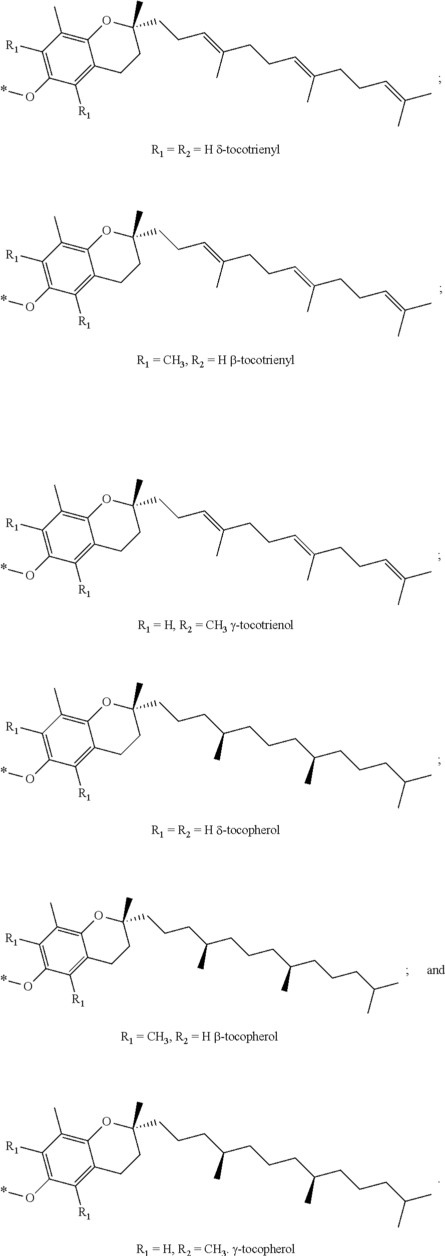

The following chemotherapeutic agents can be used in addition to paclitaxel merely by substitution. These include by way of example only, mertansine, exatecan, gemcitabine, eribulin, bendamustine, chlorozotocin, capecitabine, melphanine, streptzotocin, mitoxantrone, hydroxy camptothecin, troxacitabine, vincristine, sirolimus, tubulysin A, docetaxel, and cytarabine.

### Example 17—alpha Tocotrienol

This example illustrates compares the activity of C-m-HDLs made using a conjugate of paclitaxel-delta-tocotrienol versus a conjugate of paclitaxel-delta-tocopherol and both tested against alpha tocopherol. The tests were conducted as above, and the results of this comparison are found in FIGS. 12A and 12B.

In FIG. 12A, the activity of paclitaxel-delta-tocotrienol and paclitaxel-delta-tocopherol were essentially equivalent. However, in FIG. 12-B, the growth inhibition of SK-OV-3 cells by C-m-HDLs prepared by solvent lyophilization with SEQ ID NO:25 peptide and loaded alpha-tocopherol showed no activity. It is contemplated that perhaps the intracellular enzymatic cleavage of the carbonate bond between the alpha-tocopherol and paclitaxel is blocked by steric hinderance due to the trimethyl substitution pattern on the phenyl ring. It is contemplated that replacing the oxygen of tocopherol in the carbonate bond with the sulfur atom of mertansine will relieve the steric hinderance and permit intracellular enzymatic degradation.

### Example 18—Inhibition of SK-OV-3 cell growth C-m-HDLs of Example 1

A stock solution of 500 μM exatecan mesylate solution was prepared in 100% DMSO and then diluted 1:1250 with complete growth medium to obtain 0.4 μM exatecan. The solution was sterile filtered through a nylon membrane (0.2 μm). A similar process was used for preparation of C-m-HDLs of Example 18 test solutions. A stock of C-m-HDLs of conjugate 40 of Example 1 containing 0.8 mM C-m-HDLs of conjugate 40 in 3% sucrose, 10 mM TRIS, pH 7.4 in 0.9% saline was diluted to 100 μM in complete growth medium followed by filter sterilization through a PES filter (0.2 μm). This yielded the two-fold concentration of the highest final concentration tested, 50 μM. Further dilutions of these 2×test solutions were made in dilution increments of 5× in growth medium.

SK-OV-3 ovarian adenocarcinoma cells (American Type Culture Collection, HTB-77) were seeded in 96-well plates at a density of 10,000 cells per well (100 μL) and the seeded plates incubated overnight in a humidified tissue culture incubator (24 hour) in complete growth medium composed of McCoy's 5a medium supplemented with 10% fetal bovine serum.

The following day an additional 100 μL of complete growth medium were added to the blank and no-treatment cell containing wells. A 100 μL volume of the test media containing 2× exatecan or C-m-HDLs of were added to treatment wells, using 4 wells per concentration tested. Cells were incubated with the test solutions for 72 hours. At the end of this period cell viability was determined by MTT assay.

### Example 20—Inhibition of SK-OV-3 cell growth by C-m-HDLs containing Conjugate 50

A test solution of 200 nM mertansine in complete growth medium was prepared in two steps. A 10 mM stock solution of mertansine in 100% DMSO was first diluted 1:20 with 100% DMSO and then 1:2500 with complete growth medium yielding the twofold top concentration of 200 nM. This solution was filter sterilized through a 0.2 μM PES filter.

A 200 nM solution of C-m-HDLs containing conjugate 50 test solution in complete growth medium was prepared by diluting a 520 μM solution of C-m-HDLs containing conjugate 50 in 3% sucrose, 10 mM TRIS, pH7.4 in 0.9% saline to 200 nM concentration in complete medium. This was filter sterilized through a 0.2 μM polyethylsulfone filter. Further dilutions of these test solutions were made in dilution increments of 5× in growth medium.

SK-OV-3 ovarian adenocarcinoma cells (American Type Culture Collection, HTB-77) were seeded in 96-well plates at a density of 10,000 cells per well (100 μL) and the seeded plates incubated overnight in a humidified tissue culture incubator (24 hour) in complete growth medium composed of McCoy's 5a medium supplemented with 10% fetal bovine serum.

The following day an additional 100 μL of complete growth medium were added to the blank wells and wells containing no-treatment cells. A 100 μL volume of the various test media containing mertansine or C-m-HDLs containing Conjugate 50 (referred to as “cmpd 50” in FIG. 17) were added to treatment wells, using 4 wells per concentration tested. Cells were incubated with the test solutions for 72 hours. At the end of this period cell viability was determined by MTT assay.

### Example 19—Conjugates for Use Herein

A set of conjugates for use herein are provided in FIGS. 18A through 18N. Each of these conjugates illustrate δ-tocotrienol, δ-tocopherol, or cholesterol as the anchor moiety. It is understood that such is for illustrative purposes only and that other anchor moieties can be used in place thereof including but not limited to β-tocotrienol, γ-tocotrienol, β-tocopherol, γ-tocopherol, coprostanol, β-sitosterol, sitostanol, stigmasterol, stigmastanol, campesterol, brassicasterol, ergosterol, retinol, cholecalciferol, and ergocalciferol.

The above examples are illustrative in nature and, as such, are non-limiting. Because the conjugates of Examples A and B are disclosed in the art, these conjugates are not listed numerically.

### ILLUSTRATIVE EMBODIMENTS

Embodiment 1: A population of micellar nanoparticles comprising: (a) a peptide or protein that is mimics the SR-BI binding site of high density lipoprotein (HDL) on tumor cells; (b) a lipid; and (c) a conjugate comprising a chemotherapeutic agent and an anchor moiety which are attached to each other by a cleavable bond, wherein the C-m-HDLs comprises: a hydrophilic exterior surface; and a hydrophobic core comprising the conjugate; wherein the population of said micellular nanoparticles has an average particle diameter of about 11.5 to about 14 nanometers.

Embodiment 2: The population of Embodiment 1, wherein the cleavable attachment of the chemotherapeutic agent and the anchor moiety is via an ester bond, a carbonate bond, a thiocarbonate bond, a carbamate bond and a thiocarbamate bond.

Embodiment 3: The population of Embodiment 1 or 2 wherein said anchor moiety attached to said chemotherapeutic agent by a cleavable bond is absorbable into a tumor cell by SR-BI expressed by said cell.

Embodiment 4: The population of Embodiment 1, 2 or 3, wherein the peptide is an amphiphilic, alpha-helical peptide.

Embodiment 5: The population of Embodiment 1, 2 or 3 wherein the peptide is any one of SEQ ID NOs: 25, 28, 34, 35 or 36 or combinations thereof.

Embodiment 6: The population of Embodiment 5 wherein the peptide comprises an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence set forth in any one of SEQ ID NOs: 25, 28, 34, 35 or 36.

Embodiment 7: An aqueous composition comprising water, a mono-, di- or trisaccharide, and a population of micellular nanoparticles (C-m-HDLs) which comprises: (a) a peptide; (b) a lipid; (c) a conjugate comprising a chemotherapeutic agent and an anchor moiety, wherein the anchor moiety is covalently attached to said chemotherapeutic agent by a cleavable bond selected from an ester bond, a carbonate bond, a thiocarbonate bond, a carbamate bond and a thiocarbamate bond; wherein the population of C-m-HDLs comprises: a hydrophilic exterior surface; and a hydrophobic core comprising the conjugate; wherein the C-m-HDLs have an average particle diameter of about 12.0 to about 13.5 nanometers; wherein the peptide is selected from an amino acid sequence as set forth in SEQ ID NOs: 25, 28, 34, 35 or 36.

Embodiment 8: The population of Embodiment 7, wherein said composition further comprises a disaccharide selected from sucrose, lactose, maltose, trehalose, cellobiose or lactulose.

Embodiment 9: The population of Embodiment 8, wherein the disaccharide is sucrose.

Embodiment 10: A population of C-m-HDLs which comprises: (a) an amphiphilic, alpha-helical peptide selected from a peptide having an amino acid sequence of SEQ ID NO:25, SEQ ID NO:28, SEQ ID NO:34, SEQ ID NO:35 or SEQ ID NO:36; (b) one or both of sphingomyelin and phosphatidyl choline, and optionally additional lipid(s) including phospholipid(s); and (c) a conjugate comprising a chemotherapeutic agent, and an anchor moiety which are covalently linked by a cleavable bond selected from an ester bond, a carbonate bond, a thiocarbonate bond, a carbamate bond and a thiocarbamate bond, wherein said C-m-HDLs in said population comprise a hydrophilic exterior surface; a hydrophobic interior surface defining hydrophobic core comprising the conjugate, one or both of sphingomyelin and phosphatidyl choline, and further wherein said C-m-HDLs in said population have an average particle diameter of from about 12 to about 13.5 nanometers, as measured by dynamic light scattering.

Embodiment 11: The population of C-m-HDLs of Embodiment 10, wherein at least about 65% of the m-HDLs-paclitaxel in said population are within plus/minus about 2 nanometers of the average diameter.

Embodiment 12: The population of C-m-HDL-s of Embodiment 10, wherein at least about 70% of the C-m-HDLs in said population are within plus/minus about 2 nanometers of the average diameter.

Embodiment 13: The population of C-m-HDLs of Embodiment 10, wherein at least about 75% of the C-m-HDLs-paclitaxel in said population are within plus/minus about 2 nanometers of the average diameter.

Embodiment 14: The population of C-m-HDLs-paclitaxel of Embodiment 10, wherein at least about 80% of the C-m-HDLs-paclitaxel in said population are within plus/minus about 2 nanometers of the average diameter.

Embodiment 15: A composition comprising a population of C-m-HDLs-paclitaxel of any one of Embodiments 1 through 14, wherein said composition is in an aqueous environment and said chemotherapeutic agent in said C-m-HDLs is selected from those set forth in Example 16.

Embodiment 16: A composition comprising a population of C-m-HDLs of any one of Embodiments 1 through 14, wherein said composition is a lyophilized.

Embodiment 17: A composition comprising a population of lyophilized C-m-HDLs according to Embodiment 16 wherein said disaccharide is sucrose.

Embodiment 18: The composition of Embodiment 17, wherein the concentration of sucrose in said C-m-HDLs-paclitaxel ranges from about 20 to about 300 weight percent based on the weight of the lyophilized-C-m-HDLS.

Embodiment 19: The composition of Embodiment 17, wherein the concentration of sucrose in said C-m-HDLs ranges from about 50 to about 150 weight percent based on the weight of the C-m-HDLs.

Embodiment 20: The composition of Embodiment 17, wherein the concentration of sucrose in said C-m-HDLs ranges from about 75 to about 125 weight percent based on the weight of the C-m-HDLs.

Embodiment 21: The population of C-m-HDLs according to any one of Embodiments 1-20, wherein the chemotherapeutic agent is attached to an anchor moiety via a carbonate group (as exemplified for paclitaxel which is used for illustrative purposes only):

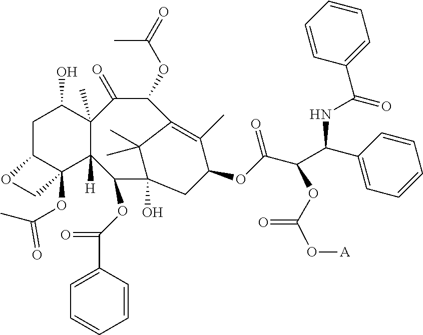

where A is the anchor moiety as per Embodiment 15.

Embodiment 22: The population of C-m-HDLs according to Embodiment 21, wherein the anchor moiety is cholesterol, cholecalciferol, β-, γ-, and δ-tocotrienol isoforms, or the β-, γ-, δ-tocopherol isoforms or combinations thereof.

Embodiment 23: The population of C-m-HDLs according to Embodiment 22, wherein the anchor moiety is cholesterol or δ-tocotrienol.

Embodiment 24: The population of C-m-HDLs according to Embodiments 1-14 and 16-20, wherein the conjugate is selected from anyone set forth in Tables 1 to 5 supra.

Embodiment 25: A conjugate for use in preparing the C-m-HDLs-cholesterol as described herein wherein said conjugate has the formula:

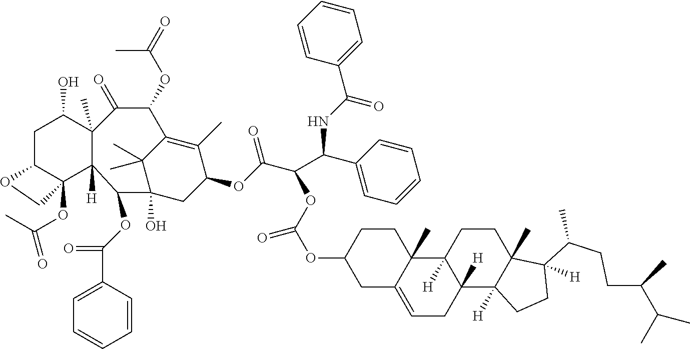

Embodiment 26: A conjugate for use in preparing the C-m-HDLs-paclitaxel as described herein wherein said conjugate has the formula:

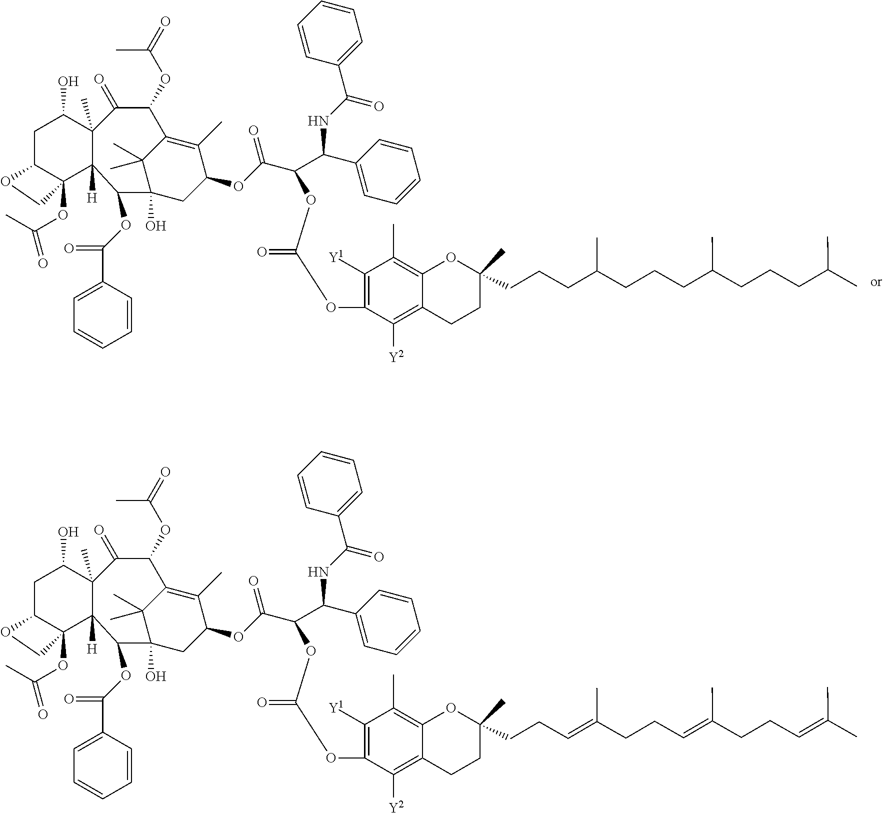

Y¹ and Y² are independently hydrogen or methyl provided that both can't be methyl.

Embodiment 27: A conjugate for use in preparing the C-m-HDLs-paclitaxel described herein wherein said conjugate has the formula:

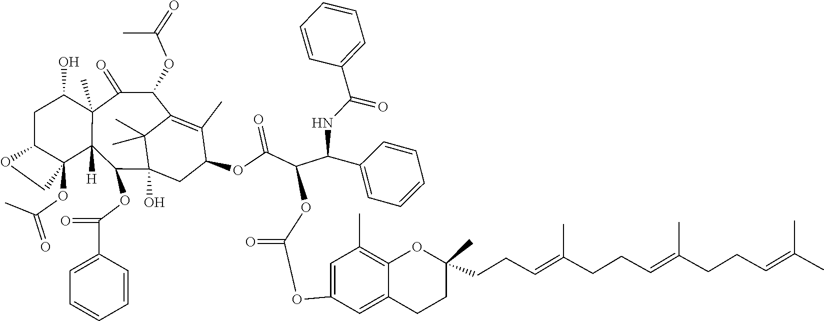 [SMILES: CC(=O)O[C@H]1C(=O)[C@@]2(C)[C@H]([C@H](OC(=O)c3ccccc3)[C@]3(O)C[C@H](OC(=O)[C@H](OC(=O)Oc4cc(C)c5c(c4)CC[C@@](C)(CC/C=C(\C)CC/C=C(\C)CCC=C(C)C)O5)[C@@H](NC(=O)c4ccccc4)c4ccccc4)C(C)=C1C3(C)C)[C@]1(OC(C)=O)CO[C@@H]1C[C@@H]2O]

or the formula:

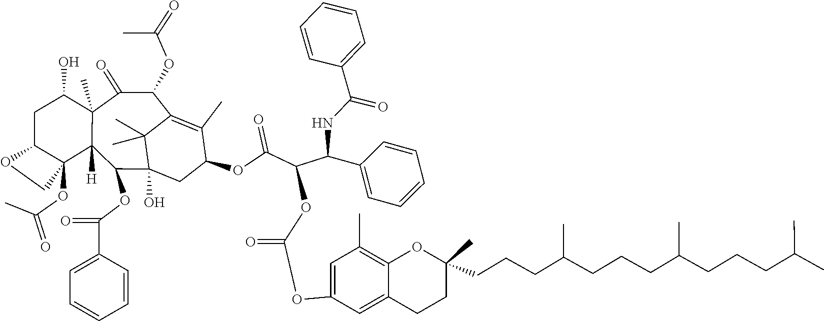 [SMILES: CC(=O)O[C@H]1C(=O)[C@@]2(C)[C@H]([C@H](OC(=O)c3ccccc3)[C@]3(O)C[C@H](OC(=O)[C@H](OC(=O)Oc4cc(C)c5c(c4)CC[C@@](C)(CCCC(C)CCCC(C)CCCC(C)C)O5)[C@@H](NC(=O)c4ccccc4)c4ccccc4)C(C)=C1C3(C)C)[C@]1(OC(C)=O)CO[C@@H]1C[C@@H]2O]

Embodiment 28: A method for preparing a solution of a fully dissolved amphiphilic, alpha-helical peptide selected from an amino acid sequence as provided by SEQ ID NO: 25, SEQ ID NO: 28, SEQ ID NO: 34, SEQ ID NO: 35, SEQ ID NO:36, and combinations thereof in an aqueous-ethanol co-solvent, which method comprises combining in any order:
a) from about 50 to about 90 weight percent ethanol and from about 7.5 (or about 10) weight percent to about 50 weight percent water;
b) adding from about 2 to about 10 weight percent of an acid based on the weight of a) above;
c) adding from about 1 to about 2.5 weight percent of an amphiphilic, alpha-helical peptide selected from an amino acid sequence as provided by SEQ ID NO: 25, SEQ ID NO: 28, SEQ ID NO: 34, SEQ ID NO: 35, and SEQ ID NO:36 based on the total weight of the solvent solution of b) above; and stirring until a clear solution is obtained.

Embodiment 29: A method for preparing a composition comprising C-m-HDLs-paclitaxel which method comprises: preparing a composition designated as “Composition 1”:
a) by combining in any order:
   i) ethanol and water to form a cosolvent;
   ii) acid;
   iii) a peptide of SEQ ID NO. 25, SEQ ID NO: 28, SEQ ID NO: 34, SEQ ID NO: 35, or SEQ ID NO: 36;
and stirring the composition until the solution is clear evidencing that the peptide is completely dissolved wherein the components described in a) comprise from 94 to 97 weight percent of Composition 1;
b) combine the solution generated in of a) with a lipid wherein said lipid composition comprises:
   iv) sphingomyelin;
   v) a conjugate as described herein; and
   vi) an additional lipid component (e.g., 1-palmitoyl-2-oleoyl-phosphatidylcholine);
   and stirring the resulting solution until it is clear and then filtering the composition; wherein the final composition comprises the following percentages of components:

| Compo- | | |
|---|---|---|
| nent | 94% Range | 97% Range/3% lipids |
| Ethanol | about 47.0% to about 84.6% | about 48.5% to about 87.3% |
| Water | about 7.05% to about 47.0% | about 7.3% to about 48.5%* |
| Acid | about 1.88% to about 09.4% | about 1.94% to about 9.70% |
| Peptide | about 0.94% to about 2.35% | about 0.97% to about 2.43% |
| POPC + | about 3.00% to about 4.2% | about 1.50% to about 2.10% |
| other | | |
| Lipids | | |
| SM | about 1.08% to about 1.80% | about 0.54% to about 0.90% |
| Conjugate | about 0.72% to about 2.10% | about 0.36% to about 1.05% |
| Benzyl | about 0% to about 7.5%** | about 0% to about 5%** |
| alcohol | | |

Embodiment 30: The composition of Embodiment 29 where the peptide aggregates to form an HDL mimetic structure that is recognized by SR-BI.

Embodiment 31-45: In these embodiments, representative amounts of each component in Composition 1 is provided in parts by weight of components based on a 100 g sample. These embodiments are representative of possible combinations and are as shown in Table 11:

TABLE 11

| | Peptide | | | | | Glacial | | |
|---|---|---|---|---|---|---|---|---|
| Embod. | SEQ. | | Compound 20 | | Sterile | Acetic | | |
| No. | ID | Peptide | (Conjugate) | Ethanol | Water | Acid | POPC | SM |
| 31 | 35 | 0.99 | 1.11 | 75.15 | balance | 5.66 | 2.17 | 1.02 |
| 32 | 35 | 1.23 | 2.00 | 65.70 | balance | 5.75 | 1.83 | 1.40 |
| 33 | 35 | 2.00 | 1.71 | 56.12 | balance | 8.12 | 3.60 | 1.80 |
| 34 | 35 | 1.93 | 0.92 | 77.72 | balance | 4.22 | 4.04 | 0.66 |
| 35 | 35 | 1.69 | 1.35 | 83.00 | balance | 2.11 | 1.90 | 1.74 |
| 36 | 28 | 1.11 | 0.50 | 53.30 | balance | 6.92 | 3.14 | 1.13 |
| 37 | 28 | 1.67 | 0.76 | 61.47 | balance | 9.14 | 2.97 | 1.67 |
| 38 | 28 | 1.81 | 1.97 | 72.33 | balance | 5.12 | 4.11 | 0.81 |
| 39 | 28 | 1.44 | 0.44 | 79.01 | balance | 4.98 | 2.97 | 1.21 |
| 40 | 28 | 1.29 | 1.66 | 83.33 | balance | 6.31 | 1.66 | 1.69 |
| 41 | 25 | 2.35 | 1.74 | 49.09 | balance | 6.78 | 3.88 | 0.88 |
| 42 | 25 | 1.13 | 1.47 | 58.57 | balance | 7.88 | 3.34 | 1.31 |
| 43 | 25 | 2.22 | 0.86 | 67.91 | balance | 3.79 | 2.75 | 1.53 |
| 44 | 25 | 2.11 | 0.71 | 74.47 | balance | 4.65 | 1.99 | 1.66 |
| 45 | 25 | 1.97 | 1.00 | 71.77 | balance | 5.11 | 2.35 | 1.47 |

Embodiment 46: A method for reducing the average diameter (size) of a population of C-m-HDLs which method comprises:
combining water with from about 2 to 6 weight percent of a disaccharide and mix until homogeneous and designated that solution as “Composition 2”;
adding Composition 2 to a first loading chamber of a three-chamber mixing device which comprises two separate loading chambers and a single reaction chamber;
adding Composition 1 of Embodiment 29 prepared as above into the other loading chamber of said two loading chambers in the device;
chaotically mixing the two compositions into the reaction chamber under a controlled flow rate ratio of from about 4:1 to about 10:1 of Composition 2 to Composition 1 at a temperature of from about 30° to about 70° C. while maintaining the flow from each chamber until mixing is complete while maintaining a temperature of from about 30° to about 70° C.;
thereby providing for a population of C-m-HDLs as a suspension in an aqueous solution where said population has an average diameter for the nanoparticles of about 11.5 to about 14 nanometers, and preferably about 12 nm to about 13.5 nanometers.

Embodiment 47: The method of Embodiment 46, wherein the C-m-HDLs have a distribution of particle sizes wherein at least about 65% of said particles are within +/−3 nm.

Embodiment 48: The method of Embodiment 46, wherein the C-m-HDLs have a distribution of particle sizes wherein at least about 70% of said particles are within +/−3 nm.

Embodiment 49: The method of Embodiment 46, wherein the C-m-HDLs have a distribution of particle sizes wherein at least about 75% of said particles are within +/−3 nm.

Embodiment 50: The method of Embodiment 46 wherein the C-m-HDLs have a distribution of particle sizes wherein at least about 80% of said particles are within +/−3 nm.

Embodiment 51: The method of Embodiment 46 wherein the acid employed is glacial acetic acid.

Embodiment 52: The method of Embodiment 51 which further comprises removing at least a portion of the ethanol and acetic acid by either dialysis or tangential flow filtration (TFF).

Embodiment 53: The method of Embodiment 52 wherein the resulting suspension is sterile filtered preferably using a 0.2 micron filter.

Embodiment 54: The method of Embodiment 53 which further comprises

lyophilizing the composition.

Embodiment 55: The method of Embodiment 46 wherein the disaccharide is selected from sucrose, lactose, maltose, trehalose, cellobiose and lactulose.

Embodiment 56: The method of Embodiment 55, wherein the disaccharide is sucrose.

Embodiment 57: The method of Embodiment 56, wherein the sucrose is used in an aqueous solution during manufacture of the micellular nanoparticles in an amount of about 2, or about 3, or about 4, or about 5, or about 6 weight percent sucrose based on the weight of the aqueous solution wherein said amount of sucrose includes all values between 2 and 6 weight percent based on 0.01 increments.

Embodiment 58: The method of Embodiments 55, 56 or 57, wherein the sucrose is used in an aqueous solution during manufacture of the micellular nanoparticles in an amount of about 3 or about 5 weight percent sucrose based on the weight of the aqueous solution.

Embodiment 59: The method of Embodiment 46, wherein the average diameter size of the C-m-HDLs after synthesis is reduced by about 10% as compared to the average diameter of C-m-HDLs made in the same manner but without sucrose.

Embodiment 60: The method of Embodiment 30, wherein the average diameter size of the C-m-HDLs after synthesis is reduced by about 15% as compared to the diameter of the C-m-HDLs made in the same manner but without sucrose.

Embodiment 61: The method of Embodiment 46, wherein the average diameter size of the C-m-HDLs after synthesis is reduced by about 20% as compared to the diameter of the C-m-HDLs made in the same manner but without sucrose.

Embodiment 62: The method of Embodiment 61, wherein average diameter size of the C-m-HDLs after synthesis is reduced by about 22% as compared to the diameter of the C-m-HDLs made in the same manner but without sucrose.

Embodiment 63: A method for treating a patient suffering from a solid mass tumor that overexpresses SR-BI which method comprises administering to said patient an effective amount of a population of C-m-HDLs of Embodiments 1 through 27.

Embodiment 64: The method of Embodiment 63 wherein the anchor moiety is transported from the C-m-HDLs into a tumor cell.

Embodiment 65: The method of Embodiment 64 wherein the cells are solid mass tumor cells.

Embodiment 66: The method of Embodiment 64, wherein the conjugate used in preparing the C-m-HDLs described herein comprises an anchor moiety and paclitaxel.

Embodiment 67: The method of Embodiments 63, 64 or 65, wherein the amount of disaccharide in the C-m-HDLs ranges from about 20 weight percent to about 300 weight percent disaccharide based on the weight of the C-m-HDLs.

Embodiment 68: The method of Embodiment 63, wherein the amount of disaccharide in the C-m-HDLs ranges from about 50 weight percent to about 150 weight percent disaccharide based on the weight of the C-m-HDLs.

Embodiment 69: The method of Embodiment 63, wherein the amount of disaccharide in the C-m-HDLs ranges from about 75 weight percent to about 125 weight percent disaccharide based on the weight of the nanoparticles.

Embodiment 70: The population or composition of any of Embodiments 1-27, wherein said population or said composition further comprises a pharmaceutically acceptable excipient.

Embodiment 71: The population or composition of any of Embodiments 1-27, wherein said population or said composition is an aqueous composition suitable for intravenous injection.

Embodiment 72: The aqueous composition of Embodiment 71 wherein said aqueous composition is reconstituted form a lyophilized population or composition of paclitaxel based micellular nanoparticles.

Embodiment 73: A pharmaceutical composition comprising a reconstituted aqueous composition suitable for intravenous injection which composition comprise water, sucrose and an effective amount of a population of C-m-HDLs which itself comprises: (a) an amphiphilic, alpha-helical peptide that comprises an amino acid sequence of SEQ ID NO:25, SEQ ID NO:28, SEQ ID NO:34, SEQ ID NO:35, or SEQ ID NO:36, (b) one or both of sphingomyelin and phosphatidyl choline, and optionally additional lipid(s) including phospholipid(s); and (c) a conjugate comprising chemotherapeutic agent and a lipid, wherein the lipid is releasably attached to the chemotherapeutic agent through an ester, thioester, carbonate, thiocarbonate, carbamate, or a thiocarbamate bond; wherein said C-m-HDLs comprise: a hydrophilic exterior surface; a hydrophobic core comprising the conjugate; wherein each of the micellar nanoparticles in the population of micellar nanoparticles has an average particle diameter of about 12-13.5 nanometers, as measured by dynamic light scattering.

Embodiment 74: The pharmaceutical composition as provided in Embodiment 73, wherein at least about 65% of the C-m-HDLs are within plus/minus about 3 nanometers of the average diameter.

Embodiment 75: The pharmaceutical composition as provided in Embodiment 73, wherein at least about 70% of the C-m-HDLs are within plus/minus about 3 nanometers of the average diameter.

Embodiment 76: The pharmaceutical composition as provided in Embodiment 73, wherein at least about C-m-HDLs of the nanoparticles are within plus/minus about 3 nanometers of the average diameter.

Embodiment 77: The pharmaceutical composition as provided in Embodiment 73, wherein at least about 80% of the C-m-HDLs are within plus/minus about 3 nanometers of the average diameter.

Embodiment 79: A kit of parts comprising a first vial comprising a unit dose of a lyophilized composition of Embodiment 16, a second vial comprising a unit amount of a sterile aqueous composition, and optionally a needle syringe.

Embodiment 80: The kit of parts of Embodiment 79, wherein said kit further comprises a product insert with instruction for use.

Embodiment 81: A method for treating a patient with a disorder mediated at least in part by the overexpression of SR-BI which method comprises administering to said patient an effective amount of a composition comprising C-m-HDLs of claim 1.

Embodiment 82: The method of Embodiment 81, wherein said disorder is a solid mass tumor that overexpresses SR-BI.

Embodiment 83: The method of Embodiment 82, wherein said solid mass tumor is selected from breast cancer (including triple negative breast cancer), bladder cancer, gastrointestinal cancers, head and neck cancers, neuroblastoma, non-small-cell lung cancer, ovarian cancer, pancreatic cancer, prostate cancer, stomach cancer, kidney cancer, and cervical cancer.

Embodiment 84: The method of Embodiment 83, wherein said solid mass tumor is breast cancer, non-small cell lung cancer, prostate cancer, ovarian cancer or cervical cancer.

Embodiment 85: The method of Embodiments 83 and 84, wherein said composition is administered to said patient as an intravenous solution.

Embodiment 86: The method of any of Embodiment 85, wherein said intravenous composition is an aqueous formulation reconstituted from a lysophilizate.

Embodiment 87: The method of Embodiments 85 and 86, wherein said composition is a pharmaceutical composition comprising a pharmaceutically acceptable excipient and about 5 mg/mL of paclitaxel equivalents in about a 4% or 5% by weight sucrose solution.

Embodiment 88: The method of Embodiment 81, wherein said conjugate employed in said composition is selected from:

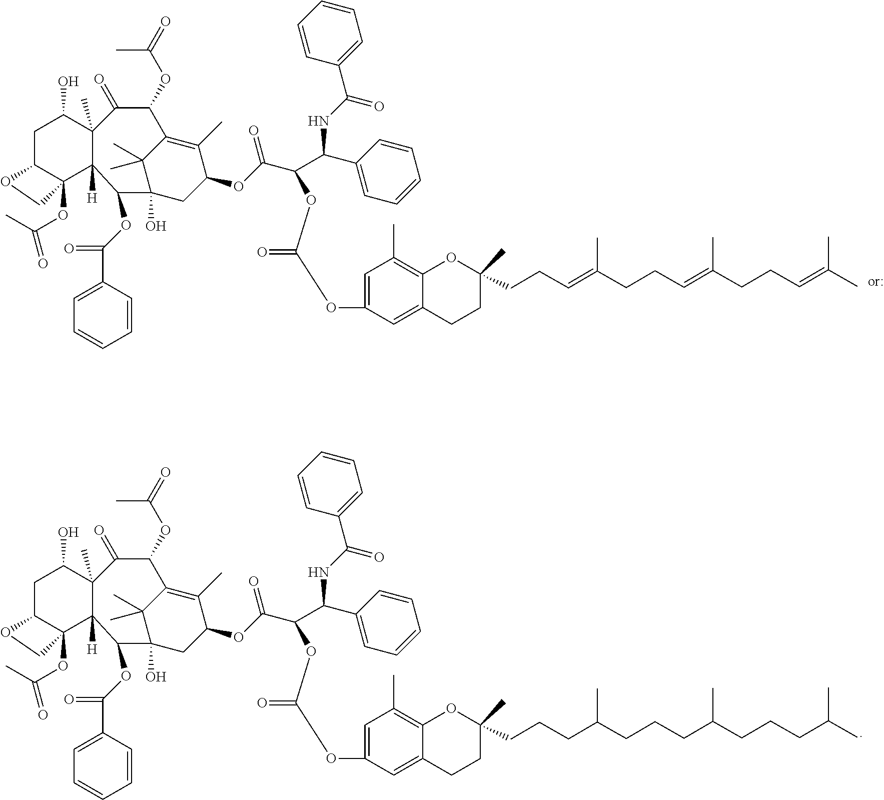 [SMILES: CC(=O)O[C@H]1C(=O)[C@@]2(C)[C@H]([C@H](OC(=O)c3ccccc3)[C@]3(O)C[C@H](OC(=O)[C@H](OC(=O)Oc4cc(C)c5c(c4)CC[C@@](C)(CC/C=C(\C)CC/C=C(\C)CCC=C(C)C)O5)[C@@H](NC(=O)c4ccccc4)c4ccccc4)C(C)=C1C3(C)C)[C@]1(OC(C)=O)CO[C@@H]1C[C@@H]2O ; CC(=O)O[C@H]1C(=O)[C@@]2(C)[C@H]([C@H](OC(=O)c3ccccc3)[C@]3(O)C[C@H](OC(=O)[C@H](OC(=O)Oc4cc(C)c5c(c4)CC[C@@](C)(CCCC(C)CCCC(C)CCCC(C)C)O5)[C@@H](NC(=O)c4ccccc4)c4ccccc4)C(C)=C1C3(C)C)[C@]1(OC(C)=O)CO[C@@H]1C[C@@H]2O]

Embodiment 89: A pharmaceutical composition comprising a pharmaceutically acceptable excipient and an effective amount of a composition comprising C-m-HDLs compositions of any one of Embodiments 15 through 20.

Embodiment 90: A pharmaceutical composition comprising a pharmaceutically acceptable excipient and an effective amount of a population of micellular nanoparticles which comprises:
a) water;
b) a disaccharide;
c) a population of C-m-HDLs wherein said nanoparticles in said population comprise:
   i) an amphiphilic, alpha-helical peptide that comprises an amino acid sequence of any one or more of SEQ ID NO: 1 through SEQ ID NO:36 wherein said peptide forms a HDL mimetic;
   ii) one or both of sphingomyelin and phosphatidyl choline, and optionally additional lipid(s) or phospholipid(s); and
   ii) a conjugate comprising an anchor moiety molecule and a paclitaxel that is releasably attached to each other through a cleavable bond;
wherein the C-m-HDLs in the population comprise a hydrophilic exterior surface and a hydrophobic core; and
further wherein the C-m-HDLs in the population have an average particle diameter of from about 11.5 nanometers to about 14 nanometers.

Embodiment 91: A pharmaceutical composition comprising an aqueous composition suitable for intravenous injection which composition comprise sterile water, sucrose and an effective amount of a population of C-m-HDLs which itself comprises: (a) an amphiphilic, alpha-helical peptide that comprises an amino acid sequence of SEQ ID NO:25, SEQ ID NO:28, SEQ ID NO:34, SEQ ID NO:35, or SEQ ID NO:36, (b) one or both of sphingomyelin and phosphatidyl choline, and optionally additional lipid(s) including phospholipid(s); and (c) a conjugate comprising paclitaxel and an anchor moiety, wherein the anchor moiety is releasably attached to paclitaxel through a carbonate bond; wherein said C-m-HDLs comprise: a hydrophilic exterior surface; a hydrophobic core comprising the conjugate; wherein said population of micellar nanoparticles has an average particle diameter of about 11.5 to about 14 nanometers, as measured by dynamic light scattering.

Embodiment 92: The pharmaceutical composition of Embodiment 91 wherein the population of C-m-HDLs has an average particle diameter of about 12.0 to about 13.5 nanometers, as measured by dynamic light scattering.

Embodiment 93: The pharmaceutical composition of Embodiment 92, wherein at least about 65% of the C-m-HDLs are within plus/minus about 2 nanometers of average diameter.

Embodiment 94: The pharmaceutical composition of Embodiment 92, wherein at least about 70% of the C-m-HDLs are within plus/minus about 2 nanometers of the average diameter.

Embodiment 95: A population of micellar nanoparticles comprising: (a) a peptide that forms a mimetic ligand to the SR-BI binding site of high density lipoprotein (HDL) on tumor cells; (b) a lipid; (c) a conjugate comprising a chemotherapeutic drug and an anchor moiety molecule, wherein the anchor moiety molecule is releasably attached to a chemotherapeutic drug; and a disaccharide, wherein the micellar nanoparticle comprises: a hydrophilic exterior surface; a hydrophobic core comprising the conjugate; wherein the micellar nanoparticle has a mean average particle diameter of about 12-about 13.5 nanometers.

Embodiment 96: The population of Embodiment 95, wherein the releasable attachment is via an ester, thioester, carbonate, thiocarbonate, carbamate, or a thiocarbamate bond.

Embodiment 97: The population of Embodiment 95 or 96 which further comprises a lipid molecule that is absorbable by SR-BI.

Embodiment 98: The population of Embodiment 95, 96 or 97, wherein the peptide is an amphiphilic, alpha-helical peptide.

Embodiment 99: The population of Embodiment 95, 96 or 97 wherein the peptide is any one of SEQ ID NOs: 25, 28, 34, 35 or 36 or combinations thereof.

Embodiment 100: The population of Embodiment 99 wherein the peptide comprises an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence set forth in any one of SEQ ID NOs: 25, 28, 34, 35 or 36.

Embodiment 101: A population of micellar nanoparticles which comprises:
(a) a peptide; (b) a lipid; (c) a conjugate comprising a chemotherapeutic drug and an anchor moiety molecule, wherein the anchor moiety molecule is releasably attached to a chemotherapeutic drug, wherein the attachment is via an ester, thioester, carbonate, thiocarbonate, carbamate, or a thiocarbamate bond; and (d) a disaccharide; wherein the micellar nanoparticle comprises: a hydrophilic exterior surface; a hydrophobic core comprising the conjugate; wherein the micellar nanoparticle has a mean average particle diameter of about 12-13.5 nanometers; wherein the peptide consists of an amino acid sequence as set forth in SEQ ID NOs: 25, 28, 34, 35 or 36.

Embodiment 102: The population of Embodiment 101, wherein the disaccharide is selected from sucrose, lactose, maltose, trehalose, cellobiose or lactulose.

Embodiment 103: The population of Embodiment 102, wherein the disaccharide is sucrose.

Embodiment 104: A population of micellar nanoparticle which comprises:
(a) an amphiphilic, alpha-helical peptide that comprises an amino acid sequence of SEQ ID NO: 25, SEQ ID NO:28, SEQ ID NO:34, SEQ ID NO:35 or SEQ ID NO:36; (b) sphingomyelin and optionally phosphatidyl choline and/or additional lipid(s) including phospholipid(s); (c) a conjugate comprising a chemotherapeutic drug and an anchor moiety molecule, wherein the anchor moiety molecule is releasably attached to a chemotherapeutic drug, through an ester, thioester, carbonate, thiocarbonate, carbamate, or a thiocarbamate bond; and (d) a disaccharide selected from sucrose, lactose, maltose, trehalose, cellobiose and lactulose; wherein said micellar nanoparticle comprises: a hydrophilic exterior surface; a hydrophobic core comprising the conjugate; wherein each of the micellar nanoparticles in the population of micellar nanoparticles has a mean average particle diameter of about 12-13.5 nanometers, as measured by dynamic light scattering.

Embodiment 105: The population of micellular nanoparticles of Embodiment 104, wherein at least about 65% of the nanoparticles are within plus/minus about 2 nanometers of the mean number diameter.

Embodiment 106: The population of micellular nanoparticles of Embodiment 104, wherein at least about 70% of the nanoparticles are within plus/minus about 2 nanometers of the mean number diameter.

Embodiment 107: The population of micellular nanoparticles of Embodiment 104, wherein at least about 75% of the nanoparticles are within plus/minus about 2 nanometers of the mean number diameter.

Embodiment 108: The population of micellular nanoparticles of Embodiment 104, wherein at least about 80% of the nanoparticles are within plus/minus about 2 nanometers of the mean number diameter.

Embodiment 109: A composition comprising population of micellular nanoparticles of any one of Embodiments 95 through 108, wherein said population is maintained in an aqueous environment.

Embodiment 110: A composition comprising a population of micellular nanoparticles of any one of Embodiments 95 through 109, wherein said composition is a lyophilized.

Embodiment 111: A composition comprising a population of lyophilized micellar nanoparticles according to Embodiment 110 wherein said disaccharide is sucrose.

Embodiment 112: The composition of Embodiment 111, wherein the concentration of sucrose in said nanoparticles ranges from about 20 to about 300 weight percent based on the weight of the nanoparticles.

Embodiment 113: The composition of Embodiment 111, wherein the concentration of sucrose in said nanoparticles ranges from about 50 to about 150 weight percent based on the weight of the nanoparticles.

Embodiment 114: The composition of Embodiment 111, wherein the concentration of sucrose in said nanoparticles ranges from about 75 to about 125 weight percent based on the weight of the nanoparticles.

Embodiment 115: The population of micellular nanoparticles according to any one of Embodiments 95-108 wherein the chemotherapeutic agent is selected from a hydroxy camptothecin (e.g., 7-ethyl-hydroxy camptothesin), doxorubicin, troxacitabine, vincristine, sirolimus, tubulysin A, docetaxel, paclitaxel, doxorubicin, daunorubicin, gemcitabine, cytarabine, troxacitabine, mertansine, or exatecan.

Embodiment 116: The population of micellular nanoparticles according to any one of Embodiment 115, wherein the lipid is cholesterol, cholecalciferol, β-, γ-, and 8-tocotrienol isoforms, β-, γ-, δ-tocopherol isoforms or combinations thereof.

Embodiment 117: The composition according to any one of Embodiments 109-114 wherein the chemotherapeutic agent is selected from AZD2811, a hydroxy camptothecin (e.g., 7-ethyl-hydroxy camptothesin), doxorubicin, troxacitabine, vincristine, sirolimus, tubulysin A, docetaxel, paclitaxel, doxorubicin, daunorubicin, gemcitabine, cytarabine, troxacitabine, mertansine, defosbarasertib, or exatecan.

Embodiment 118: The population of micellular nanoparticles according to any one of Embodiment 117, wherein the lipid is cholesterol, cholecalciferol, β-, γ-, and 8-tocotrienol isoforms, β-, γ-, δ-tocopherol isoforms or combinations thereof.

Embodiment 119: A library of conjugates as provided in Tables 1 through Table 5 at pages 6 through 18 supra.

Embodiment 120: A method for preparing a solution of a fully dissolved amphiphilic, alpha-helical peptide selected from an amino acid sequence as provided by SEQ ID NO: 25, SEQ ID NO: 28, SEQ ID NO: 34, SEQ ID NO: 35, SEQ ID NO:36, and combinations thereof in an aqueous-ethanol co-solvent, which method comprises combining in any order:
a) from about 50 to about 90 weight percent ethanol and from about 10 to about 50 weight percent water;
b) adding from about 2 to about 10 weight percent of an acid based on the weight of a) above;
c) adding from about 1 to about 2.5 weight percent of an amphiphilic, alpha-helical peptide selected from an amino acid sequence as provided by SEQ ID NO: 25, SEQ ID NO: 28, SEQ ID NO: 34, SEQ ID NO: 35, and SEQ ID NO:36 based on the total weight of the solvent solution of b) above; and stirring until a clear solution is obtained.

Embodiment 121: A method for preparing a soluble composition comprising micellar nanoparticles which method comprises: preparing a composition designated as “Composition 1”:
a) by combining in any order:
   i) ethanol and water to form a cosolvent having from about 50 to about 90 weight percent ethanol and from about 10 to about 50 weight percent water;
   ii) about 2 to 10 weight percent of an acid based on the weight of the cosolvent;
   iii) about 1 to 2.5 weight percent of a peptide of SEQ ID NO. 25, SEQ ID NO: 28, SEQ ID NO: 34, SEQ ID NO: 35, or SEQ ID NO: 36 based on the weight of the cosolvent of ii);
   wherein the total amount of i), ii) and iii) equals 100%, and stirring the composition until the solution is clear evidencing that the peptide is completely dissolved;
b) combine the solution generated in of a) with from about 3 to 6 weight percent of a lipid compositions based on the weight of the solution of b) wherein said lipid composition comprises:
   iv) about 50 to 70 weight percent of an additional lipid component such as 1-palmitoyl-2-oleoyl-phosphatidylcholine (POPC) (commercially available from Lipoid, Newark, New Jersey, USA)
   v) about 18 to about 30 weight percent of sphingomyelin (SM) (commercially available from Lipoid, Newark, New Jersey, USA), and
   vi) about 12 to about 35 weight percent of a conjugate as described above; and further wherein the total of the solution in a) and the lipid composition in b) equals 100%, and stirring as necessary to provide for a clear solution and designating the clear solution as “Composition 1.”

Embodiment 122: A method for reducing the size of micellar nanoparticles which method comprises:
combining water with from about 2 to 6 weight percent of a disaccharide and mix until homogeneous and designated that solution as “Composition 2”;
adding Composition 2 to a first loading chamber of a three-chamber mixing device which comprises two separate loading chambers and a single reaction chamber;
adding an appropriate amount of a Composition 1 prepared as above into the other loading chamber of said two loading chambers in the device;
chaotically mixing the two compositions into the reaction chamber under a controlled flow rate ratio of from about 4:1 to about 10:1 of Composition 2 to Composition 1 at a temperature of from about 30° to about 70° C. while maintaining the flow from each chamber until mixing is complete while maintaining a temperature of from about 30° to about 70° C.;
thereby providing a nanoparticle suspension having a mean number average diameter for the nanoparticles of about 11.5 to about 14 nanometers, and preferably about 12 nm to about 13.5 nanometers.

Embodiment 123: The method of Embodiments 121 or 122, wherein the micellular nanoparticles have a distribution of particle sizes wherein at least about 65% of said particles are within +/−3 nm.

Embodiment 124: The method of Embodiments 121 or 122, wherein the micellular nanoparticles have a distribution of particle sizes wherein at least about 70% of said particles are within +/−3 nm.

Embodiment 125: The method of Embodiments 121 or 122, wherein the micellular nanoparticles have a distribution of particle sizes wherein at least about 75% of said particles are within +/−3 nm.

Embodiment 126: The method of Embodiments 121 or 122 wherein the micellular nanoparticles have a distribution of particle sizes wherein at least about 80% of said particles are within +/−3 nm.

Embodiment 127: The method of Embodiments 121 or 122 which further comprises removing at least a portion of the ethanol and acetic acid by either dialysis or tangential flow filtration (TFF).

Embodiment 128: The method of Embodiment 127 wherein the resulting suspension is sterile filtered preferably using a 0.2 micron filter.

Embodiment 129: The method of Embodiment 128 which further comprises lyophilizing the composition.

Embodiment 130: The method of Embodiment 122 wherein the disaccharide is selected from sucrose, lactose, maltose, trehalose, cellobiose and lactulose.

Embodiment 131: The method of Embodiment 130, wherein the disaccharide is sucrose.

Embodiment 132: The method of Embodiment 131, wherein the sucrose is used in an aqueous solution during manufacture of the micellular nanoparticles in an amount of about 2, or about 3, or about 4, or about 5 or about 6 weight percent sucrose based on the weight of the aqueous solution.

Embodiment 133: The method of Embodiment 132, wherein the sucrose is used in an aqueous solution during manufacture of the micellular nanoparticles in an amount of about 5 weight percent sucrose based on the weight of the aqueous solution.

Embodiment 134: The method of Embodiment 122, wherein the mean number average diameter size of the micellular nanoparticles after synthesis is reduced by about 10% as compared to the diameter of the micellular nanoparticles made in the same manner but without sucrose.

Embodiment 135: The method of Embodiment 134, wherein the mean number average diameter size of the micellular nanoparticles after synthesis is reduced by about 15% as compared to the diameter of the micellular nanoparticles made in the same manner but without sucrose.

Embodiment 136: The method of Embodiment 134, wherein the mean number average diameter size of the micellular nanoparticles after synthesis is reduced by about 20% as compared to the diameter of the micellular nanoparticles made in the same manner but without sucrose.

Embodiment 137: The method of Embodiment 134, wherein the mean number average diameter size of the micellular nanoparticles after synthesis is reduced by about 22% as compared to the diameter of the micellular nanoparticles made in the same manner but without sucrose.

Embodiment 138: A method for treating a cancer in a patient which method comprises a) confirming that the cancer cells overexpress SR-BI; and b) administering to said patient an effective amount of a population of nanoparticles as described herein and which comprise a peptide aggregate that mimics the binding site of high density lipoprotein (HDL) to SR-BI as well as a conjugate having a chemotherapeutic agent and an anchor moiety bound together by a cleavable bond.

Embodiment 139: The method of Embodiment 138 wherein said nanoparticles are administered directly or as part of a pharmaceutical composition.

Embodiment 140: The method of Embodiment 139 wherein upon binding of said nanoparticles to SR-BI on said cancer cells, the tumor cell absorbs the conjugate via the anchor moiety.

Embodiment 141: The method of Embodiment 140 wherein during absorption, intracellular enzymes cleave the cleavable bond thereby releasing the chemotherapeutic agent.

Embodiment 142: The method of Embodiment 138 wherein said cancer is a solid mass tumor.

Embodiment 143: The method of Embodiment 138 wherein said cancer is a blood borne tumor.

Embodiment 144: The population or composition of any of the Embodiments described herein, wherein said population or composition is formulated in a reconstituted aqueous composition suitable for intravenous injection.

Embodiment 145: A pharmaceutical composition comprising a reconstituted aqueous composition suitable for intravenous injection which composition comprise an effective amount of a micellular nanoparticle which itself comprises: (a) an amphiphilic, alpha-helical peptide that comprises an amino acid sequence of SEQ ID NO:25, SEQ ID NO:28, SEQ ID NO: 34, SEQ ID NO:35, or SEQ ID NO:36, (b) one or both of sphingomyelin and phosphatidyl choline and/or additional lipid(s) including phospholipid(s); (c) a conjugate comprising a chemotherapeutic drug and an anchor moiety molecule, wherein the anchor moiety molecule is releasably attached to the chemotherapeutic drug through an ester, thioester, carbonate, thiocarbonate, carbamate, or a thiocarbamate bond; and (d) a disaccharide selected from sucrose, lactose, maltose, trehalose, cellobiose and lactulose; wherein said micellar nanoparticle comprises: a hydrophilic exterior surface; a hydrophobic core comprising the conjugate; wherein each of the micellar nanoparticles in the population of micellar nanoparticles has a mean average particle diameter of about 12-13.5 nanometers, as measured by dynamic light scattering.

Embodiment 146: A kit of parts comprising a first vial comprising a unit dose of a lyophilized composition as described in any of the Embodiments set forth herein and a second vial comprising a unit amount of a sterile aqueous composition, and optionally a needle syringe.

Embodiment 147: A kit of parts of Embodiment 146, wherein said kit further comprises a product insert with instruction for use.

Embodiment 148: A conjugate of mertansine-ò-tocopherol thiocarbonate

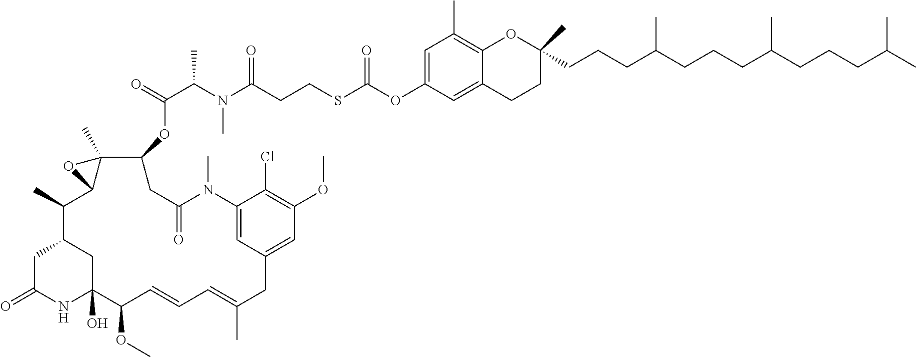 [SMILES: COc1cc2cc(c1Cl)N(C)C(=O)C[C@H](OC(=O)[C@H](C)N(C)C(=O)CCSC(=O)Oc1cc(C)c3c(c1)CC[C@@](C)(CCCC(C)CCCC(C)CCCC(C)C)O3)[C@]1(C)O[C@H]1[C@H](C)[C@H]1CC(=O)N[C@](O)(C1)[C@H](OC)/C=C/C=C(\C)C2]

Embodiment 149: A conjugate of Exatecan-ò-tocopherol carbamate

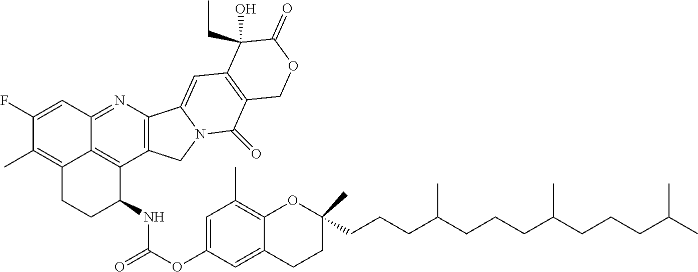 [SMILES: CC[C@@]1(O)C(=O)OCc2c1cc1n(c2=O)Cc2c-1nc1cc(F)c(C)c3c1c2[C@@H](NC(=O)Oc1cc(C)c2c(c1)CC[C@@](C)(CCCC(C)CCCC(C)CCCC(C)C)O2)CC3]

Embodiment 150: A conjugate of Gemcitibine-o-tocopherol carbamate

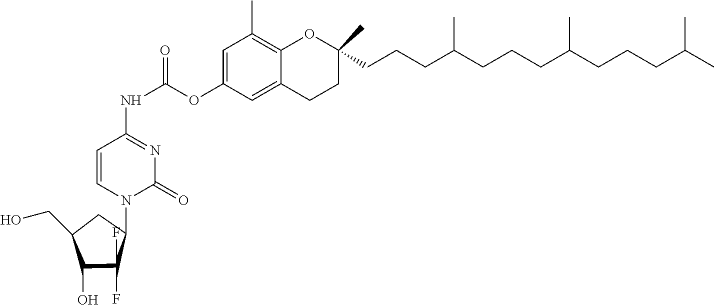

Embodiment 151: A conjugate of Gemcitibine-ò-tocopherol carbonate

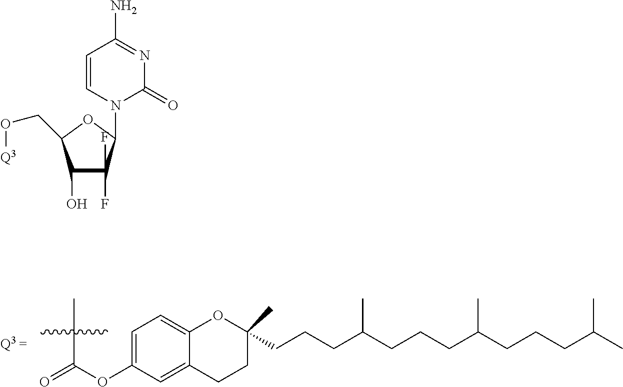

where the squiggle is the point of attachment of the Q3 to gemcitabine.

Embodiment 152: A conjugate of Eribuline-δ-tocopherol carbamate

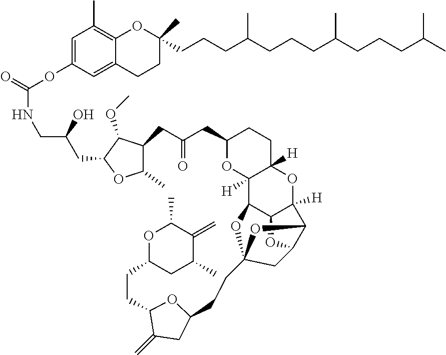 [SMILES: C=C1C[C@H](CC[C@@]23C[C@H]4O[C@H]5[C@@H](O2)[C@H]2O[C@@H](CC(=O)C[C@@H]6[C@@H](OC)[C@@H](C[C@H](O)CNC(=O)Oc7cc(C)c8c(c7)CC[C@@](C)(CCCC(C)CCCC(C)CCCC(C)C)O8)O[C@H]6C)CC[C@@H]2O[C@H]5[C@H]4O3)O[C@H]1CC[C@H]1C[C@@H](C)C(=C)[C@@H](C)O1]

Embodiment 153: A conjugate of mertansine-ò-tocotrienol thiocarbonate

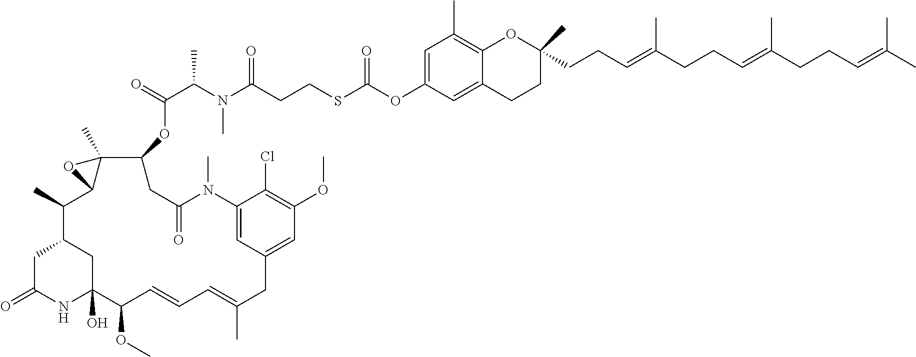 [SMILES: COc1cc2cc(c1Cl)N(C)C(=O)C[C@H](OC(=O)[C@H](C)N(C)C(=O)CCSC(=O)Oc1cc(C)c3c(c1)CC[C@@](C)(CC/C=C(\C)CC/C=C(\C)CCC=C(C)C)O3)[C@]1(C)O[C@H]1[C@H](C)[C@H]1CC(=O)N[C@](O)(C1)[C@H](OC)/C=C/C=C(\C)C2]

Embodiment 154: A conjugate of Exatecan-δ-tocotrienol carbamate

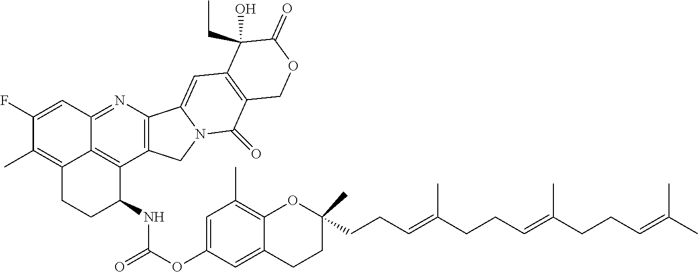 [SMILES: CC[C@@]1(O)C(=O)OCc2c1cc1n(c2=O)Cc2c-1nc1cc(F)c(C)c3c1c2[C@@H](NC(=O)Oc1cc(C)c2c(c1)CC[C@@](C)(CC/C=C(\C)CC/C=C(\C)CCC=C(C)C)O2)CC3]

Embodiment 155: A conjugate of Gemcitibine-δ-tocotrienol carbamate

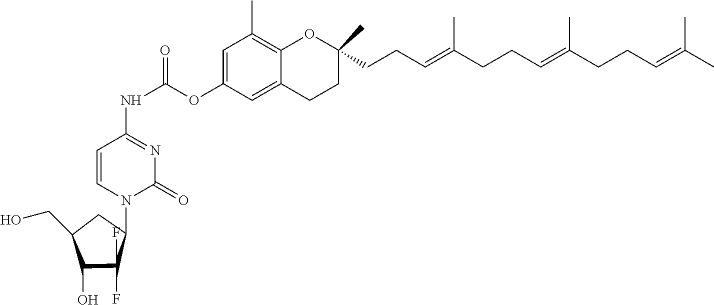

Embodiment 156: A conjugate of Gemcitibine-δ-tocotrienol carbonate

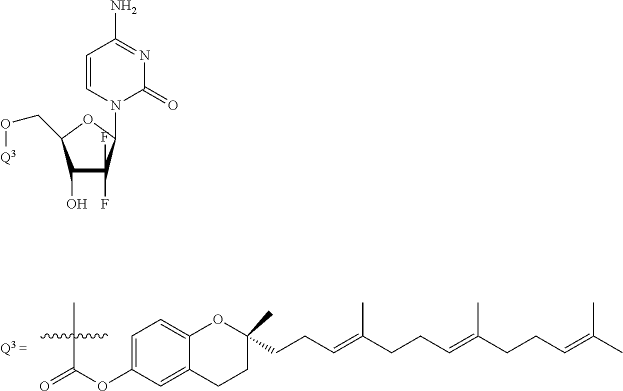

where the squiggle is the point of attachment of the Q3 to gemcitabine.

Embodiment 157: A conjugate of uribuline-δ-tocotrienol carbamate

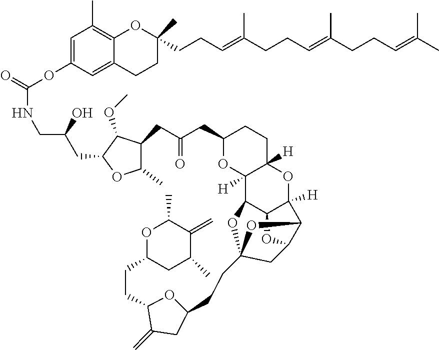 [SMILES: C=C1C[C@H](CC[C@@]23C[C@H]4O[C@H]5[C@@H](O2)[C@H]2O[C@@H](CC(=O)C[C@@H]6[C@@H](OC)[C@@H](C[C@H](O)CNC(=O)Oc7cc(C)c8c(c7)CC[C@@](C)(CC/C=C(\C)CC/C=C(\C)CCC=C(C)C)O8)O[C@H]6C)CC[C@@H]2O[C@H]5[C@H]4O3)O[C@H]1CC[C@H]1C[C@@H](C)C(=C)[C@@H](C)O1]

Embodiment 158: A conjugate of Uribuline-cholesterol

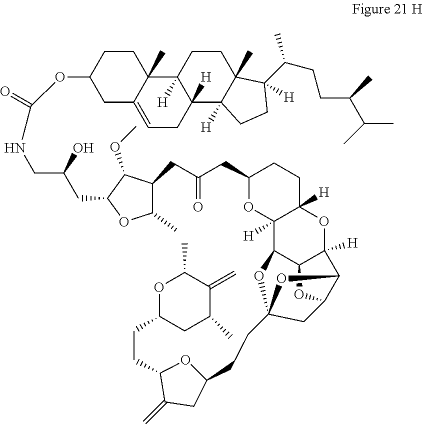

Embodiment 159: A conjugate of gemcitibine-cholesterol (carbamate linker)

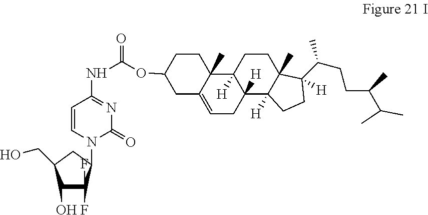

Embodiment 160: A conjugate of Exatecan-cholesterol

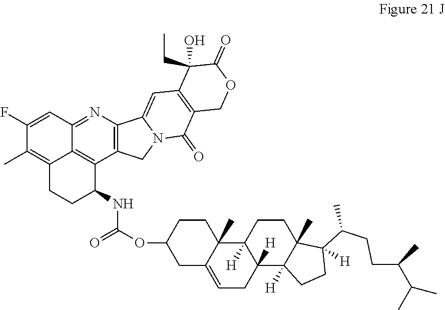

Embodiment 161: A conjugated of mertansine-cholesterol

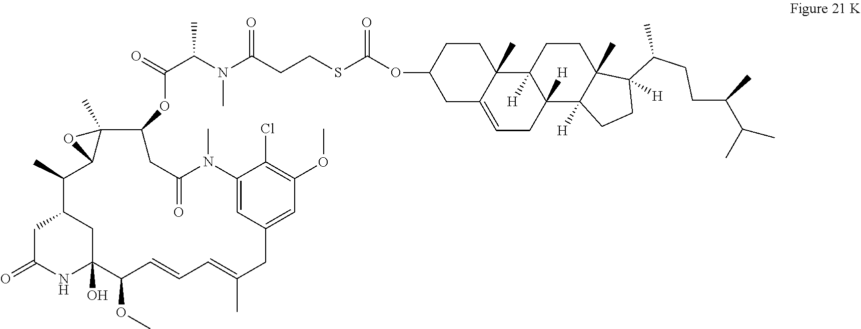

Embodiment 162: A conjugate of gemcitibine-cholesterol (carbonate linker)

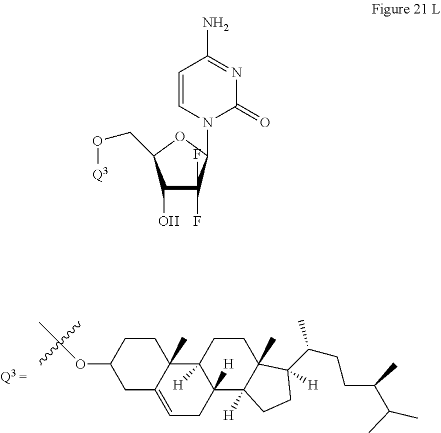

Embodiment 163: A conjugate of mertansine with an anchor moiety selected from coprostanol, β-sitosterol, sitostanol, stigmasterol, stigmastanol, campesterol, brassicasterol, ergosterol, retinol, cholecalciferol and ergocalciferol.

Embodiment 164: A conjugate of gemcitabine with an anchor moiety selected from coprostanol, β-sitosterol, sitostanol, stigmasterol, stigmastanol, campesterol, brassicasterol, ergosterol, retinol, cholecalciferol and ergocalciferol.

Embodiment 165: A conjugate of uribuline with an anchor moiety selected from coprostanol, β-sitosterol, sitostanol, stigmasterol, stigmastanol, campesterol, brassicasterol, ergosterol, retinol, cholecalciferol and ergocalciferol.

Embodiment 166: A conjugate of exatecan with an anchor moiety selected from coprostanol, β-sitosterol, sitostanol, stigmasterol, stigmastanol, campesterol, brassicasterol, ergosterol, retinol, cholecalciferol and ergocalciferol.

Embodiment 167: In some embodiments, doxorubicin is not conjugated to an anchor molecule for use herein. In some embodiments, neither alpha-tocotrienol nor alpha-tocopherol are used as the anchor molecule except for thiocarbonate cleavable bonds.

Embodiment 168: a conjugate of paclitaxel such as any of the conjugates shown in Table 6 above.

All references, patents, and patent applications cited herein are incorporated into this application in their entirety.

What is claimed is:

1. An aqueous composition comprising a population of HDL mimetic micellular nanoparticles (C-m-HDLs) which composition comprises:
a) water;
b) a disaccharide;
c) a population of C-m-HDLs wherein said C-m-HDLs in said population comprise:
i) an amphiphilic, alpha-helical peptide or protein wherein said peptide or protein is an HDL mimetic structure on said C-m-HDLs;
ii) one or both of sphingomyelin and phosphatidyl choline, and optionally additional lipid(s) or phospholipid(s); and
iii) a conjugate comprising an anchor moiety molecule and a chemotherapeutic agent that is releasably attached to each other through a cleavable bond;
wherein the C-m-HDLs in the population comprise a hydrophilic exterior surface and a hydrophobic core; and
further wherein the C-m-HDLs in the population have an average particle diameter of from about 11.5 nanometers to about 14 nanometers as measured by dynamic light scattering.

2. An aqueous composition comprising a population of C-m-HDLs which composition comprises:
a) water;
b) a disaccharide;
c) a population of C-m-HDLs which population comprises:
i) an amphiphilic, alpha-helical peptide that comprises an amino acid sequence of any one or more of SEQ ID NO:1 through SEQ ID NO:36;
ii) one or both of sphingomyelin and phosphatidyl choline, and optionally additional lipid(s) or phospholipid(s); and
iii) a conjugate comprising an anchor moiety molecule and a chemotherapeutic agent that are releasably attached to each other through a cleavable bond;
wherein the C-m-HDLs in the population comprise a hydrophilic exterior surface and a hydrophobic core; and
further wherein the C-m-HDLs in the population have an average particle diameter of from about 11.5 nanometers to about 14 nanometers as measured by dynamic light scattering.

3. The aqueous composition of claim 2, wherein said C-m-HDLs in said population have an average particle diameter of from about 12 nanometers to about 13.5 nanometers.

4. The aqueous composition of claim 3, wherein said disaccharide is selected from sucrose, lactose, maltose, trehalose, cellobiose and lactulose.

5. The aqueous composition of claim 4, wherein said disaccharide is sucrose.

6. A lyophilized composition of the composition of claim 1.

7. A lyophilized composition of the composition of claim 2.

8. A lyophilized composition of the composition of claim 3.

9. A population of C-m-HDLs comprising:
(a) an amphiphilic, alpha-helical peptide that comprises an amino acid sequence of any one of SEQ ID NO:25, SEQ ID NO:28, SEQ ID NO:34, SEQ ID NO:35, SEQ ID NO. 36, or combinations thereof wherein said peptide forms an HDL mimetic structure on said nanoparticle;
(b) one or both of sphingomyelin and phosphatidyl choline, and optionally additional lipid(s) or phospholipid(s);
(c) a conjugate comprising an anchor moiety molecule and a chemotherapeutic agent that is releasably attached to each other through a cleavable bond; and
wherein the micellular nanoparticles in the population have a hydrophilic exterior surface and a hydrophobic core; and
further wherein the micellular nanoparticles in the population have an average particle diameter of from about 11.5 to about 14.0 nanometers, as measured by dynamic light scattering.

10. The population of C-m-HDLs of claim 9, wherein at least about 70% of said C-m-HDLs in said population are within plus/minus about 3 nanometers of the average particle diameter.

11. The population of C-m-HDLs of claim 9, wherein the releasable bond is selected from an ester, a thioester, a carbonate, a thiocarbonate, a carbamate, or a thiocarbamate bond.

12. The population of C-m-HDLs of claim 9, wherein the releasable bond is a carbonate bond.

13. The population of C-m-HDLs of claim 9, wherein said C-m-HDLs in the population have an average particle diameter of from about 12 to about 13.5 nanometers.

14. The population of C-m-HDLs of claim 13, wherein at least about 70% of said C-m-HDLs in said population are within plus/minus about 2 nanometers of the average particle diameter.

15. The population of C-m-HDLs of claim 13, wherein the paclitaxel-anchor moiety is selected from a conjugate of Table 1.

16. The population of C-m-HDLs of claim 13, wherein the anchor moiety is cholesterol or β-, γ-, and δ-tocotrienol or β-, γ-, and δ-tocopherol.

17. A population of C-m-HDLs which population comprises:
a) an amphiphilic, alpha-helical peptide that comprises an amino acid sequence of any one of SEQ ID NO:25, SEQ ID NO:28, SEQ ID NO:34, SEQ ID NO:35, SEQ ID NO. 36, or combinations thereof wherein said peptide forms an HDL mimetic structure on said C-m-HDLs;
(b) one or both of sphingomyelin and phosphatidyl choline, and optionally additional lipid(s) or phospholipid(s);
(c) a chemotherapeutic-lipid conjugate selected from conjugates in Tables 1-5:
TABLE 1

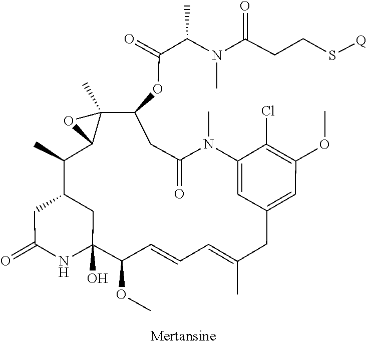

| No. | Q | Trivial Name |
|---|---|---|
| 1 | 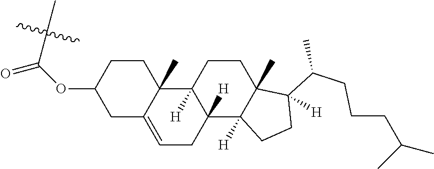 | Mertansine-cholesterol conjugate with a cleavable carbonate bond |
| 2 | 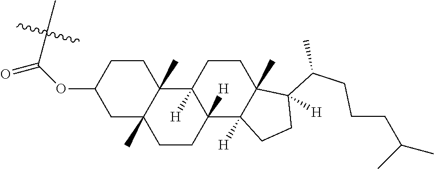 | Mertansine-coprostanol conjugate with a cleavable carbonate bond |
| 3 | 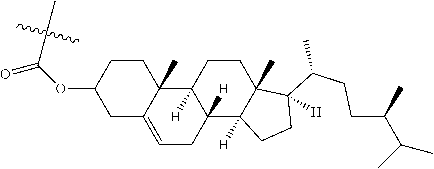 | Mertansine-campesterol conjugate with a cleavable carbonate bond |
| 4 | 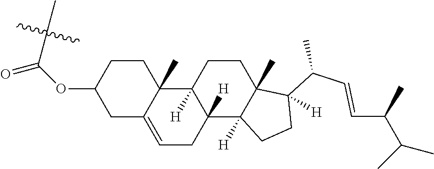 | Mertansine-brassicasterol conjugate with a cleavable carbonate bond |
| 5 | 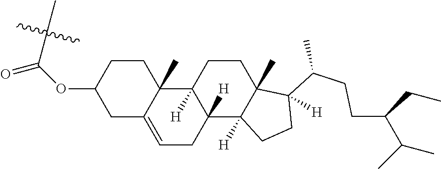 | Mertansine-sitosterol conjugate with a cleavable carbonate bond |
| 6 | 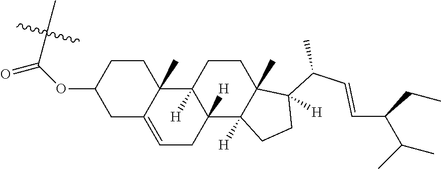 | Mertansine-stigmasterol conjugate with a cleavable carbonate bond |
| 7 | 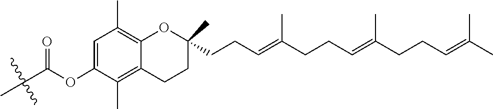 [SMILES: *C(=O)Oc1cc(C)c2c(c1C)CC[C@@](C)(CC/C=C(\C)CC/C=C(\C)CCC=C(C)C)O2] | Mertansine-β-tocotrienol conjugate with a cleavable carbonate bond |
| 8 | 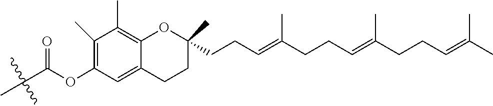 [SMILES: *C(=O)Oc1cc2c(c(C)c1C)O[C@](C)(CC/C=C(\C)CC/C=C(\C)CCC=C(C)C)CC2] | Mertansine-γ-tocotrienol conjugate with a cleavable carbonate bond |
| 9 | 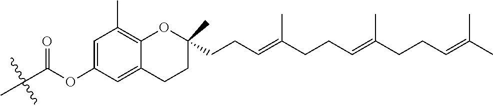 [SMILES: *C(=O)Oc1cc(C)c2c(c1)CC[C@@](C)(CC/C=C(\C)CC/C=C(\C)CCC=C(C)C)O2] | Mertansine-δ-tocotrienol conjugate with a cleavable carbonate bond |
| 10 | 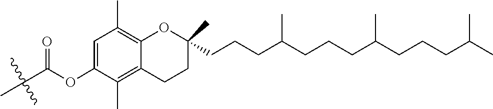 [SMILES: *C(=O)Oc1cc(C)c2c(c1C)CC[C@@](C)(CCCC(C)CCCC(C)CCCC(C)C)O2] | Mertansine-β-tocopherol conjugate with a cleavable carbonate bond |
| 11 | 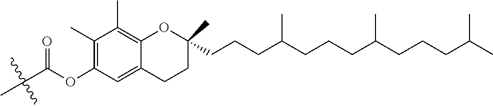 [SMILES: *C(=O)Oc1cc2c(c(C)c1C)O[C@](C)(CCCC(C)CCCC(C)CCCC(C)C)CC2] | Mertansine-γ-tocopherol conjugate with a cleavable carbonate bond |
| 12 | 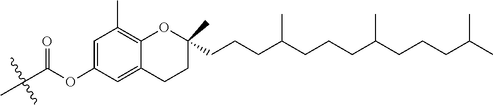 [SMILES: *C(=O)Oc1cc(C)c2c(c1)CC[C@@](C)(CCCC(C)CCCC(C)CCCC(C)C)O2] | Mertansine-δ-tocopherol conjugate with a cleavable carbonate bond |
| 13 | 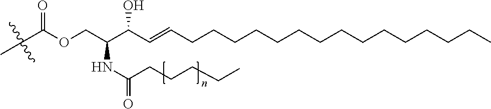 | n = 0 to 10 Mertansine-ceramide conjugate with a cleavable carbonate bond |
| 14 | 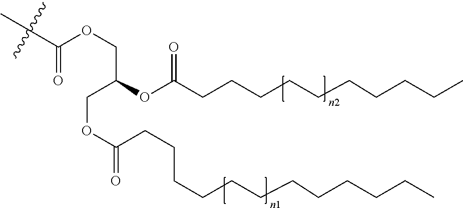 | n1 = 0 to 4 n2 = 0 to 4 Mertansine-diacyl- glycerol conjugate with a cleavable carbonate bond |

TABLE 2

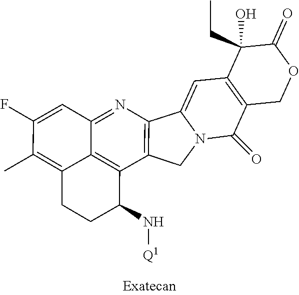

| No. | Q¹ | Trivial Name |
|---|---|---|
| 15 | 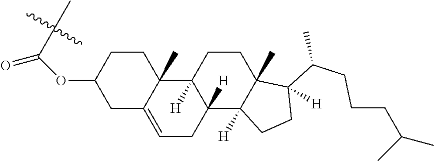 | Exatecan-cholesterol conjugate with a cleavable carbamate bond |
| 16 | 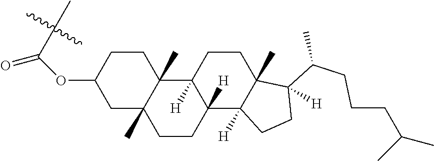 | Exatecan-coprostanol conjugate with a cleavable carbamate bond |
| 17 | 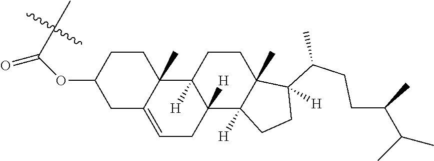 | Exatecan-campesterol conjugate with a cleavable carbamate bond |
| 18 | 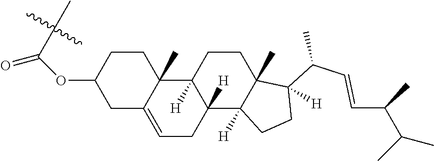 | Exatecan-brassicasterol conjugate with a cleavable carbamate bond |
| 19 | 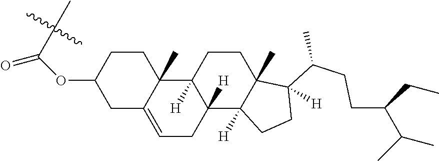 | Exatecan-sitosterol conjugate with a cleavable carbamate bond |
| 20 | 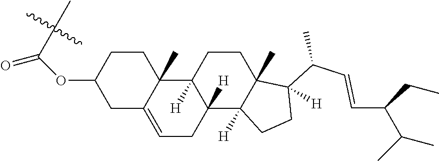 | Exatecan-stigmasterol conjugate with a cleavable carbamate bond |
| 21 | 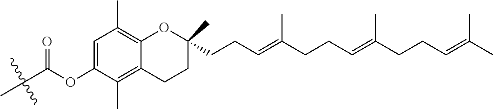 [SMILES: *C(=O)Oc1cc(C)c2c(c1C)CC[C@@](C)(CC/C=C(\C)CC/C=C(\C)CCC=C(C)C)O2] | Exatecan-β-tocotrienol conjugate with a cleavable carbamate bond |
| 22 | 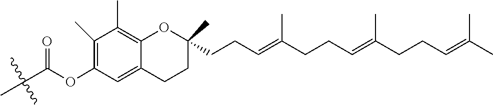 [SMILES: *C(=O)Oc1cc2c(c(C)c1C)O[C@](C)(CC/C=C(\C)CC/C=C(\C)CCC=C(C)C)CC2] | Exatecan-γ-tocotrienol conjugate with a cleavable carbamate bond |
| 23 | 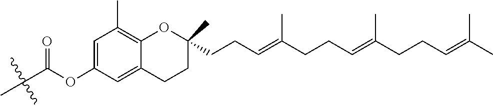 [SMILES: *C(=O)Oc1cc(C)c2c(c1)CC[C@@](C)(CC/C=C(\C)CC/C=C(\C)CCC=C(C)C)O2] | Exatecan-δ-tocotrienol conjugate with a cleavable carbamate bond |
| 24 | 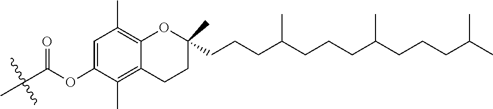 [SMILES: *C(=O)Oc1cc(C)c2c(c1C)CC[C@@](C)(CCCC(C)CCCC(C)CCCC(C)C)O2] | Exatecan-β-tocopherol conjugate with a cleavable carbamate bond |
| 25 | 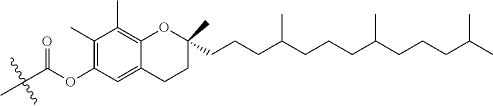 [SMILES: *C(=O)Oc1cc2c(c(C)c1C)O[C@](C)(CCCC(C)CCCC(C)CCCC(C)C)CC2] | Exatecan-γ-tocopherol conjugate with a cleavable carbamate bond |
| 26 | 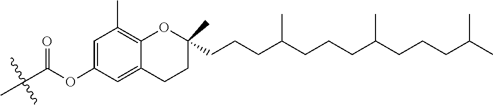 [SMILES: *C(=O)Oc1cc(C)c2c(c1)CC[C@@](C)(CCCC(C)CCCC(C)CCCC(C)C)O2] | Exatecan-δ-tocopherol conjugate with a cleavable carbamate bond |
| 27 | 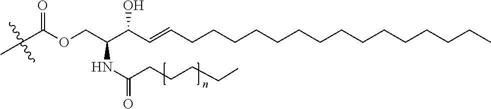 | n = 0 to 10 Exatecan-ceramide conjugate with a cleavable carbamate bond |
| 28 | 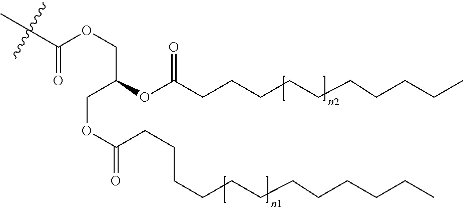 | n1 = 0 to 4 n2 = 0 to 4 Exatecan-diacylglycerol conjugate with a cleavable carbamate bond |

TABLE 3

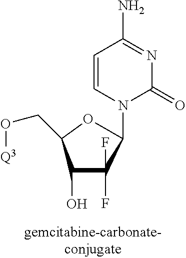

| No. | Q³ | Trivial Name |
|---|---|---|
| 29 | 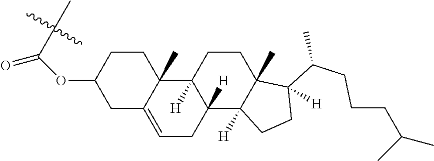 | Gemcitabine-cholesterol conjugate with a cleavable carbonate bond |
| 30 | 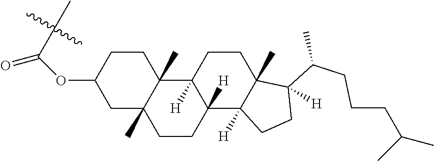 | Gemcitabine-coprostanol conjugate with a cleavable carbonate bond |
| 31 | 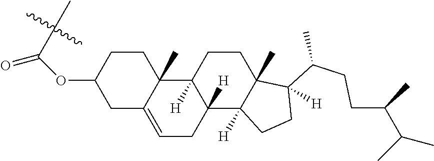 | Gemcitabine-campesterol conjugate with a cleavable carbonate bond |
| 32 | 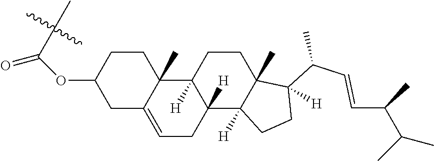 | Gemcitabine-brassicasterol conjugate with a cleavable carbonate bond |
| 33 | 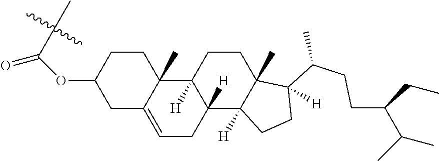 | Gemcitabine-sitosterol conjugate with a cleavable carbonate bond |
| 34 | 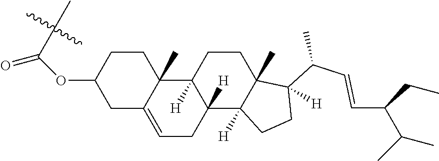 | Gemcitabine-stigmasterol conjugate with a cleavable carbonate bond |
| 35 | 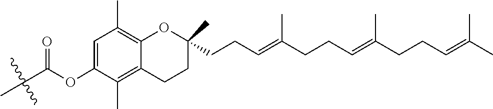 [SMILES: *C(=O)Oc1cc(C)c2c(c1C)CC[C@@](C)(CC/C=C(\C)CC/C=C(\C)CCC=C(C)C)O2] | Gemcitabine-β-tocotrienol conjugate with a cleavable carbonate bond |
| 36 | 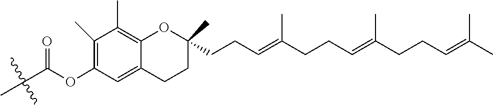 [SMILES: *C(=O)Oc1cc2c(c(C)c1C)O[C@](C)(CC/C=C(\C)CC/C=C(\C)CCC=C(C)C)CC2] | Gemcitabine-γ-tocotrienol conjugate with a cleavable carbonate bond |
| 37 | 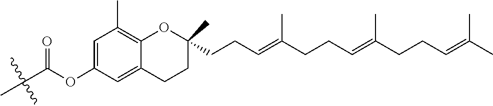 [SMILES: *C(=O)Oc1cc(C)c2c(c1)CC[C@@](C)(CC/C=C(\C)CC/C=C(\C)CCC=C(C)C)O2] | Gemcitabine-δ-tocotrienol conjugate with a cleavable carbonate bond |
| 38 | 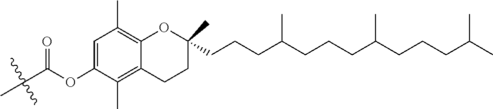 [SMILES: *C(=O)Oc1cc(C)c2c(c1C)CC[C@@](C)(CCCC(C)CCCC(C)CCCC(C)C)O2] | Gemcitabine-β-tocopherol conjugate with a cleavable carbonate bond |
| 39 | 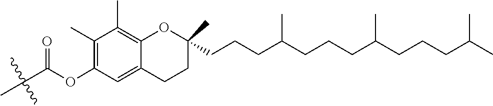 [SMILES: *C(=O)Oc1cc2c(c(C)c1C)O[C@](C)(CCCC(C)CCCC(C)CCCC(C)C)CC2] | Gemcitabine-γ-tocopherol conjugate with a cleavable carbonate bond |
| 40 | 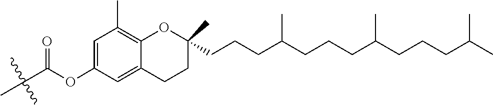 [SMILES: *C(=O)Oc1cc(C)c2c(c1)CC[C@@](C)(CCCC(C)CCCC(C)CCCC(C)C)O2] | Gemcitabine-δ-tocopherol conjugate with a cleavable carbonate bond |
| 41 | 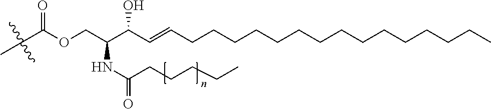 | n = 0 to 10 Gemcitabine-ceramide conjugate with a cleavable carbonate bond |
| 42 | 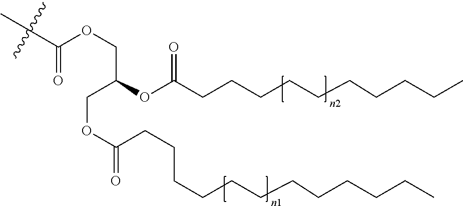 | n1 = 0 to 4 n2 = 0 to 4 Gemcitabine-diacyl- glycerol conjugate with a cleavable carbonate bond |

TABLE 4

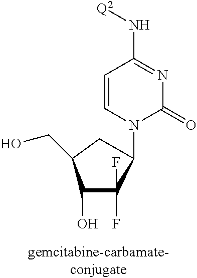

| No. | Q² | Trivial Name |
|---|---|---|
| 43 | 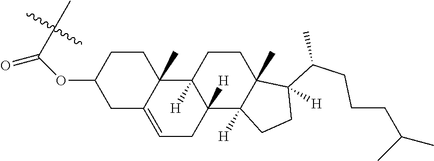 | Gemcitabine-cholesterol conjugate with a cleavable carbamate bond |
| 44 | 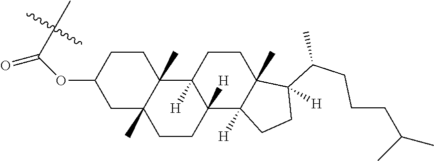 | Gemcitabine-coprostanol conjugate with a cleavable carbamate bond |
| 45 | 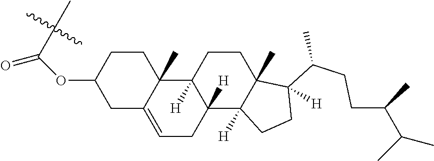 | Gemcitabine-campesterol conjugate with a cleavable carbamate bond |
| 46 | 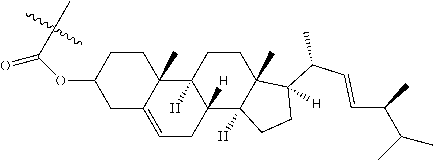 | Gemcitabine-brassicasterol conjugate with a cleavable carbamate bond |
| 47 | 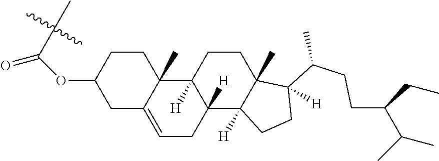 | Gemcitabine-sitosterol conjugate with a cleavable carbamate bond |
| 48 | 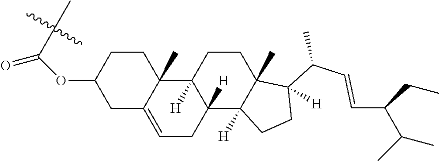 | Gemcitabine-stigmasterol conjugate with a cleavable carbamate bond |
| 49 | 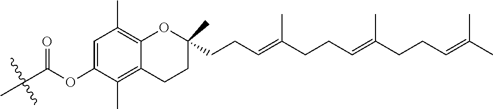 [SMILES: *C(=O)Oc1cc(C)c2c(c1C)CC[C@@](C)(CC/C=C(\C)CC/C=C(\C)CCC=C(C)C)O2] | Gemcitabine-β-tocotrienol conjugate with a cleavable carbamate bond |
| 50 | 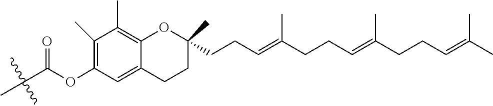 [SMILES: *C(=O)Oc1cc2c(c(C)c1C)O[C@](C)(CC/C=C(\C)CC/C=C(\C)CCC=C(C)C)CC2] | Gemcitabine-γ-tocotrienol conjugate with a cleavable carbamate bond |
| 51 | 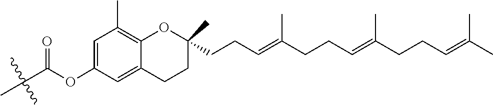 [SMILES: *C(=O)Oc1cc(C)c2c(c1)CC[C@@](C)(CC/C=C(\C)CC/C=C(\C)CCC=C(C)C)O2] | Gemcitabine-δ-tocotrienol conjugate with a cleavable carbamate bond |
| 52 | 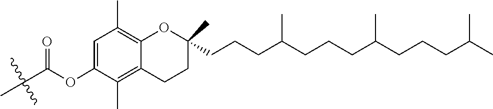 [SMILES: *C(=O)Oc1cc(C)c2c(c1C)CC[C@@](C)(CCCC(C)CCCC(C)CCCC(C)C)O2] | Gemcitabine-β-tocopherol conjugate with a cleavable carbamate bond |
| 53 | 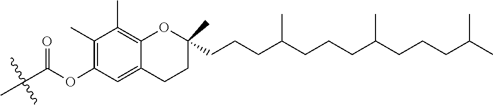 [SMILES: *C(=O)Oc1cc2c(c(C)c1C)O[C@](C)(CCCC(C)CCCC(C)CCCC(C)C)CC2] | Gemcitabine-γ-tocopherol conjugate with a cleavable carbamate bond |
| 54 | 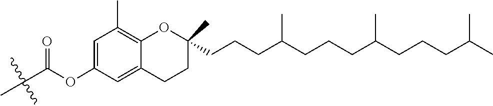 [SMILES: *C(=O)Oc1cc(C)c2c(c1)CC[C@@](C)(CCCC(C)CCCC(C)CCCC(C)C)O2] | Gemcitabine-δ-tocopherol conjugate with a cleavable carbamate bond |
| 55 | 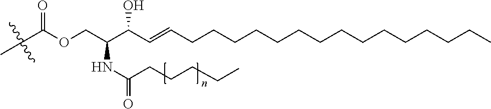 | n = 0 to 10 Gemcitabine-ceramide conjugate with a cleavable carbamate bond |
| 56 | 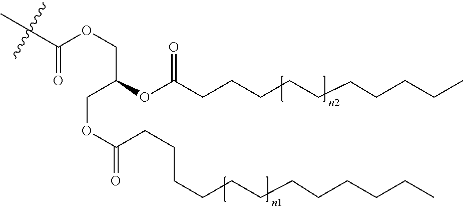 | n1 = 0 to 4 n2 = 0 to 4 Gemcitabine-diacylglycerol conjugate with a cleavable carbamate bond |

TABLE 5

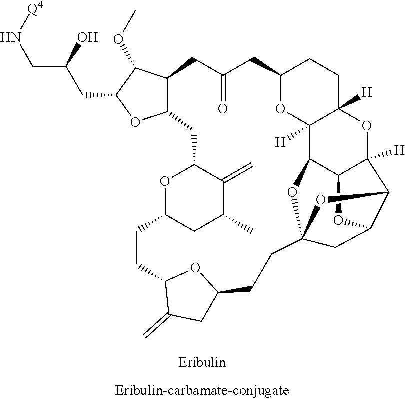

| No. | Q⁴ | Trivial Name |
|---|---|---|
| 57 | 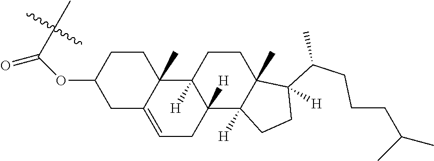 | Eribulin-cholesterol conjugate with a cleavable carbamate bond |
| 58 | 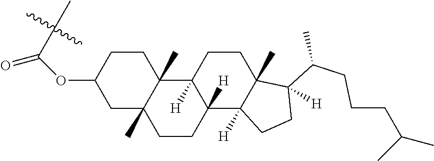 | Eribulin-coprostanol conjugate with a cleavable carbamate bond |
| 59 | 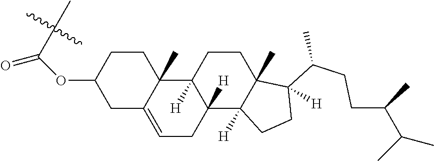 | Eribulin-campesterol conjugate with a cleavable carbamate bond |
| 60 | 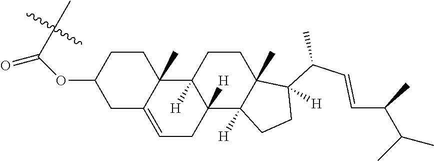 | Eribulin-brassicasterol conjugate with a cleavable carbamate bond |
| 61 | 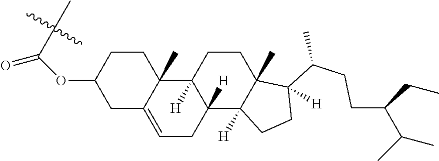 | Eribulin-sitosterol conjugate with a cleavable carbamate bond |
| 62 | 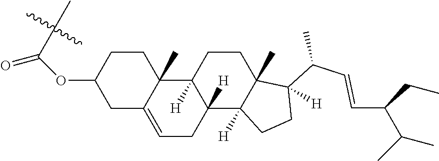 | Eribulin-stigmasterol conjugate with a cleavable carbamate bond |
| 63 | 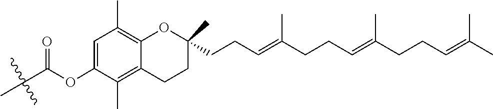 [SMILES: *C(=O)Oc1cc(C)c2c(c1C)CC[C@@](C)(CC/C=C(\C)CC/C=C(\C)CCC=C(C)C)O2] | Eribulin-β-tocotrienol conjugate with a cleavable carbamate bond |
| 64 | 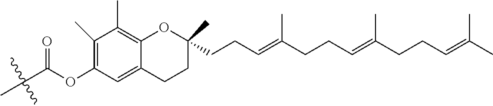 [SMILES: *C(=O)Oc1cc2c(c(C)c1C)O[C@](C)(CC/C=C(\C)CC/C=C(\C)CCC=C(C)C)CC2] | Eribulin-γ-tocotrienol conjugate with a cleavable carbamate bond |
| 65 | 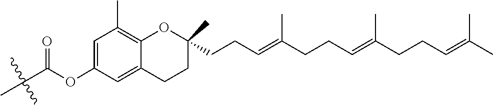 [SMILES: *C(=O)Oc1cc(C)c2c(c1)CC[C@@](C)(CC/C=C(\C)CC/C=C(\C)CCC=C(C)C)O2] | Eribulin-δ-tocotrienol conjugate with a cleavable carbamate bond |
| 66 | 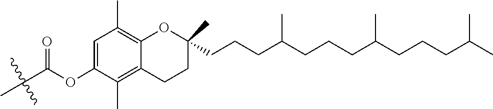 [SMILES: *C(=O)Oc1cc(C)c2c(c1C)CC[C@@](C)(CCCC(C)CCCC(C)CCCC(C)C)O2] | Eribulin-β-tocopherol conjugate with a cleavable carbamate bond |
| 67 | 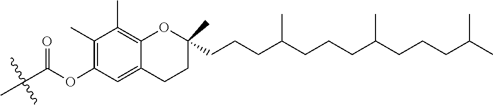 [SMILES: *C(=O)Oc1cc2c(c(C)c1C)O[C@](C)(CCCC(C)CCCC(C)CCCC(C)C)CC2] | Eribulin-γ-tocopherol conjugate with a cleavable carbamate bond |
| 68 | 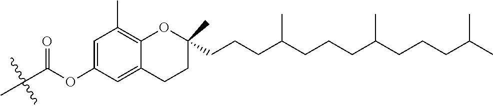 [SMILES: *C(=O)Oc1cc(C)c2c(c1)CC[C@@](C)(CCCC(C)CCCC(C)CCCC(C)C)O2] | Eribulin-δ-tocopherol conjugate with a cleavable carbamate bond |
| 69 | 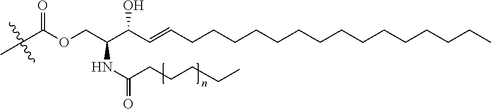 | n = 0 to 10 Eribulin-ceramide conjugate with a cleavable carbamate bond |
| 70 | 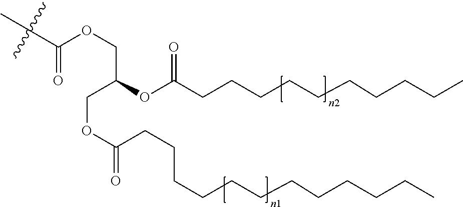 | n1 = 0 to 4 n2 = 0 to 4 Eribulin-diacylglycerol conjugate with a cleavable carbamate bond |

TABLE 6

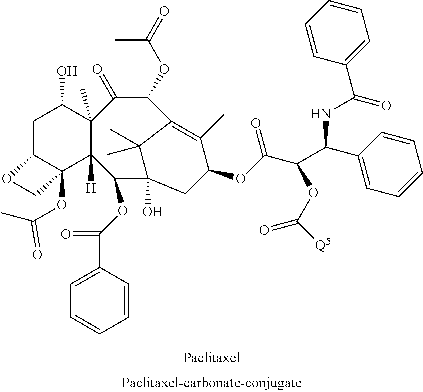

| No. | Q⁵ | Trivial Name |
|---|---|---|
| 71 | 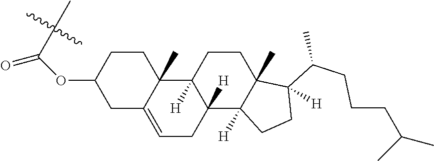 | Paclitaxel-cholesterol conjugate with a cleavable carbonate bond |
| 72 | 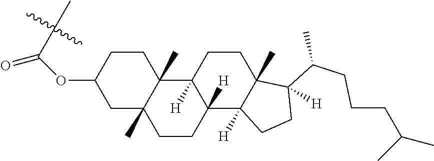 | Paclitaxel-coprostanol conjugate with a cleavable carbonate bond |
| 73 | 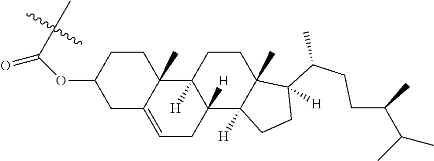 | Paclitaxel-campesterol conjugate with a cleavable carbonate bond |
| 74 | 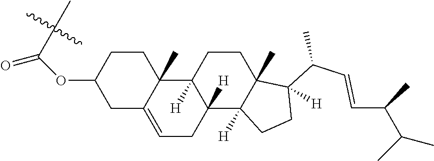 | Paclitaxel-brassicasterol conjugate with a cleavable carbonate bond |
| 75 | 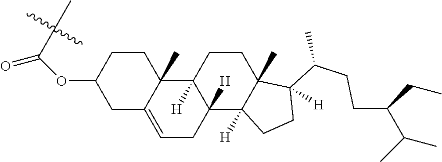 | Paclitaxel-sitosterol conjugate with a cleavable carbonate bond |
| 76 | 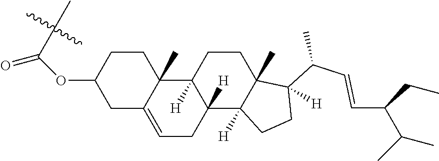 | Paclitaxel-stigmasterol conjugate with a cleavable carbonate bond |
| 77 | 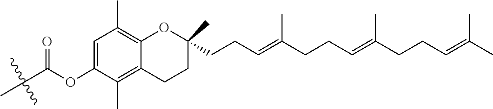 [SMILES: *C(=O)Oc1cc(C)c2c(c1C)CC[C@@](C)(CC/C=C(\C)CC/C=C(\C)CCC=C(C)C)O2] | Paclitaxel-β-tocotrienol conjugate with a cleavable carbonate bond |
| 78 | 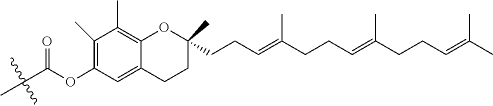 [SMILES: *C(=O)Oc1cc2c(c(C)c1C)O[C@](C)(CC/C=C(\C)CC/C=C(\C)CCC=C(C)C)CC2] | Paclitaxel-γ-tocotrienol conjugate with a cleavable carbonate bond |
| 79 | 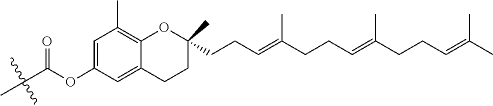 [SMILES: *C(=O)Oc1cc(C)c2c(c1)CC[C@@](C)(CC/C=C(\C)CC/C=C(\C)CCC=C(C)C)O2] | Paclitaxel-δ-tocotrienol conjugate with a cleavable carbonate bond |
| 80 | 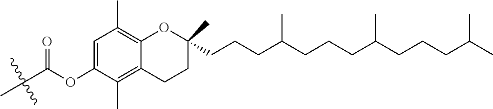 [SMILES: *C(=O)Oc1cc(C)c2c(c1C)CC[C@@](C)(CCCC(C)CCCC(C)CCCC(C)C)O2] | Paclitaxel-β-tocopherol conjugate with a cleavable carbonate bond |
| 81 | 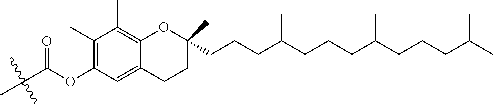 [SMILES: *C(=O)Oc1cc2c(c(C)c1C)O[C@](C)(CCCC(C)CCCC(C)CCCC(C)C)CC2] | Paclitaxel-γ-tocopherol conjugate with a cleavable carbonate bond |
| 82 | 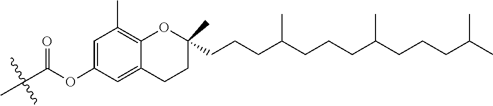 [SMILES: *C(=O)Oc1cc(C)c2c(c1)CC[C@@](C)(CCCC(C)CCCC(C)CCCC(C)C)O2] | Paclitaxel-δ-tocopherol conjugate with a cleavable carbonate bond |
| 83 | 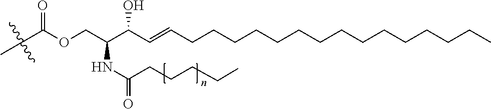 | n = 0 to 10 Paclitaxel-ceramide conjugate with a cleavable carbonate bond |
| 84 | 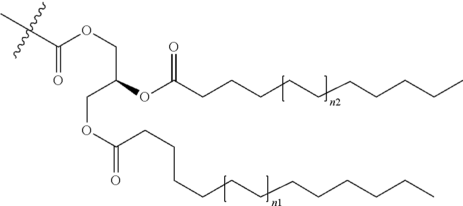 | n1 = 0 to 4 n2 = 0 to 4 Paclitaxel-diacylglycerol conjugate with a cleavable carbonate bond |

wherein the C-m-HDLs in the population have a hydrophilic exterior surface and a hydrophobic core; and
further wherein the C-m-HDLs in the population have an average particle diameter of from about 11.5 to about 14.0 nanometers, as measured by dynamic light scattering.

18. The population of claim 17, wherein said amphiphilic, alpha-helical peptide has an amino acid sequence as provided by SEQ ID NO: 25.

19. The population of claim 17, wherein said amphiphilic, alpha-helical peptide has an amino acid sequence as provided by SEQ ID NO: 28.

20. The population of claim 17, wherein said amphiphilic, alpha-helical peptide has an amino acid sequence as provided by SEQ ID NO: 34.

21. The population of claim 17, wherein said amphiphilic, alpha-helical peptide has an amino acid sequence as provided by SEQ ID NO: 35.

22. The population of claim 17, wherein said amphiphilic, alpha-helical peptide has an amino acid sequence as provided by SEQ ID NO: 36.

23. A method for preparing a solution of a fully dissolved amphiphilic, alpha-helical peptide selected from an amino acid sequence as provided by SEQ ID NO: 25, SEQ ID NO: 28, SEQ ID NO: 34, SEQ ID NO: 35, SEQ ID NO:36 and combinations thereof in an aqueous-ethanol co-solvent, which method comprises:
a) combining in any order from about 50 to about 90 weight percent ethanol and from about 7.5 or about 10 weight percent to about 50 weight percent water;
b) adding from about 2 to about 10 weight percent of an acid based on the weight of a) above;
c) adding from about 1 to about 2.5 weight percent of an amphiphilic, alpha-helical peptide selected from an amino acid sequence as provided by SEQ ID NO:25, SEQ ID NO:28, SEQ ID NO:34, SEQ ID NO:35 and SEQ ID NO:36 based on the total weight of the solvent solution of b) above; and
d) stirring until a clear solution is obtained.

24. A method for preparing Composition 1 which method comprises:
a) by combining in any order:
i) ethanol and water to form a cosolvent having from about 50 to about 90 weight percent ethanol and from about 7.5 or about 10 weight percent to about 50 weight percent water;
ii) about 2 to 10 weight percent of an acid based on the weight of the cosolvent;
iii) about 1 to 2.5 weight percent of a peptide of SEQ ID NO:25, SEQ ID NO: 28, SEQ ID NO:34; SEQ ID NO:35 or SEQ ID NO:36 based on the weight of the cosolvent of ii);
wherein the total amount of i), ii) and iii) equals 100%, and stirring the composition until the solution is clear evidencing that the peptide is completely dissolved; and
b) combining the solution generated in a) with from about 3 to about 6 weight percent of a lipid composition based on the weight of the solution of a) wherein said lipid composition comprises:
iv) about 50 to about 70 weight percent of 1-palmitoyl-2-oleoyl phosphatidylcholine;
v) about 18 to about 30 weight percent of sphingomyelin, and
vi) about 12 to about 35 weight percent of a conjugate of an anchor group and a chemotherapeutic agent covalently attached to each other by a cleavable bond;
and further wherein the combined solution generated in a) and the solution of b) equals 100%; and
c) stirring and filtering to provide for a clear solution, wherein the clear solution is designated as “Composition 1.”

25. The method of claim 24, wherein the conjugate comprising an anchor group and a chemotherapeutic agent covalently attached to each other by a cleavable bond is selected from a conjugate set forth in Tables 1-5.

26. The method of claim 24, wherein the resulting ranges for each of the components of Composition 1 is as follows:

| Compo- | | |
|---|---|---|
| nent | 94% Range | 97% Range/3% lipids |
| Ethanol | about 47.0% to about 84.6% | about 48.5% to about 87.3% |
| Water | about 7.05% to about 47.0% | about 7.3% to about 48.5%* |
| Acid | about 1.88% to about 09.4% | about 1.94% to about 9.70% |
| Peptide | about 0.94% to about 2.35% | about 0.97% to about 2.43% |
| POPC + | about 3.00% to about 4.2% | about 1.50% to about 2.10% |
| other | | |
| Lipids | | |
| SM | about 1.08% to about 1.80% | about 0.54% to about 0.90% |
| Conjugate | about 0.72% to about 2.10% | about 0.36% to about 1.05% |
| Benzyl | about 0% to about 7.5%** | about 0% to about 5%** |
| alcohol | | |

27. A method for reducing the size of micellar nanoparticles which method comprises:
a) combining water with from about 2 to 6 weight percent of a disaccharide and mix until homogeneous, wherein the homogeneous mixture is “Composition 2”;
b) adding Composition 2 to a first loading chamber of a three-chamber mixing device which comprises a second loading chamber and a single reaction chamber;
c) adding Composition 1 of claim 25 into the second loading chamber; and
d) chaotically mixing the two compositions into the reaction chamber under a controlled flow rate ratio of from about 4:1 to about 10:1 of Composition 2 to Composition 1 at a temperature of from about 30° to about 70° C. while maintaining the flow from each chamber until mixing is complete while maintaining a temperature of from about 30° to about 70° C.;
thereby providing a nanoparticle suspension having an average diameter for the nanoparticles of about 11.5 nanometers to about 14 nanometers.

28. The method of claim 27, wherein the disaccharide is sucrose.

29. The method of claim 27, wherein the proportions of components used are set to provide a final composition target of about 5 mg/mL of paclitaxel equivalents in an aqueous solution.

30. The method of claim 28, wherein the method further comprises removing at least a portion of the ethanol and acetic acid by either dialysis or tangential flow filtration.

31. The method of claim 30, wherein the composition is sterile filtered after dialysis or tangential flow filtration.

32. A method for treating a patient with a disorder mediated at least in part by the overexpression of the SR-BI receptor which method comprises administering to said patient an effective amount of a composition comprising micellular nanoparticles of claim 1.

33. The method of claim 32, wherein said disorder is a solid mass tumor that overexpresses SR-BI.

34. The method of claim 33, wherein said solid mass tumor is selected from breast cancer (including triple negative breast cancer), bladder cancer, gastrointestinal cancers, head and neck cancers, neuroblastoma, non-small-cell lung cancer, ovarian cancer, pancreatic cancer, prostate cancer, stomach cancer, kidney cancer, and cervical cancer.

35. The method of claim 34, wherein said composition is a pharmaceutical composition comprising a pharmaceutically acceptable excipient and about 5 mg/mL of paclitaxel equivalents in about a 4% by weight sucrose solution.

36. A method for treating a patient with a disorder mediated at least in part by the overexpression of SR-BI which method comprises administering to said patient an effective amount of a composition comprising micellular nanoparticles of claim 3.

37. The method of claim 36, wherein said disorder is a solid mass tumor that overexpresses SR-BI.

38. The method of claim 37, wherein said solid mass tumor is selected from breast cancer (including triple negative breast cancer), bladder cancer, gastrointestinal cancers, head and neck cancers, neuroblastoma, non-small-cell lung cancer, ovarian cancer, pancreatic cancer, prostate cancer, stomach cancer, kidney cancer, and cervical cancer.

39. The method of claim 38, wherein said composition is a pharmaceutical composition comprising a pharmaceutically acceptable excipient and about 5 mg/ml of paclitaxel equivalents in about a 4% by weight sucrose solution containing about 0.9 weight percent sodium chloride.

40. A pharmaceutical composition comprising a pharmaceutically acceptable excipient and an effective amount of a composition comprising micellular nanoparticles of claim 1.

41. A pharmaceutical composition comprising a pharmaceutically acceptable excipient and an effective amount of a composition comprising micellular nanoparticles of claim 3.

42. A pharmaceutical composition comprising an aqueous composition suitable for intravenous injection which composition comprise sterile water, sucrose and an effective amount of a population of C-m-HDLs which itself comprises:
(a) an amphiphilic, alpha-helical peptide that comprises an amino acid sequence of SEQ ID NO: 25, SEQ ID NO:28, SEQ ID NO:34, SEQ ID NO:35, or SEQ ID NO:36,
(b) one or both of sphingomyelin and phosphatidyl choline, and optionally additional lipid(s) including phospholipid(s); and
(c) a conjugate comprising paclitaxel and an anchor moiety, wherein the anchor moiety is releasably attached to paclitaxel through a carbonate bond; wherein said C-m-HDLs in said population comprise: a hydrophilic exterior surface; a hydrophobic core comprising the conjugate; wherein said population of micellar nanoparticles has an average particle diameter of about 11.5 to about 14 nanometers, as measured by dynamic light scattering.

43. The pharmaceutical composition of claim 42 wherein the population of micellar nanoparticles has an average particle diameter of about 12.0 to about 13.5 nanometers, as measured by dynamic light scattering.

44. The pharmaceutical composition of claim 43, wherein at least about 65% of the nanoparticles are within plus/minus about 2 nanometers of the average diameter.

45. The pharmaceutical composition of claim 43, wherein at least about 75% of the nanoparticles are within plus/minus about 2 nanometers of the average diameter.

46. A population of micellar nanoparticles comprising:
(a) an amphiphilic, alpha-helical peptide that comprises an amino acid sequence of any one of SEQ ID NO:25, SEQ ID NO:28, SEQ ID NO:34, SEQ ID NO:35, SEQ ID NO. 36, or combinations thereof;
(b) sphingomyelin and optionally phosphatidyl choline and/or one or more additional lipids or phospholipids;
(c) a conjugate comprising an anchor moiety molecule and a chemotherapeutic drug that are releasably attached to each other through a releasable bond; and
(d) a disaccharide,
wherein the micellar nanoparticles in the population further comprise:
a hydrophilic exterior surface and a hydrophobic core; and
a mean number average particle diameter of from about 12 to about 13.5 nanometers, as measured by dynamic light scattering.

47. The population of micellular nanoparticles of claim 46, wherein at least about 70% of said micellular nanoparticles are within plus/minus about 3 nanometers of the mean number average particle diameter of about 12 to about 13.5 nanometers.


SEQUENCE LISTING (36 sequences)

SEQ ID NO: 1 - protein, 20 aa, synthetic construct
  MOD_RES 3: note = L
  MOD_RES 3: note = F
  MOD_RES 5: note = A
  MOD_RES 5: note = Aib
  MOD_RES 6: note = L
  MOD_RES 6: note = F
  MOD_RES 9: note = A
  MOD_RES 9: note = L
  MOD_RES 9: note = F
  MOD_RES 9: note = Aib
  MOD_RES 10: note = L
  MOD_RES 10: note = F
  MOD_RES 11: note = A
  MOD_RES 11: note = Aib
  MOD_RES 11: note = N
  MOD_RES 13: note = L
  MOD_RES 13: note = F
  MOD_RES 14: note = A
  MOD_RES 14: note = L
  MOD_RES 14: note = F
  MOD_RES 14: note = Aib
  MOD_RES 17: note = W
  MOD_RES 17: note = F
  MOD_RES 17: note = L
DVXQXXKEXXXQXXEKXKQV

SEQ ID NO: 2 - protein, 20 aa, synthetic construct
DVFQALKELFAQLLEKWKQV

SEQ ID NO: 3 - protein, 20 aa, synthetic construct
  MOD_RES 5: note = Aib
DVFQXLKELFNQLLEKWKQV

SEQ ID NO: 4 - protein, 20 aa, synthetic construct
  MOD_RES 5: note = Aib
DVFQXLKELLAQLLEKFKQV

SEQ ID NO: 5 - protein, 20 aa, synthetic construct
  MOD_RES 5: note = Aib
DVFQXLKELLNQLLEKFKQV

SEQ ID NO: 6 - protein, 20 aa, synthetic construct
  MOD_RES 5: note = Aib
  MOD_RES 14: note = Aib
DVFQXLKELLNQLXEKFKQV

SEQ ID NO: 7 - protein, 20 aa, synthetic construct
  MOD_RES 5: note = Aib
  MOD_RES 14: note = Aib
DVFQXLKELLNQLXEKWKQV

SEQ ID NO: 8 - protein, 20 aa, synthetic construct
DVFQALKELLAQLLEKFKQV

SEQ ID NO: 9 - protein, 20 aa, synthetic construct
DVFQALKELLNQLLEKFKQV

SEQ ID NO: 10 - protein, 20 aa, synthetic construct
  MOD_RES 5: note = Aib
DVFQXLKELFAQLLEKWKQV

SEQ ID NO: 11 - protein, 20 aa, synthetic construct
  MOD_RES 5: note = Aib
DVFQXLKELFNQLLEKWKQV

SEQ ID NO: 12 - protein, 20 aa, synthetic construct
  MOD_RES 5: note = Aib
DVFQXLKELFNQLLEKFKQV

SEQ ID NO: 13 - protein, 20 aa, synthetic construct
  MOD_RES 14: note = Aib
DVFQALKELFAQLXEKWKQV

SEQ ID NO: 14 - skipped

SEQ ID NO: 15 - protein, 20 aa, synthetic construct
  MOD_RES 14: note = Aib
DVFQALKELFNQLXEKFKQV

SEQ ID NO: 16 - protein, 20 aa, unidentified
DVFQAFKEAFAQLFEKWKQV

SEQ ID NO: 17 - protein, 20 aa, synthetic construct
  MOD_RES 9: note = Aib
DVFQAFKEXFAQLFEKWKQV

SEQ ID NO: 18 - protein, 20 aa, synthetic construct
  MOD_RES 5: note = Aib
  MOD_RES 9: note = Aib
DVFQXFKEXFAQLFEKWKQV

SEQ ID NO: 19 - protein, 20 aa, synthetic construct
  MOD_RES 11: note = Aib
DVFQAFKEAFXQLFEKWKQV

SEQ ID NO: 20 - protein, 20 aa, synthetic construct
  MOD_RES 9: note = Aib
  MOD_RES 11: note = Aib
DVFQAFKEXFXQLFEKWKQV

SEQ ID NO: 21 - protein, 20 aa, synthetic construct
  MOD_RES 5: note = Aib
  MOD_RES 9: note = Aib
  MOD_RES 11: note = Aib
DVFQXFKEXFXQLFEKWKQV

SEQ ID NO: 22 - protein, 20 aa, synthetic construct
DVFQALKELFNQLLEKWKQV

SEQ ID NO: 23 - protein, 20 aa, synthetic construct
  MOD_RES 5: note = Aib
DVFQXLKELLNQLLEKLKQV

SEQ ID NO: 24 - skipped

SEQ ID NO: 25 - protein, 20 aa, synthetic construct
  MOD_RES 7: note = Aib
DVFQKLXELFNQLLEKWKQV

SEQ ID NO: 26 - protein, 20 aa, synthetic construct
DVFQKLVELFNQLLEKWKQV

SEQ ID NO: 27 - protein, 20 aa, synthetic construct
  MOD_RES 3: note = Aib
DVXQKLFELFNQLLEKWKQV

SEQ ID NO: 28 - protein, 20 aa, synthetic construct
  MOD_RES 7: note = Aib
DVFQKLXELFNQLLEKFKQV

SEQ ID NO: 29 - protein, 20 aa, synthetic construct
DVFQKLVELFNQLLEKFKQV

SEQ ID NO: 30 - protein, 20 aa, synthetic construct
  MOD_RES 3: note = Aib
DVXQKLFELFNQLLEKFKQV

SEQ ID NO: 31 - protein, 20 aa, synthetic construct
  MOD_RES 7: note = Aib
DVLQKFXELFNQLLEKWKQV

SEQ ID NO: 32 - protein, 20 aa, synthetic construct
  MOD_RES 3: note = Aib
DVXQKFLELFNQLLEKWKQV

SEQ ID NO: 33 - protein, 20 aa, synthetic construct
  MOD_RES 9: note = Aib
DVFQKLLEXFNQLLEKWKQV

SEQ ID NO: 34 - protein, 20 aa, synthetic construct
  MOD_RES 7: note = Aib
  MOD_RES 13: note = Aib
DVFQKLXELFNQXLEKWKQV

SEQ ID NO: 35 - protein, 20 aa, synthetic construct
  MOD_RES 7: note = Aib
  MOD_RES 14: note = Aib
DVFQKLXELFNQLXEKWKQV

SEQ ID NO: 36 - protein, 20 aa, synthetic construct
  MOD_RES 2: note = Aib
  MOD_RES 7: note = Aib
  MOD_RES 14: note = Aib
DXFQKLXELFNQLXEKWKQV